(19) United States
(12) Patent Application Publication
(10) Pub. No.: US 2012/0058999 A1
(43) Pub. Date: Mar. 8, 2012

(54) Substituted Tetrahydropyrrolopyrazine Compounds

(75) Inventors: Melanie REICH, Aachen (DE); Stefan SCHUNK, Aachen (DE); Stefan OBERBOERSCH, Aachen (DE); Ruth JOSTOCK, Stolberg (DE); Tieno GERMANN, Aachen (DE); Michael ENGELS, Turnhout (DE)

(73) Assignee: Gruenenthal GmbH, Aachen (DE)

(21) Appl. No.: 13/224,953

(22) Filed: Sep. 2, 2011

Related U.S. Application Data
(60) Provisional application No. 61/380,023, filed on Sep. 3, 2010.

(30) Foreign Application Priority Data
Sep. 3, 2010 (EP) 10009160.2

(51) Int. Cl. A61K 31/4985; A61K 31/5377; A61P 25/00; A61P 25/06; A61P 7/00; A61P 11/00; A61P 1/00; A61P 29/00; A61P 9/10; A61P 3/04; C07D 487/04; A61P 3/10

(52) U.S. Cl. 514/233.2; 544/349; 514/249; 544/119

(57) ABSTRACT

Tetrahydropyrrolopyrazine compounds, methods for their preparation, pharmaceutical compositions containing such compounds, and a method of using such compounds for the treatment of pain and other conditions mediated at least partially by Bradykinin 1 receptors (B1R).

### BACKGROUND OF THE INVENTION

The present invention relates to substituted tetrahydropyrrolopyrazine derivatives, processes for the preparation thereof, medicaments containing these compounds and the use of substituted tetrahydropyrrolopyrazine compounds for the preparation of medicaments.

In contrast to the constitutive expression of the bradykinin 2 receptor (B2R), in most tissues the bradykinin 1 receptor (B1R) is not expressed or is expressed only weakly. Nevertheless, expression of B1R can be induced on various cells. For example, in the course of inflammation reactions a rapid and pronounced induction of B1R takes place on neuronal cells, but also various peripheral cells, such as fibroblasts, endothelial cells, granulocytes, macrophages and lymphocytes. In the course of inflammation reactions, a switch from a B2R to a B1R dominance thus occurs on the cells involved. The cytokines interleukin-1 (IL-1) and tumour necrosis factor alpha (TNFα) are involved to a considerable degree in this upwards regulation of B1R (Passos et al. J. Immunol. 2004, 172, 1839-1847). After activation with specific ligands, B1R-expressing cells then themselves can secrete inflammation-promoting cytokines such as IL-6 and IL-8 (Hayashi et al., Eur. Respir. J. 2000, 16, 452-458). This leads to inwards migration of further inflammation cells, e.g. neutrophilic granulocytes (Pesquero et al., PNAS 2000, 97, 8140-8145). The bradykinin B1R system can contribute towards chronification of diseases via these mechanisms. This is demonstrated by a large number of animal studies (overviews in Leeb-Lundberg et al., Pharmacol Rev. 2005, 57, 27-77 and Pesquero et al., Biol. Chem. 2006, 387, 119-126). On humans too, an enhanced expression of B1R, e.g. on enterocytes and macrophages in the affected tissue of patients with inflammatory intestinal diseases (Stadnicki et al., Am. J. Physiol. Gastrointest. Liver Physiol. 2005, 289, G361-366) or on T lymphocytes of patients with multiple sclerosis (Pratet al., Neurology. 1999; 53,2087-2092) or an activation of the bradykinin B2R-B1R system in the course of infections with Staphyloccocus aureus (Bengtson et al., Blood 2006, 108, 2055-2063) is found. Infections with Staphyloccocus aureus are responsible for syndromes such as superficial infections of the skin up to septic shock.

Based on the pathophysiological relationships described, there is a great therapeutic potential for the use of B1R antagonists on acute and, in particular, chronically inflammatory diseases. These include diseases of the respiratory tract (bronchial asthma, allergies, COPD/chronic obstructive pulmonary disease, cystic fibrosis etc.), inflammatory intestinal diseases (ulcerative colitis, CD/Crohn's disease etc.), neurological diseases (multiple sclerosis, neurodegeneration etc.), inflammations of the skin (atopic dermatitis, psoriasis, bacterial infections etc.) and mucous membranes (Behcet's disease, pelvitis, prostatitis etc.), rheumatic diseases (rheumatoid arthritis, osteoarthritis etc.), septic shock and reperfusion syndrome (following cardiac infarction, stroke).

The bradykinin (receptor) system is moreover also involved in regulation of angiogenesis (potential as an angiogenesis inhibitor in cancer cases and macular degeneration on the eye), and B1R knockout mice are protected from induction of obesity by a particularly fat-rich diet (Pesquero et al., Biol. Chem. 2006, 387, 119-126). B1R antagonists are therefore also suitable for treatment of obesity.

B1R antagonists are suitable in particular for treatment of pain, in particular inflammation pain and neuropathic pain (Calixto et al., Br. J. Pharmacol 2004, 1-16), and here in particular diabetic neuropathy (Gabra et al., Biol. Chem. 2006, 387, 127-143). They are furthermore suitable for treatment of migraine.

In the development of B1R modulators, however, there is the problem that the human and the rat B1 receptor differ so widely that many compounds which are good B1R modulators on the human receptor have only a poor or no affinity for the rat receptor. This makes pharmacological studies on animals considerably difficult, since many studies are usually conducted on the rat. However, if no activity exists on the rat receptor, neither the action nor side effects can be investigated on the rat. This has already led to transgenic animals with human B1 receptors being produced for pharmacological studies on animals (Hess et al., Biol. Chem. 2006; 387(2):195-201). Working with transgenic animals, however, is more expensive than working with the unmodified animals.

PCT patent application nos. WO 2008/040492 and WO 2008/046573 describe compounds which, in in vitro assays, show an antagonistic action both on the human B1 receptor and on the B1 receptor of the rat.

PCT patent application nos. WO 2007/140383 and WO 2007/101007 describe compounds which have an antagonistic action on the macaque B1 receptor in in vitro assays. Experimental data on the activity on the human B1 receptor or the B1 receptor of the rat are not disclosed.

PCT patent application nos. WO 2009/021944 and WO2010/017850 describe cycloalkly-substituted piperazine compounds which exhibit antagonistic action on the human B1R receptor.

There continues to be a need for novel B1R modulators, B1R modulators which bind both to the rat receptor and to the human receptor offering particular advantages.

### SUMMARY OF THE INVENTION

One object of the present invention was therefore to provide novel compounds which are suitable in particular as pharmacological active compounds in medicaments, preferably in medicaments for treatment of disorders or diseases which are at least partly mediated by B1R receptors.

This object is achieved by the substituted tetrahydropyrrolopyrazine derivatives according to the invention.

The present invention therefore provides substituted tetrahydropyrrolopyrazine derivatives compounds of the general formula (I)

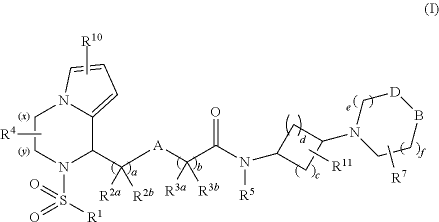

in the form of a single enantiomer or a single diastereomer, the racemate, the enantiomers, the diastereomers, mixtures of the enantiomers and/or diastereomers, and each in the form of their bases and/or physiologically acceptable salts, wherein
a represents 0, 1 or 2;
b represents 1 or 2;
A represents C(R^{6a})(R^{6b}), O or a single bond;
R¹ represents aryl or heteroaryl;
R^{2a}, R^{2b}, R^{3a}, R^{3b}, R^{6a} and R^{6b} are mutually independently selected from H, OH and O—C₁₋₆ alkyl;
R⁴ represents 0 to 4 substituents which are mutually independently selected from C₁₋₆ alkyl and C₃₋₈ cycloalkyl; or
R⁴ represents an anellated aryl or heteroaryl bonded to the carbon atoms denoted in the general formula (I) by the letters (x) and (y);
R⁵ represents H; C₁₋₆ alkyl; C₃₋₈ cycloalkyl or C₃₋₈ cycloalkyl bonded by a C₁₋₆ alkylene group;
R⁷ represents 0 to 4 substituents which are mutually independently selected from F; Cl; OH; ═O; C₁₋₆ alkyl; O—C₁₋₆ alkyl; C₃₋₈ cycloalkyl; aryl or heteroaryl; C₃₋₈ cycloalkyl, aryl or heteroaryl bonded by a C₁₋₈ alkylene group;
c represents 1, 2 or 3,
d represents 1, 2 or 3,
with the provisio that c+d is equal or less than 4,
e and f are each independently selected from 0 or 1,
with the provisio that e+f is not 0;
B represents NR⁸, O, CH—N(R^{9a})(R^{9b}), or CF₂,
wherein
R⁸ represents H, C₁₋₆ alkyl or C₃₋₈ cycloalkyl,
R^{9a} and R^{9b} are each independently selected from H, C₁₋₄ alkyl and C₃₋₈ cycloalkyl or
R^{9a} and R^{9b} together with the N-atom to which they are bonded to form a 4-, 5- or 6-membered heterocyclyl group,
R¹⁰ represents 0 to 2 substituents which are mutually independently selected from CH₃, CF₃, F and Cl,
R¹¹ represents 0 to 4 substituents which are mutually independently selected from F; Cl; OH; ═O; C₁₋₆ alkyl; O—C₁₋₆ alkyl; C₃₋₈ cycloalkyl; aryl or heteroaryl; C₃₋₈ cycloalkyl, aryl or heteroaryl bonded by a C₁₋₆ alkylene group;
D represents CH₂; or
D together with B forms an anellated, 5- or 6-membered heteroaryl or aryl;
wherein the aforementioned radicals C₁₋₃, C₁₋₄ and C₁₋₆ alkyl, C₁₋₃ and C₁₋₆ alkylene, C₃₋₆-cycloalkyl, C₃₋₈-cycloalkyl, aryl, heteroaryl and heterocyclyl can each be unsubstituted or mono- or polysubstituted with identical or different radicals; the aforementioned radicals C₁₋₃, C₁₋₄ and C₁₋₆ alkyl, C₁₋₃ and C₁₋₆ alkylene can each be branched or unbranched.

Within the scope of the present invention the term “halogen” preferably stands for the radicals F, Cl, Br and I, in particular for the radicals F and Cl.

Within the scope of this invention, the expression “C₁₋₆ alkyl” includes acyclic saturated hydrocarbon radicals having 1, 2, 3, 4, 5 or 6 C atoms, which can be branched or straight-chain (unbranched) and unsubstituted or mono- or polysubstituted, for example di-, tri-, tetra- or pentasubstituted, with identical or different radicals. The alkyl radicals can preferably be selected from the group consisting of methyl, ethyl, n-propyl, isopropyl, n-butyl, isobutyl, sec-butyl, tert-butyl, n-pentyl, isopentyl, neopentyl and hexyl. Particularly preferred alkyl radicals can be selected from the group consisting of methyl, ethyl, n-propyl, isopropyl, n-butyl, sec-butyl, isobutyl and tert-butyl.

From the above It is clear that any corresponding expressions which differ only via the numerical range indicated by the index numbers, e.g. “C₃₋₆ cycloalkyl” or “C₃₋₄ cycloalkyl” have a corresponding meaning defined by said numerical ranges. This applies in a corresponding manner to all the definitions outlined herein.

Within the meaning of this invention, the expression “C₃₋₈ cycloalkyl” denotes cyclic saturated hydrocarbons having 3, 4, 5, 6, 7 or 8 carbon atoms, which can be unsubstituted or mono- or polysubstituted, for example di-, tri-, tetra- or pentasubstituted, at one or more ring members with identical or different radicals. C₃₋₈ cycloalkyl can preferably be selected from the group consisting of cyclopropyl, cyclobutyl, cyclopentyl, cyclohexyl, cycloheptyl and cyclooctyl.

Within the scope of this invention, the expression “aryl” denotes aromatic hydrocarbons, in particular phenyls and naphthyls. The aryl radicals can also be fused to other saturated, (partially) unsaturated or aromatic ring systems. Each aryl radical can be present in unsubstituted or mono- or polysubstituted form, for example di-, tri-, tetra- or pentasubstituted, wherein the aryl substituents can be identical or different and can be at any desired and possible position of the aryl. Aryl can advantageously be selected from the group consisting of phenyl, 1-naphthyl and 2-naphthyl, which can be unsubstituted or mono- or polysubstituted, for example with 2, 3, 4 or 5 radicals.

Within the scope of the present invention, the expression “heteroaryl” stands for a 5-, 6- or 7-membered cyclic aromatic radical containing at least 1, optionally also 2, 3, 4 or 5 heteroatoms, wherein the heteroatoms can be identical or different and the heteroaryl can be unsubstituted or mono- or polysubstituted, for example di-, tri-, tetra- or pentasubstituted, with identical or different radicals. The substituents can be bound to any desired and possible position of the heteroaryl. The heterocyclic compound can also be part of a bicyclic or polycyclic, in particular a mono-, bi- or tricyclic system, which can then in total be more than 7-membered, preferably up to 14-membered. Preferred heteroatoms are selected from the group consisting of N, O and S. The heteroaryl radical can preferably be selected from the group consisting of pyrrolyl, indolyl, furyl (furanyl), benzofuranyl, thienyl (thiophenyl), benzothienyl, benzothiadiazolyl, benzothiazolyl, benzotriazolyl, benzodioxolanyl, benzodioxanyl, benzooxazolyl, benzooxadiazolyl, imidazothiazolyl, dibenzofuranyl, dibenzothienyl, phthalazinyl, pyrazolyl, imidazolyl, thiazolyl, oxazolyl, oxadiazolyl, triazole, tetrazole, isoxazoyl, pyridinyl (pyridyl), pyridazinyl, pyrimidinyl, pyrazinyl, pyranyl, indazolyl, purinyl, indolizinyl, quinolinyl, isoquinolinyl, quinazolinyl, quinoxalinyl, carbazolyl, phenazinyl, phenothiazinyl and oxadiazolyl, in particular from the group consisting of thienyl (thiophenyl), pyridinyl (pyridyl), pyrimidinyl, thiazolyl, triazolyl, imidazolyl, oxazolyl, oxadiazolyl, quinazolinyl, quinolinyl and isoquinolinyl, wherein the binding to the general structure (I) can be made via any desired and possible ring member of the heteroaryl radical. The heteroaryl radical can particularly preferably be selected from the group consisting of thienyl, imidazolyl, thiazolyl, triazolyl, pyridinyl and pyrimidinyl.

In the context of the present invention the expression “4-, 5- or 6-membered heterocyclyl”, unless specified further, includes both aromatic and saturated or partially unsaturated 4-, 5- or 6-membered cyclic hydrocarbon compounds in which one or more carbon ring members is mutually independently replaced by a heteroatom or a heteroatom group, in particular by N, O or S.

For example, 1, 2 or 3 ring atoms in the heterocyclyl can be heteroatoms. Non-aromatic heterocyclyls can be unsubstituted, mono- or polysubstituted with identical or different substituents, wherein the substituents correspond to those described below in connection with the substitution of C₃₋₈ cycloalkyls.

Aromatic heterocyclyls are synonymous with heteroaryls. The meaning of the term “heteroaryl” has already been described above and the possible substitution is likewise explained below.

Examples of 4- to 6-membered heterocyclyls include firstly the 5- or 6-membered heteroaryls already mentioned in connection with heteroaryls and secondly also pyrrolidinyl, piperidinyl, 2,6-dimethylpiperidine, 4,5-dihydro-1H-imidazo-2-yl or 1-methyl-4,5-dihydroimidazo-2-yl. In connection with the 4- to 7-membered heterocyclyl group in the radical R¹¹ this can be selected in particular from pyrrolidinyl, piperidinyl, 2,6-dimethylpiperidine, 1H-pyrrol-1-yl, 1H-pyrrol-2-yl, 4,5-dihydro-1H-imidazo-2-yl, 1-methyl-4,5-dihydroimidazo-2-yl or 4H-1,2,4-triazol-4-yl, each unsubstituted or optionally mono- or polysubstituted.

Examples of 4- to 6-membered heterocycloalkyls include azetidinyl, pyrrolidinyl, piperidinyl, piperazinyl, morpholinyl, tetrahydropyranyl, dioxanyl and dioxolanyl, which can optionally be substituted as described below.

Within the meaning of the present invention, the expression “C₁₋₃ alkylene group” or “C₁₋₆ alkylene group” includes acyclic saturated hydrocarbon radicals having respectively 1, 2 or 3 or 1, 2, 3, 4, 5 or 6 C atoms, which can be branched or straight-chain (unbranched) and unsubstituted or mono- or polysubstituted, for example di-, tri-, tetra- or pentasubstituted, with identical or different radicals and which link a corresponding radical to the higher-order general structure. The alkylene groups can preferably be selected from the group consisting of —CH₂—, —CH₂—CH₂—, —CH(CH₃)—, —CH₂—CH₂—CH₂—, —CH(CH₃)—CH₂—, —CH(CH₂CH₃)—, —CH₂—(CH₂)₂—CH₂—, —CH(CH₃)—CH₂—CH₂—, —CH₂—CH(CH₃)—CH₂—, —CH(CH₃)—CH(CH₃)—, —CH(CH₂CH₃)—CH₂—, —C(CH₃)₂—CH₂—, —CH(CH₂CH₂CH₃)—, —C(CH₃)(CH₂CH₃)—, —CH₂—(CH₂)₃—CH₂—, —CH(CH₃)—CH₂—CH₂—CH₂—, —CH₂—CH(CH₃)—CH₂—CH₂—, —CH(CH₃)—CH₂—CH(CH₃)—, —CH(CH₃)—CH(CH₃)—CH₂—, —C(CH₃)₂—CH₂—CH₂—, —CH₂—C(CH₃)₂—CH₂—, —CH(CH₂CH₃)—CH₂—CH₂—, —CH₂—CH(CH₂CH₃)—CH₂—, —C(CH₃)₂—CH(CH₃)—, —CH(CH₂CH₃)—CH(CH₃)—, —C(CH₃)(CH₂CH₃)—CH₂—, —CH(CH₂CH₂CH₃)—CH₂—, —C(CH₂CH₂CH₃)—CH₂—, —CH(CH₂CH₂CH₂CH₃)—, —C(CH₃)(CH₂CH₂CH₃)—, —C(CH₂CH₃)₂— and —CH₂—(CH₂)₄—CH₂—. The alkylene groups can particularly preferably be selected from the group consisting of —CH₂—, —CH₂—CH₂— and —CH₂—CH₂—CH₂—.

Within the meaning of the present invention, the expression “C₂₋₆ alkenylene group” includes acyclic mono- or polyunsaturated, for example di-, tri- or tetraunsaturated, hydrocarbon radicals having 2, 3, 4, 5 or 6 C atoms, which can be branched or straight-chain (unbranched) and unsubstituted or mono- or polysubstituted, for example di-, tri-, tetra- or pentasubstituted, with identical or different radicals and which link a corresponding radical to the higher-order general structure. The alkenylene groups include at least one C═C double bond. The alkenylene groups can preferably be selected from the group consisting of —CH═CH—, —CH═CH—CH₂—, —C(CH₃)═CH₂—, —CH═CH—CH₂—CH₂—, —CH₂—CH═CH—CH₂—, —CH═CH—CH═CH—, —C(CH₃)═CH—CH₂—, —CH═C(CH₃)—CH₂—, —C(CH₃)═C(CH₃)—, —C(CH₂CH₃)═CH—, —CH═CH—CH₂—CH₂—CH₂—, —CH₂—CH═CH₂—CH₂—CH₂—, —CH═CH═CH—CH₂—CH₂— and —CH═CH₂—CH—CH═CH₂—.

Within the meaning of the invention, the expression “C₂₋₆ alkynylene group” includes acyclic mono- or polyunsaturated, for example di-, tri- or tetraunsaturated, hydrocarbon radicals having 2, 3, 4, 5 or 6 C atoms, which can be branched or straight-chain (unbranched) and unsubstituted or mono- or polysubstituted, for example di-, tri-, tetra- or pentasubstituted, with identical or different radicals and which link a corresponding radical to the higher-order general structure. The alkynylene groups include at least one C≡C triple bond. The alkynylene groups can preferably be selected from the group consisting of —C≡C—, —C≡C—CH₂—, —C≡C—CH₂—CH₂—, —C≡C—CH(CH₃)—, —CH₂—C≡C—CH₂—, —C≡C—C≡C—, —C≡C—C(CH₃)₂—, —C≡C—CH₂—CH₂—CH₂—, —CH₂—C≡C—CH₂—CH₂—, —C≡C—C≡C—CH₂— and —C≡C—CH₂—C≡C—.

Within the meaning of the present invention the expression “aryl or heteroaryl bound by a C₁₋₃ alkylene group, a C₁₋₆ alkylene group, C₂₋₆ alkenylene group or C₂₋₆ alkynylene group” means that the C₁₋₃ alkylene groups, C₁₋₆ alkylene groups, C₂₋₆ alkenylene groups, C₂₋₆ alkynylene groups and aryl or heteroaryl have the meanings defined above and the aryl or heteroaryl is bound to the higher-order general structure by a C₁₋₃ alkylene group, C₁₋₆ alkylene group, C₂₋₆ alkenylene group or C₂₋₆ alkynylene group. Benzyl, phenethyl and phenylpropyl are cited by way of example.

Within the meaning of the present invention, the expression “C₃₋₈ cycloalkyl, 3- to 6-membered or 4- to 7-membered heterocyclyl or 3- to 8-membered heterocycloalkyl bound by a C₁₋₃ alkylene group, C₁₋₆ alkylene group, C₂₋₆ alkenylene group or C₂₋₆ alkynylene group” means that the C₁₋₃ alkylene group, C₁₋₆ alkylene group, C₂₋₆ alkenylene group, C₂₋₆ alkynylene group, C₃₋₈ cycloalkyl and heterocycloalkyl have the meanings defined above and C₃₋₈ cycloalkyl and heterocycloalkyl are bound to the higher-order general structure by a C₁₋₃ alkylene group, C₁₋₆ alkylene group, C₂₋₆ alkenylene group or C₂₋₆ alkynylene group.

As used herein the term “isolated” with respect to a diastereomer or enantiomer means substantially separated from other stereoismers but not necessarily from other substances.

In connection with “alkyl”, “alkylene”, “alkenylene”, “alkynylene” and “cycloalkyl”, the term “substituted” within the meaning of this invention is understood to mean the substitution of a hydrogen radical with F, Cl, Br, I, CF₃, OCF₃, CN, NH₂, NH—C₁₋₆ alkyl, NH—C₁₋₆ alkylene-OH, C₁₋₆ alkyl, N(C₁₋₆ alkyl)₂, N(C₁₋₆ alkylene-OH)₂, NO₂, SH, S—C₁₋₆ alkyl, C₁₋₆ alkyl, S-benzyl, O—C₁₋₆ alkyl, OH, O—C₁₋₆ alkylene-OH, ═O, O-benzyl, C(═O)C₁₋₆ alkyl, CO₂H, CO₂—C₁₋₆ alkyl, phenyl, phenoxy, benzyl, naphthyl, furyl, thienyl and pyridinyl, wherein polysubstituted radicals are understood to mean radicals which are substituted multiple times, for example twice or three times, at different or the same atoms, for example substituted three times at the same C atom, as in the case of CF₃ or CH₂CF₃, or at different sites, as in the case of CH(Cl)—CH═CH—CHCl₂. The polysubstitution can take place with identical or different substituents, as for example in the case of CH(OH)—CH═CH—CHCl₂. It should be understood in particular to be the substitution of one or more hydrogen radicals with F, Cl, NH₂, OH, phenyl, O—CF₃ or O—C₁₋₆ alkyl, in particular methoxy.

In connection with “aryl” and “heteroaryl”, the term “substituted” within the meaning of this invention is understood to mean the mono- or polysubstitution, for example the di-, tri-, tetra- or pentasubstitution, of one or more hydrogen atoms of the corresponding ring system with F, Cl, Br, I, CN, NH₂, NH—C₁₋₆ alkyl, NH—C₁₋₆ alkylene-OH, N(C₁₋₆ alkyl)₂, N(C₁₋₆ alkylene-OH)₂, NH-aryl1, N(aryl1)₂, N(C₁₋₆ alkyl)aryl1, pyrrolinyl, piperazinyl, morpholinyl, azetidinyl, piperidinyl, thiazolinyl, azepanyl, diazepanyl, (C₁₋₃ alkylene)-azetidinyl, (C₁₋₃ alkylene)-pyrrolinyl, (C₁₋₃ alkylene)-piperidinyl, (C₁₋₃ alkylene)-morpholinyl, (C₁₋₃ alkylene)-piperazinyl, (C₁₋₃ alkylene)-thiazolinyl, (C₁₋₃ alkylene)-azepanyl, (C₁₋₃ alkylene)-diazepanyl, NO₂, SH, S—C₁₋₆ alkyl, OH, O—C₁₋₆ alkyl, O—C₁₋₆ alkyl-OH, C(═O)C₁₋₆ alkyl, NHSO₂C₁₋₆ alkyl, NHCOC₁₋₆ alkyl, CO₂H, CH₂SO₂ phenyl, CO₂—C₁₋₆ alkyl, OCF₃, CF₃, —O—CH₂—O—, —O—CH₂—CH₂—O—, —O—C(CH₃)₂—CH₂—, unsubstituted C₁₋₆ alkyl, pyrrolidinyl, imidazolyl, benzyloxy, phenoxy, phenyl, naphthyl, pyridinyl, —C₁₋₃ alkylene-aryl1, benzyl, thienyl, furyl, wherein aryl1 stands for phenyl, thiazolyl, thienyl or pyridinyl, at one or different atoms, wherein the aforementioned substituents—unless otherwise specified—can themselves be substituted with the cited substituents. The polysubstitution of aryl and heteroaryl can be performed with identical or different substituents. Preferred substituents for aryl and heteroaryl can be selected from the group consisting of —O—C₁₋₃ alkyl, unsubstituted C₁₋₆ alkyl, F, Cl, Br, I, CN, CF₃, OCF₃, OH, SH, —CH₂ azetidinyl, —CH₂-pyrrolidinyl, —CH₂-piperidinyl, —CH₂-piperazinyl, —CH₂-morpholinyl, phenyl, naphthyl, thiazolyl, thienyl and pyridinyl, in particular from the group consisting of F, Cl, CN, CF₃, CH₃; OCH₃, OCF₃ and —CH₂-azetidinyl.

In connection with “anellated aryl” or “anellated heteroaryl”, “substituted” within the meaning of this invention is understood to mean, in addition to the possible substituents and substitution models defined above in connection with “aryl” and “heteroaryl”, 4- to 7-membered heterocyclyls as further possible substituents, which have the meaning defined above and can optionally be connected to the anellated aryl or heteroaryl by a C₁₋₃ cycloalkylene group. In particular the 4- to 7-membered heterocyclyls appearing as substituents can be selected from the group consisting of morpholinyl, azetidinyl, piperidinyl, thiazolinyl, azepanyl, diazepanyl, (C₁₋₃ alkylene)-azetidinyl, (C₁₋₃ alkylene)-pyrrolinyl, (C₁₋₃ alkylene)-piperidinyl, (C₁₋₃ alkylene)-morpholinyl, (C₁₋₃ alkylene)-piperazinyl, (C₁₋₃ alkylene)-thiazolinyl, (C₁₋₃ alkylene)-azepanyl, (C₁₋₃ alkylene)-diazepanyl, pyrrolidinyl, 2,6-dimethylpiperidine, (C₁₋₃ alkylene)-2,6-dimethylpiperidine, 1H-pyrrol-1-yl, (C₁₋₃ alkylene)-1H-pyrrol-1-yl, 1H-pyrrol-2-yl, (C₁₋₃ alkylene)-1H-pyrrol-2-yl, 4,5-dihydro-1H-imidazo-2-yl, (C₁₋₃ alkylene)-4,5-dihydro-1H-imidazo-2-yl, 1-methyl-4,5-dihydroimidazo-2-yl, (C₁₋₃ alkylene)-1-methyl-4,5-dihydroimidazo-2-yl or 4H-1,2,4-triazol-4-yl-(C₁₋₃ alkylene)-4H-1,2,4-triazol-4-yl, each unsubstituted or optionally mono- or polysubstituted, as defined above. In particular, the 4- to 7-membered heterocyclyls may be substituted with two adjacent substituents which together form an anellated aryl or heteroaryl, especially an anellated phenyl. Especially, piperidinyl may be substituted in such a manner that an 1,2,3,4-tetrahydroquinolinyl or 1,2,3,4-tetrahydroisoquinolinyl is formed.

In connection with non-aromatic heterocyclyls the term “substituted” is understood to mean the substitution of a hydrogen radical at one or more ring members by F, Cl, Br, I, —CN, NH₂, NH—C₁₋₆ alkyl, NH—C₁₋₆ alkylene-OH, C₁₋₆ alkyl, N(C₁₋₆ alkyl)₂, N(C₁₋₆ alkylene-OH)₂, pyrrolinyl, piperazinyl, morpholinyl, NO₂, SH, S—C₁₋₆ alkyl, S-benzyl, O—C₁₋₆ alkyl, OH, O—C₁₋₆ alkylene-OH, ═O, O-benzyl, C(═O)C₁₋₆ alkyl, CO₂H, CO₂—C₁₋₆ alkyl or benzyl. The polysubstitution can be performed with identical or different substituents. A hydrogen bound to an N ring member can be substituted with a C₁₋₆ alkyl, C₃₋₈ cycloalkyl, aryl, heteroaryl or a C₃₋₈ cycloalkyl, aryl or heteroaryl bound by a C₁₋₃ alkylene group, wherein these alkyl, cycloalkyl, alkylene and aryl and heteroaryl groups can be unsubstituted or substituted as defined above. Examples of substituted 3- to 8-membered heterocycloalkyl groups include 1-methylpiperidin-4-yl, 1-phenylpiperidin-4-yl, 1-benzylpiperidin-4-yl, 1-methylpyrrolidin-3-yl, 1-phenylpyrrolidin-3-yl, 1-benzylpyrrolin-3-yl, 1-methylazetidin-3-yl, 1-phenyl-azetidin-3-yl or 1-benzylazetidin-3-yl.

In the chemical structural formulae which are used here to describe the compounds according to the invention, the symbol “

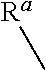

” is also used to describe one or more substitution models, wherein unlike the representation of a binding to a specific atom, this group is not bound to a specific atom within the chemical structural formula (R^{a} stands by way of example here for a substituent R identified by the number represented by the variable “a”).

In the context of the present invention, the symbol

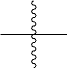

used in formulas represents a linking of a corresponding radical to the higher-order general structure.

The person skilled in the art understands that identical radicals used for the definition of different substituents are mutually independent.

Within the meaning of this invention the term “physiologically compatible salt” is understood to mean preferably salts of the compounds according to the invention with inorganic or organic acids, which are physiologically—particularly when used in humans and/or mammals—compatible; e.g. salts formed with hydrochoric acid (hydrochlorides) and with citric acid (citrates).

In certain embodiments of the invention, the compounds are those wherein in general formula (I) A represents O and each of R^{2a}, R^{2b}, R^{3a} and R^{3b} represents H; or A represents C(R^{6a})(R^{6b}) or a single bond and R^{2a}, R^{2b}, R^{3a}, R^{3b}, R^{6a} and R^{6b} mutually independently represent H, F, CF₃, OH, CH₃, O—CH₃ or O—CF₃, with the provisio that out of R^{2a}, R^{2b}, R^{3a}, R^{3b}, R^{6a} and R^{6b} only up to two of these groups can represent a group other than H at the same time. In specific embodiments of the compounds according to the present invention all of R^{2a}, R^{2b}, R^{3a}, R^{3b} and R^{6a} and R^{6b}, if present, represent H.

In further embodiments of the inventive compounds defined in general formula (I), a represents 0, A represents a single bond and b represents 1; a and b each represent 1 and A represents a single bond or CR^{6a}R^{6b}; or a and b each represent 1 and A represents O.

In embodiments of the inventive compounds defined in general formula (I), R¹ represents phenyl or naphthyl, each unsubstituted or mono- or polysubstituted, identically or differently, wherein the substituents are selected from —O—C₁₋₃ alkyl, C₁₋₆ alkyl, F, Cl, Br, CF₃ or OCF₃. In certain embodiments the substituents are selected from OCH₃, CH₃, F, C₁ and CF₃.

Compounds according to the present invention may be compounds as defined by general formula Ia below:

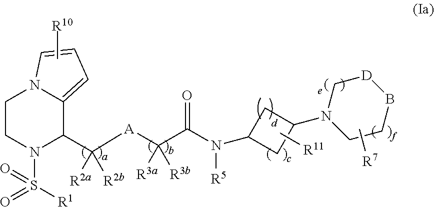

wherein all of the residues and variables may have the meanings as defined herein. In certain embodiments of the invention, the variables e and f in the compounds according to general formula I or Ia both represent 1.

In certain embodiments of the inventive compounds according to general formula I or Ia, R⁵ represents H; C₁₋₆ alkyl; C₃₋₈ cycloalkyl; aryl or C₃₋₈ cycloalkyl bonded via a C₁₋₃ alkylene group. In particular embodiments of the inventive compounds, R⁵ represents H, Me, Et, Pr, isopropyl, iso-butyl, tert butyl, CH₂CF₃, cyclopropyl or cyclobutyl.

In certain embodiments of the compounds according to the present invention as defined in general formulas I or Ia, c represents 1 and d represents 3 or c represents 3 and d represents 1. In other embodiments c and d both represent 1. In still other embodiments c and d both represent 2. In further variations within the compounds of the present invention, c represents 1 and d represents 2 or c represents 2 and d represents 1.

In certain compounds according to the present invention R¹¹ is selected from the group consisting of H, C₁₋₆ alkyl or C₃₋₈ cycloalkyl, and may especially represent H.

In certain embodiments of the compounds according to the present invention:
B represents NR⁸ wherein R⁸ is selected from H, C₁₋₄ alkyl or C₃₋₆ cycloalkyl and D represents CH₂;
B represents O and D represents CH₂;
B represents CH—N(R^{9a})(R^{9b}), wherein R^{9a} and R^{9b} are each independently selected from H, C₁₋₃ alkyl and C₃₋₄ cycloalkyl or R^{9a} and R^{9b} together with the N-atom to which they are bonded to form a 4- or 5-membered heterocycle and D represents CH₂; or
B and D together form an anellated pyridinyl moiety.

In certain embodiments of the compounds according to the present invention the substructure within general formula I represented by general formula II

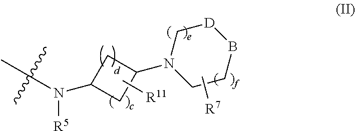

is selected from

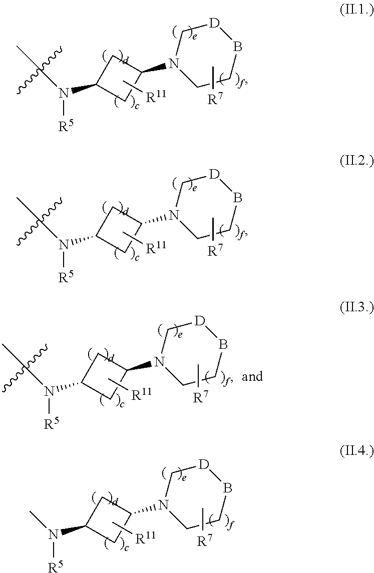

wherein
R⁵, R⁷, R¹¹, c, d, e, f, B and D may have any of the meanings as defined herein. In certain embodiments of the inventive compounds e and f both represent 1.

In specific embodiments of the inventive compounds the substructure within general formula I represented by general formula II.a

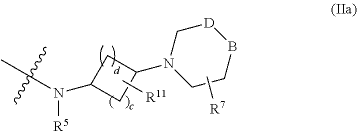

is selected from

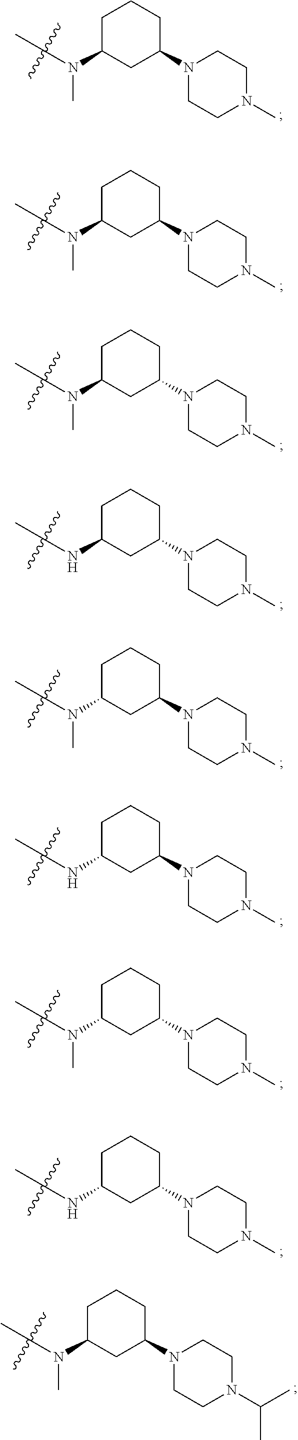

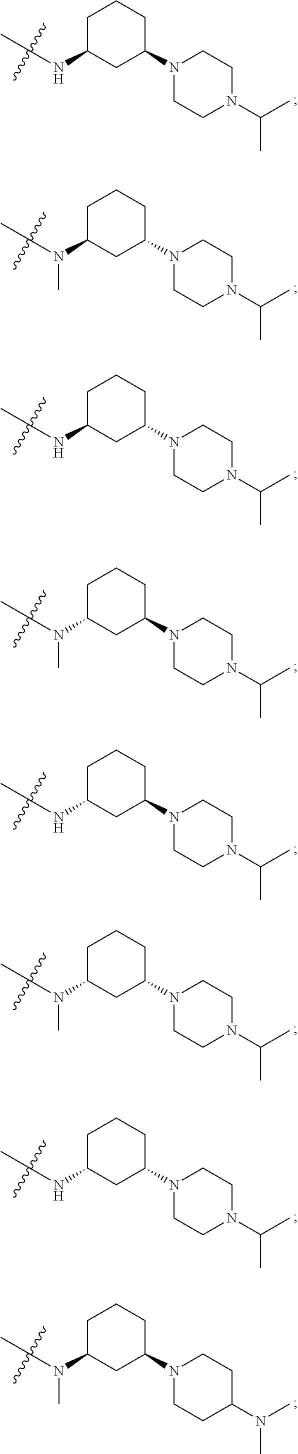

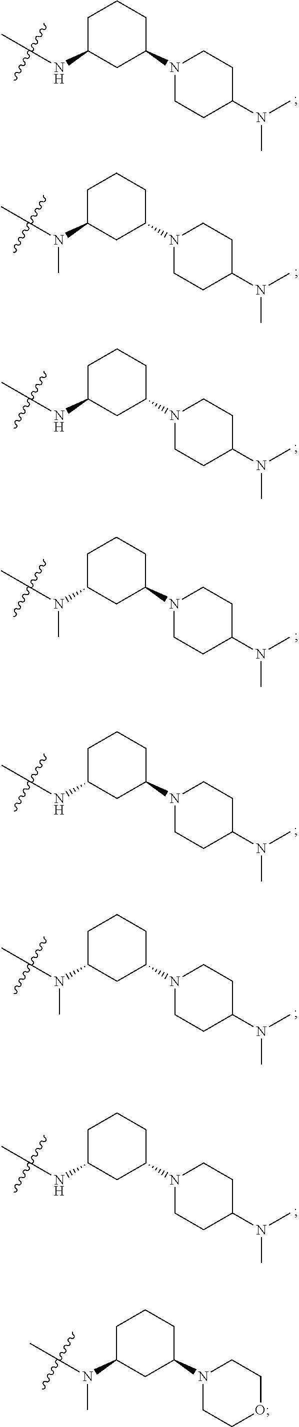

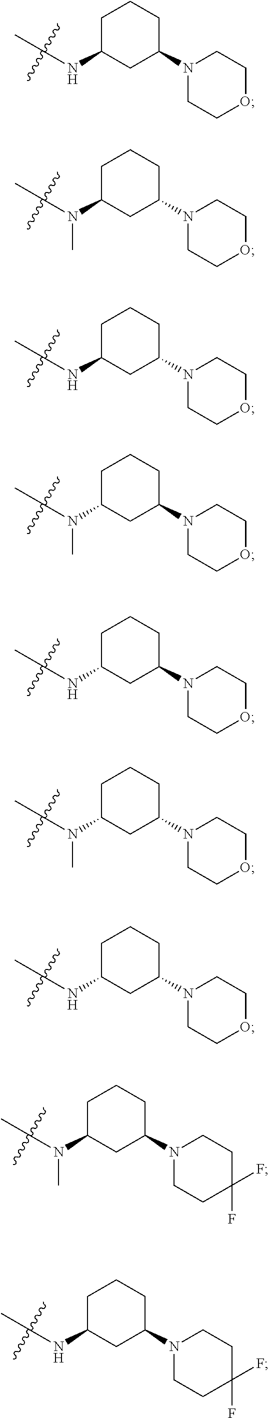

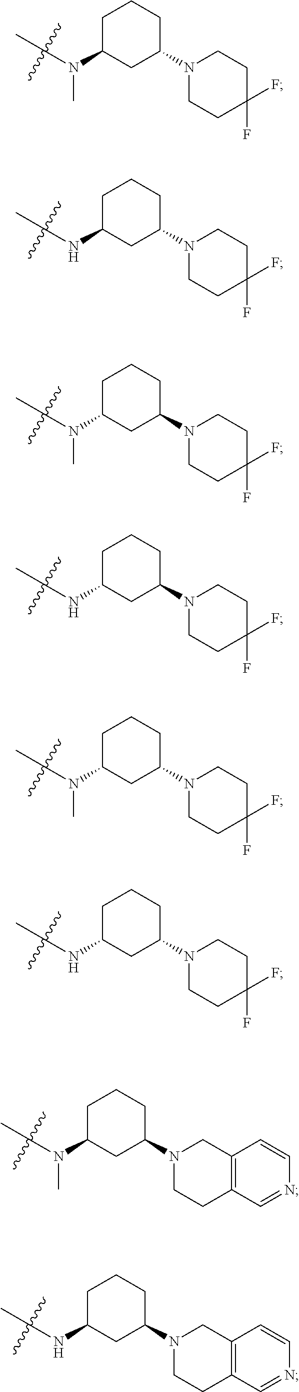

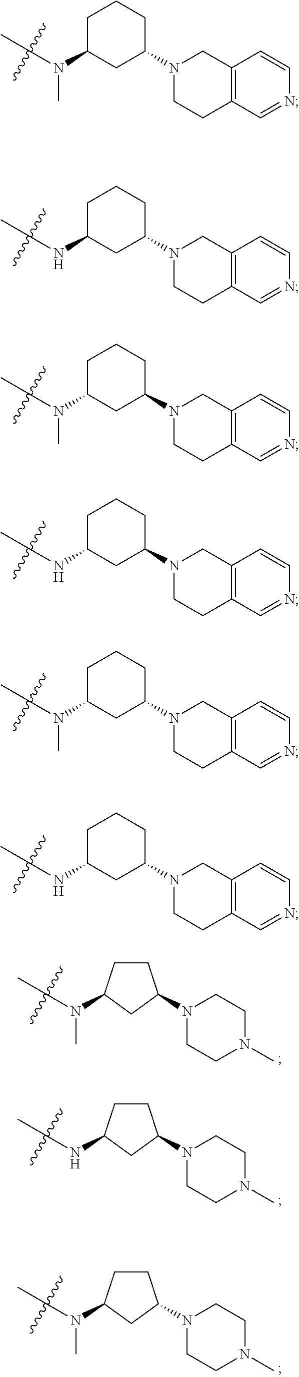

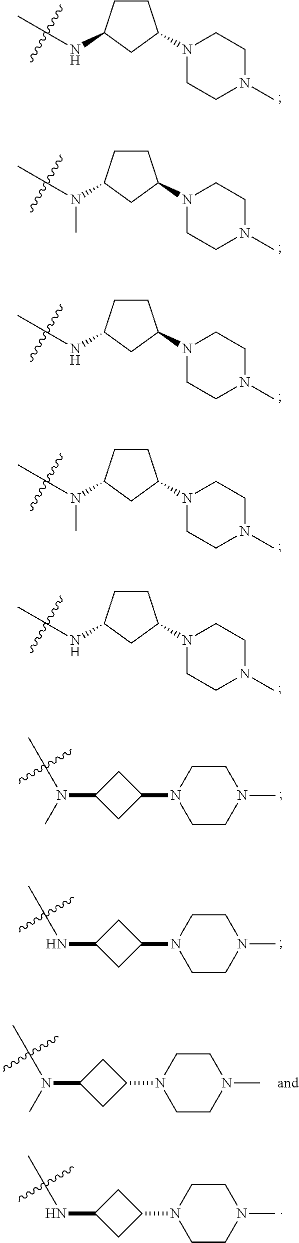

In certain embodiments of the compounds according to the present invention the substructure within general formula I represented by general formula AC II

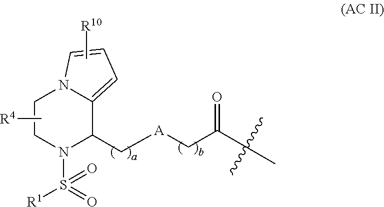

is selected from

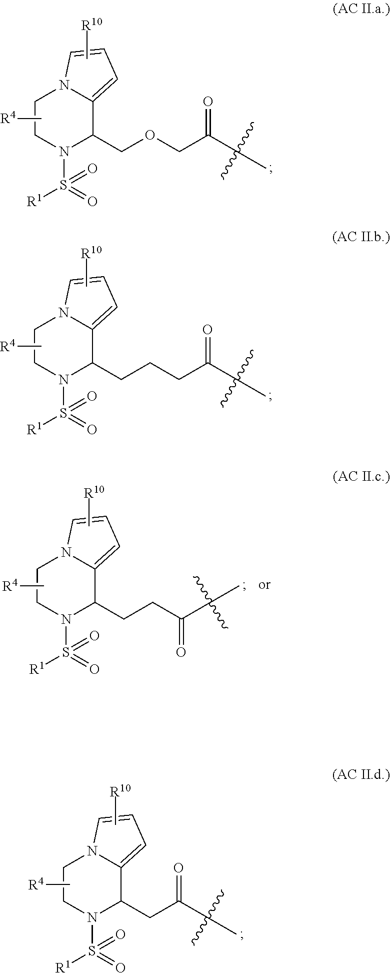

wherein R¹ may have any of the meanings as defined herein. In certain embodiments of the inventive compounds R⁴ and/or R¹⁰ are absent.

In certain embodiments of the compounds of the present invention, R¹ is selected from 2,6-dimethyl-4-methoxy-phenyl, 2-6-dichloro-3-methylphenyl, 3-trifluoromethyl-phenyl, 2-chlor-6-methyl-phenyl, 4-chlor-2,5-dimethylphenyl and 6-methoxy-naphthyl. In further embodiments of the compounds of the present invention, R¹ is selected from 2,6-dimethyl-4-methoxy-phenyl, 2-6-dichloro-3-methylphenyl, 2-chlor-6-methyl-phenyl or 4-chlor-2,5-dimethylphenyl. In specific embodiments of the compounds of the present invention, R¹ is 2,6-dimethyl-4-methoxy-phenyl. In other embodiments of the compounds of the present invention, R¹ is 6-methoxy-naphthyl.

Specific embodiments of the compounds according to the present invention are those listed in the following:
[SC-01] 2-[[2-[(4-Methoxy-2,6-dimethyl-phenyl)sulfonyl]-1,2,3,4-tetrahydro-pyrrolo[1,2-a]pyrazin-1-yl]-methoxy]-N-methyl-N-[(1S,3R)-3-(4-methyl-piperazin-1-yl)-cyclohexyl]-acetamide,
[SC-02] 2-[[2-[(2-Chloro-6-methyl-phenyl)sulfonyl]-1,2,3,4-tetrahydro-pyrrolo[1,2-a]pyrazin-1-yl]-methoxy]-N-methyl-N-[(1S,3R)-3-(4-methyl-piperazin-1-yl)-cyclohexyl]-acetamide,
[SC-03]3-[2-[(4-Chloro-2,5-dimethyl-phenyl)sulfonyl]-1,2,3,4-tetrahydro-pyrrolo[1,2-a]pyrazin-1-yl]-N-methyl-N-[(1S,3R)-3-(4-methyl-piperazin-1-yl)-cyclohexyl]-propionamide,
[SC-05] 2-[[2-[(4-Methoxy-2,6-dimethyl-phenyl)sulfonyl]-1,2,3,4-tetrahydro-pyrrolo[1,2-a]pyrazin-1-yl]-methoxy]-N-methyl-N-[4-(4-methyl-piperazin-1-yl)-cyclohexyl]-acetamide,
[SC-06] 2-[[2-[(4-Methoxy-2,6-dimethyl-phenyl)sulfonyl]-6-methyl-1,2,3,4-tetrahydro-pyrrolo[1,2-a]pyrazin-1-yl]-methoxy]-N-methyl-N-[(1S,3R)-3-(4-methyl-piperazin-1-yl)-cyclohexyl]-acetamide,
[CC-01] 2-[[2-[(4-Methoxy-2,6-dimethyl-phenyl)sulfonyl]-1,2,3,4-tetrahydro-pyrrolo[1,2-a]pyrazin-1-yl]-methoxy]-N-methyl-N-[cis-3-(4-methyl-piperazin-1-yl)-cyclohexyl]-acetamide,
[CC-02] 4-[2-[(4-Methoxy-2,6-dimethyl-phenyl)sulfonyl]-1,2,3,4-tetrahydro-pyrrolo[1,2-a]pyrazin-1-yl]-N-methyl-N-[cis-3-(4-methyl-piperazin-1-yl)-cyclohexyl]-butyramide,
[CC-03] 2-[[2-[(2,6-Dichloro-3-methyl-phenyl)sulfonyl]-1,2,3,4-tetrahydro-pyrrolo[1,2-a]pyrazin-1-yl]-methoxy]-N-methyl-N-[cis-3-(4-methyl-piperazin-1-yl)-cyclohexyl]-acetamide,
[CC-04] 2-[2-[(6-Methoxy-naphthalen-2-yl)sulfonyl]-1,2,3,4-tetrahydro-pyrrolo[1,2-a]pyrazin-1-yl]-N-methyl-N-[cis-3-(4-methyl-piperazin-1-yl)-cyclohexyl]-acetamide,
[CC-05] N-Methyl-N-[cis-3-(4-methyl-piperazin-1-yl)-cyclohexyl]-3-[2-[[3-(trifluoromethyl)phenyl]sulfonyl]-1,2,3,4-tetrahydro-pyrrolo[1,2-a]pyrazin-1-yl]-propionamide,
[CC-06] 2-[[2-[(4-Methoxy-2,6-dimethyl-phenyl)sulfonyl]-1,2,3,4-tetrahydro-pyrrolo[1,2-a]pyrazin-1-yl]-methoxy]-N-[cis-3-(4-methyl-piperazin-1-yl)-cyclohexyl]-acetamide,
[CC-07] 4-[2-[(4-Methoxy-2,6-dimethyl-phenyl)sulfonyl]-1,2,3,4-tetrahydro-pyrrolo[1,2-a]pyrazin-1-yl]-N-[cis-3-(4-methyl-piperazin-1-yl)-cyclohexyl]-butyramide,
[CC-08] 2-[[2-[(2,6-Dichloro-3-methyl-phenyl)sulfonyl]-1,2,3,4-tetrahydro-pyrrolo[1,2-a]pyrazin-1-yl]-methoxy]-N-[cis-3-(4-methyl-piperazin-1-yl)-cyclohexyl]-acetamide,
[CC-09] 2-[2-[(6-Methoxy-naphthalen-2-yl)sulfonyl]-1,2,3,4-tetrahydro-pyrrolo[1,2-a]pyrazin-1-yl]-N-[cis-3-(4-methyl-piperazin-1-yl)-cyclohexyl]-acetamide,
[CC-10] N-[cis-3-(4-Methyl-piperazin-1-yl)-cyclohexyl]-3-[2-[[3-(trifluoromethyl)phenyl]sulfonyl]-1,2,3,4-tetrahydro-pyrrolo[1,2-a]pyrazin-1-yl]-propionamide,
[CC-11] 2-[[2-[(4-Methoxy-2,6-dimethyl-phenyl)sulfonyl]-1,2,3,4-tetrahydro-pyrrolo[1,2-a]pyrazin-1-yl]-methoxy]-N-methyl-N-[trans-3-(4-methyl-piperazin-1-yl)-cyclohexyl]-acetamide,
[CC-12] 4-[2-[(4-Methoxy-2,6-dimethyl-phenyl)sulfonyl]-1,2,3,4-tetrahydro-pyrrolo[1,2-a]pyrazin-1-yl]-N-methyl-N-[trans-3-(4-methyl-piperazin-1-yl)-cyclohexyl]-butyramide,
[CC-13] 2-[[2-[(2,6-Dichloro-3-methyl-phenyl)sulfonyl]-1,2,3,4-tetrahydro-pyrrolo[1,2-a]pyrazin-1-yl]-methoxy]-N-methyl-N-[trans-3-(4-methyl-piperazin-1-yl)-cyclohexyl]-acetamide,
[CC-14] 2-[2-[(6-Methoxy-naphthalen-2-yl)sulfonyl]-1,2,3,4-tetrahydro-pyrrolo[1,2-a]pyrazin-1-yl]-N-methyl-N-[trans-3-(4-methyl-piperazin-1-yl)-cyclohexyl]-acetamide,
[CC-15] N-Methyl-N-[trans-3-(4-methyl-piperazin-1-yl)-cyclohexyl]-3-[2-[[3-(trifluoromethyl)phenyl]sulfonyl]-1,2,3,4-tetrahydro-pyrrolo[1,2-a]pyrazin-1-yl]-propionamide,
[CC-16] N-[cis-3-(4-Isopropyl-piperazin-1-yl)-cyclohexyl]-2-[[2-[(4-methoxy-2,6-dimethyl-phenyl)sulfonyl]-1,2,3,4-tetrahydro-pyrrolo[1,2-a]pyrazin-1-yl]-methoxy]-N-methyl-acetamide,
[CC-17] N-[cis-3-(4-Isopropyl-piperazin-1-yl)-cyclohexyl]-4-[2-[(4-methoxy-2,6-dimethyl-phenyl)sulfonyl]-1,2,3,4-tetrahydro-pyrrolo[1,2-a]pyrazin-1-yl]-N-methyl-butyramide,
[CC-18] 2-[[2-[(2,6-Dichloro-3-methyl-phenyl)sulfonyl]-1,2,3,4-tetrahydro-pyrrolo[1,2-a]pyrazin-1-yl]-methoxy]-N-[cis-3-(4-isopropyl-piperazin-1-yl)-cyclohexyl]-N-methyl-acetamide,
[CC-19] N-[cis-3-(4-Isopropyl-piperazin-1-yl)-cyclohexyl]-2-[2-[(6-methoxy-naphthalen-2-yl)sulfonyl]-1,2,3,4-tetrahydro-pyrrolo[1,2-a]pyrazin-1-yl]-N-methyl-acetamide,
[CC-20] N-[cis-3-(4-Isopropyl-piperazin-1-yl)-cyclohexyl]-N-methyl-3-[2-[[3-(trifluoromethyl)phenyl]sulfonyl]-1,2,3,4-tetrahydro-pyrrolo[1,2-a]pyrazin-1-yl]-propionamide,
[CC-21] N-[cis-3-(4-Dimethylamino-piperidin-1-yl)-cyclohexyl]-2-[[2-[(4-methoxy-2,6-dimethyl-phenyl)sulfonyl]-1,2,3,4-tetrahydro-pyrrolo[1,2-a]pyrazin-1-yl]-methoxy]-N-methyl-acetamide,
[CC-22] N-[cis-3-(4-Dimethylamino-piperidin-1-yl)-cyclohexyl]-4-[2-[(4-methoxy-2,6-dimethyl-phenyl)sulfonyl]-1,2,3,4-tetrahydro-pyrrolo[1,2-a]pyrazin-1-yl]-N-methyl-butyramide,
[CC-23] 2-[[2-[(2,6-Dichloro-3-methyl-phenyl)sulfonyl]-1,2,3,4-tetrahydro-pyrrolo[1,2-a]pyrazin-1-yl]-methoxy]-N-[cis-3-(4-dimethylamino-piperidin-1-yl)-cyclohexyl]-N-methyl-acetamide,
[CC-24] N-[cis-3-(4-Dimethylamino-piperidin-1-yl)-cyclohexyl]-2-[2-[(6-methoxy-naphthalen-2-yl)sulfonyl]-1,2,3,4-tetrahydro-pyrrolo[1,2-a]pyrazin-1-yl]-N-methyl-acetamide,
[CC-25] N-[cis-3-(4-Dimethylamino-piperidin-1-yl)-cyclohexyl]-N-methyl-3-[2-[[3-(trifluoromethyl)phenyl]sulfonyl]-1,2,3,4-tetrahydro-pyrrolo[1,2-a]pyrazin-1-yl]-propionamide,
[CC-26] 2-[[2-[(4-Methoxy-2,6-dimethyl-phenyl)sulfonyl]-1,2,3,4-tetrahydro-pyrrolo[1,2-a]pyrazin-1-yl]-methoxy]-N-methyl-N-[cis-3-morpholin-4-yl-cyclohexyl]-acetamide,
[CC-27]4-[2-[(4-Methoxy-2,6-dimethyl-phenyl)sulfonyl]-1,2,3,4-tetrahydro-pyrrolo[1,2-a]pyrazin-1-yl]-N-methyl-N-[cis-3-morpholin-4-yl-cyclohexyl]-butyramide,
[CC-28] 2-[[2-[(2,6-Dichloro-3-methyl-phenyl)sulfonyl]-1,2,3,4-tetrahydro-pyrrolo[1,2-a]pyrazin-1-yl]-methoxy]-N-methyl-N-[cis-3-morpholin-4-yl-cyclohexyl]-acetamide,
[CC-29] 2-[2-[(6-Methoxy-naphthalen-2-yl)sulfonyl]-1,2,3,4-tetrahydro-pyrrolo[1,2-a]pyrazin-1-yl]-N-methyl-N-[cis-3-morpholin-4-yl-cyclohexyl]-acetamide,
[CC-30] N-Methyl-N-[cis-3-morpholin-4-yl-cyclohexyl]-3-[2-[[3-(trifluoromethyl)phenyl]sulfonyl]-1,2,3,4-tetrahydro-pyrrolo[1,2-a]pyrazin-1-yl]-propionamide,
[CC-31] N-[cis-3-(4,4-Difluoro-piperidin-1-yl)-cyclohexyl]-2-[[2-[(4-methoxy-2,6-dimethyl-phenyl)sulfonyl]-1,2,3,4-tetrahydro-pyrrolo[1,2-a]pyrazin-1-yl]-methoxy]-N-methyl-acetamide,
[CC-32] N-[cis-3-(4,4-Difluoro-piperidin-1-yl)-cyclohexyl]-4-[2-[(4-methoxy-2,6-dimethyl-phenyl)sulfonyl]-1,2,3,4-tetrahydro-pyrrolo[1,2-a]pyrazin-1-yl]-N-methyl-butyramide,
[CC-33] 2-[[2-[(2,6-Dichloro-3-methyl-phenyl)sulfonyl]-1,2,3,4-tetrahydro-pyrrolo[1,2-a]pyrazin-1-yl]-methoxy]-N-[cis-3-(4,4-difluoro-piperidin-1-yl)-cyclohexyl]-N-methyl-acetamide,
[CC-34] N-[cis-3-(4,4-Difluoro-piperidin-1-yl)-cyclohexyl]-2-[2-[(6-methoxy-naphthalen-2-yl)sulfonyl]-1,2,3,4-tetrahydro-pyrrolo[1,2-a]pyrazin-1-yl]-N-methyl-acetamide,
[CC-35] N-[cis-3-(4,4-Difluoro-piperidin-1-yl)-cyclohexyl]-N-methyl-3-[2-[[3-(trifluoromethyl)phenyl]sulfonyl]-1,2,3,4-tetrahydro-pyrrolo[1,2-a]pyrazin-1-yl]-propionamide,
[CC-36] 2-[[2-[(4-Methoxy-2,6-dimethyl-phenyl)sulfonyl]-1,2,3,4-tetrahydro-pyrrolo[1,2-a]pyrazin-1-yl]-methoxy]-N-methyl-N-[cis-3-(1,2,3,4-tetrahydro-[2,6]naphthyridin-2-yl)-cyclohexyl]-acetamide,
[CC-37] 4-[2-[(4-Methoxy-2,6-dimethyl-phenyl)sulfonyl]-1,2,3,4-tetrahydro-pyrrolo[1,2-a]pyrazin-1-yl]-N-methyl-N-[cis-3-(1,2,3,4-tetrahydro-[2,6]naphthyridin-2-yl)-cyclohexyl]-butyramide,
[CC-38] 2-[[2-[(2,6-Dichloro-3-methyl-phenyl)sulfonyl]-1,2,3,4-tetrahydro-pyrrolo[1,2-a]pyrazin-1-yl]-methoxy]-N-methyl-N-[cis-3-(1,2,3,4-tetrahydro-[2,6]naphthyridin-2-yl)-cyclohexyl]-acetamide,
[CC-39] 2-[2-[(6-Methoxy-naphthalen-2-yl)sulfonyl]-1,2,3,4-tetrahydro-pyrrolo[1,2-a]pyrazin-1-yl]-N-methyl-N-[cis-3-(1,2,3,4-tetrahydro-[2,6]naphthyridin-2-yl)-cyclohexyl]-acetamide,
[CC-40] N-Methyl-N-[cis-3-(1,2,3,4-tetrahydro-[2,6]naphthyridin-2-yl)-cyclohexyl]-3-[2-[[3-(trifluoromethyl)phenyl]sulfonyl]-1,2,3,4-tetrahydro-pyrrolo[1,2-a]pyrazin-1-yl]-propionamide,
[CC-50] N-[3-(4-Isopropyl-piperazin-1-yl)-cyclohexyl]-2-[[2-[(4-methoxy-2,6-dimethyl-phenyl)sulfonyl]-1,2,3,4-tetrahydro-pyrrolo[1,2-a]pyrazin-1-yl]-methoxy]-N-methyl-acetamide,
[CC-51] N-[3-(4-Isopropyl-piperazin-1-yl)-cyclohexyl]-4-[2-[(4-methoxy-2,6-dimethyl-phenyl)sulfonyl]-1,2,3,4-tetrahydro-pyrrolo[1,2-a]pyrazin-1-yl]-N-methyl-butyramide,
[CC-52] N-[3-(4-Isopropyl-piperazin-1-yl)-cyclohexyl]-2-[2-[(6-methoxy-naphthalen-2-yl)sulfonyl]-1,2,3,4-tetrahydro-pyrrolo[1,2-a]pyrazin-1-yl]-N-methyl-acetamide,
[CC-53] N-[3-(4-Isopropyl-piperazin-1-yl)-cyclohexyl]-N-methyl-3-[2-[[3-(trifluoromethyl)phenyl]sulfonyl]-1,2,3,4-tetrahydro-pyrrolo[1,2-a]pyrazin-1-yl]-propionamide,
[CC-54] 2-[[2-[(4-Methoxy-2,6-dimethyl-phenyl)sulfonyl]-1,2,3,4-tetrahydro-pyrrolo[1,2-a]pyrazin-1-yl]-methoxy]-N-methyl-N-(3-morpholin-4-yl-cyclohexyl)-acetamide,
[CC-55] 4-[2-[(4-Methoxy-2,6-dimethyl-phenyl)sulfonyl]-1,2,3,4-tetrahydro-pyrrolo[1,2-a]pyrazin-1-yl]-N-methyl-N-(3-morpholin-4-yl-cyclohexyl)-butyramide,
[CC-56] 2-[2-[(6-Methoxy-naphthalen-2-yl)sulfonyl]-1,2,3,4-tetrahydro-pyrrolo[1,2-a]pyrazin-1-yl]-N-methyl-N-(3-morpholin-4-yl-cyclohexyl)-acetamide,
[CC-57] N-Methyl-N-(3-morpholin-4-yl-cyclohexyl)-3-[2-[[3-(trifluoromethyl)phenyl]sulfonyl]-1,2,3,4-tetrahydro-pyrrolo[1,2-a]pyrazin-1-yl]-propionamide,
[CC-58] 2-[[2-[(4-Methoxy-2,6-dimethyl-phenyl)sulfonyl]-1,2,3,4-tetrahydro-pyrrolo[1,2-a]pyrazin-1-yl]-methoxy]-N-methyl-N-[3-(4-methyl-piperazin-1-yl)-cyclobutyl]-acetamide,
[CC-59] 4-[2-[(4-Methoxy-2,6-dimethyl-phenyl)sulfonyl]-1,2,3,4-tetrahydro-pyrrolo[1,2-a]pyrazin-1-yl]-N-methyl-N-[3-(4-methyl-piperazin-1-yl)-cyclobutyl]-butyramide,
[CC-60] 2-[2-[(6-Methoxy-naphthalen-2-yl)sulfonyl]-1,2,3,4-tetrahydro-pyrrolo[1,2-a]pyrazin-1-yl]-N-methyl-N-[3-(4-methyl-piperazin-1-yl)-cyclobutyl]-acetamide,
[CC-61] N-Methyl-N-[3-(4-methyl-piperazin-1-yl)-cyclobutyl]-3-[2-[[3-(trifluoromethyl)phenyl]sulfonyl]-1,2,3,4-tetrahydro-pyrrolo[1,2-a]pyrazin-1-yl]-propionamide,
   optionally in the form of an individual enantiomer or an individual diastereomer, the racemate, the enantiomers, the diastereomers, mixtures of enantiomers or diastereomers, each in the form of their bases and/or physiologically compatible salts, in particular hydrochloride salts.

The numbering of the individual embodiments of the compounds according to the invention used above is retained in the following explanations of the present invention, particularly in the description of the examples.

According to one aspect of the present invention the compounds according to the invention preferably have an antagonistic action on the human B1R receptor or the B1R receptor of the rat. In a preferred embodiment of the invention the compounds according to the invention have an antagonistic action on both the human B1R receptor (hB1R) and on the B1R receptor of the rat (rB1R).

In a preferred embodiment of the present invention the compounds according to the invention exhibit at least 15%, 25%, 50%, 70%, 80% or 90% inhibition on the human B1R receptor and/or on the B1R receptor of the rat in the FLIPR assay at a concentration of 10 μM. Most particularly preferred are compounds which exhibit at least 70%, in particular at least 80% and particularly preferably at least 90% inhibition on the human B1R receptor and on the B1R receptor of the rat at a concentration of 10 μM.

The agonistic or antagonistic action of substances can be quantified on the bradykinin 1 receptor (B1R) of the human and rat species with ectopically expressing cell lines (CHO K1 cells) and with the aid of a Ca2+-sensitive dye (Fluo-4) using a fluorescent imaging plate reader (FLIPR). The value in % activation is based on the Ca2+ signal after addition of Lys-Des-Arg9 bradykinin (0.5 nM) or Des-Arg9 bradykinin (100 nM). Antagonists lead to a suppression of the Ca2+ influx following administration of the agonist. The % inhibition in comparison with the maximum achievable inhibition is indicated.

A further aspect of the present invention is a medicament comprising at least one compound as described above and at least one pharmaceutically acceptable excipient.

In yet a further aspect the present provides a compound for the use in the treatment, in particular acute pain, visceral pain, neuropathic pain, chronic pain and/or inflammatory pain; migraine; diabetes; diseases of the respiratory tract; inflammatory bowel diseases; neurological diseases; inflammations of the skin; rheumatic diseases; septic shock; reperfusion syndrome; obesity, and/or as an angiogenesis inhibitor, wherein the compound is one of the inventive compounds as defined herein.

The medicaments according to the invention optionally contain, in addition to at least one tetrahydropyrrolopyrazine according to the invention, at least one suitable additive and/or auxiliary substance, including carrier materials, fillers, solvents, diluents, dyes and/or binders, and can be administered as liquid dosage forms in the form of injection solutions, drops or juices, as semi-solid dosage forms in the form of granules, tablets, pellets, patches, capsules, plasters/spray plasters or aerosols. The choice of auxiliary substances, etc., and the amounts thereof to use depend on whether the medicinal product is to be administered by oral, peroral, parenteral, intravenous, intraperitoneal, intradermal, intramuscular, nasal, buccal, rectal or topical means, for example on the skin, mucous membranes or in the eyes. Preparations in the form of tablets, pastilles, capsules, granules, drops, juices and syrups are suitable for oral administration; solutions, suspensions, easily reconstitutable dry preparations and sprays are suitable for parenteral, topical and inhalative administration. Tetrahydropyrrolopyrazines according to the invention in a depot formulation, in dissolved form or in a plaster, optionally with addition of agents promoting skin penetration, are suitable preparations for percutaneous administration. Preparation forms suitable for oral or percutaneous administration can deliver the tetrahydropyrrolopyrazines according to the invention on a delayed release basis. The tetrahydropyrrolopyrazines according to the invention can also be used in parenteral long-term depot forms, such as implants or implanted pumps, for example. Other additional active ingredients known to the person skilled in the art can be added in principle to the medicinal products according to the invention.

The amount of active ingredient to be administered to the patient varies according to the weight of the patient, the type of administration, the indication and the severity of the illness. 0.00005 to 50 mg/kg, preferably 0.01 to 5 mg/kg, of at least one tetrahydropyrrolopyrazine according to the invention are conventionally administered. A preferred form of the medicinal product contains a tetrahydropyrrolopyrazine according to the invention as a pure diastereomer and/or enantiomer, as a racemate or as a non-equimolar or equimolar mixture of diastereomers and/or enantiomers.

### General Process for the Preparation of the Substituted Tetrahydropyrrolopyrazine Derivatives

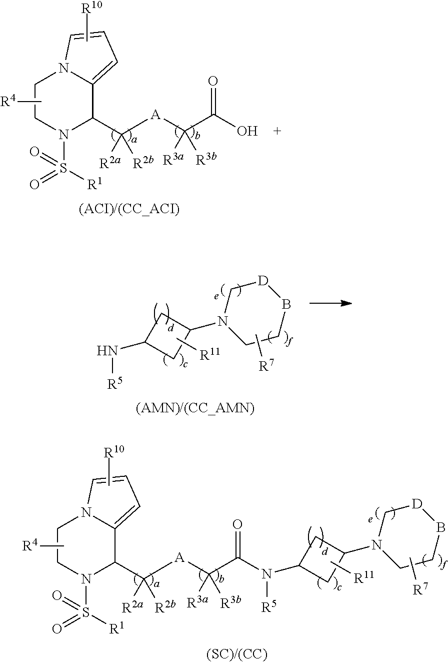

The carboxylic acids (ACI) or (CC_ACI) and amines (AMN) or (CC_AMN) are reacted in an amide forming reaction to give the desired tetrahydropyrrolopyrazine derivatives (SC/CC)) according to the invention.

In such a reaction compounds of the general formula (ACI) or (CC_ACI) are reacted in at least one solvent, preferably chosen from the group consisting of methylene chloride, acetonitrile, dimethylformamide, diethyl ether, dioxane and tetrahydrofuran, with amines (AMN) or (CC_AMN), with the addition of at least one coupling reagent, preferably chosen from the group consisting of carbonyldiimidazole (CDI), 2-chloro-1-methylpyridinium iodide (Mukaiyama reagent), N-(3-dimethylaminopropyl)-N′-ethylcarbodiimide (EDCl), O-(benzotriazol-1-yl)-N,N,N′,N′-tetramethyluronium tetrafluoroborate (TBTU), N,N′-dicyclohexylcarbodiimide (DCC), O-(7-Azabenzotriazol-1-yl)N,N,N′,N′-tetramethyluronium hexafluorophosphate (HATU) and 1-benzotriazolyloxy-tris-(dimethylamino)-phosphonium hexafluorophosphate (BOP), optionally in the presence of at least one inorganic base, preferably chosen from the group consisting of potassium carbonate and caesium carbonate, or an organic base, preferably chosen from the group consisting of triethylamine, diisopropylethylamine and pyridine, and optionally with the addition of 4-(dimethylamino)pyridine or 1-hydroxybenzotriazole, to give compounds with the general formula (SC) or (CC).

The carboxylic acids (ACI) or (CC_ACI) employed can be synthesised according to the methods described below.

The amines (AMN) or (CC_AMN) can be synthesized according to or in analogy to literature procedures, see e.g. WO 2009/021944, WO2010/017850.

### General Synthesis of Carboxylic Acids (ACI) or (CC_ACI)

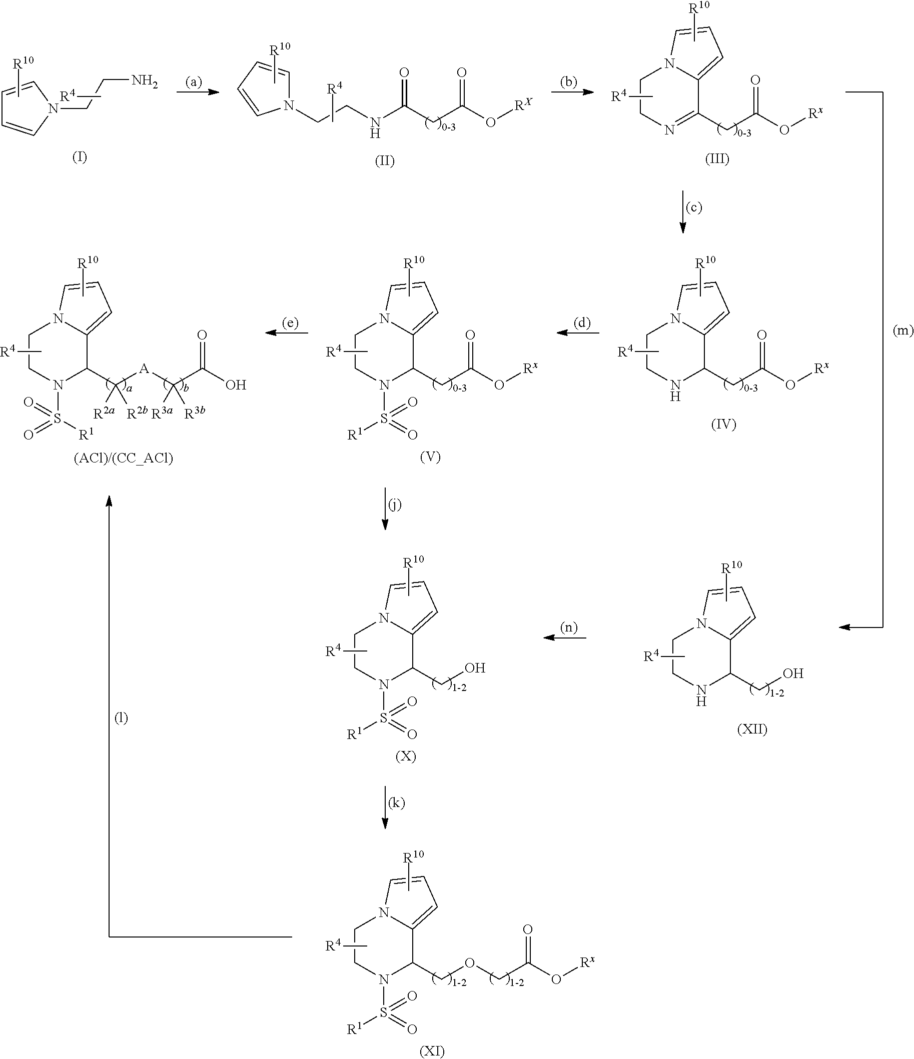

In step (a), the acylation of amines of the general formula (I) with an oxalic acid monoester, a malonic acid monoester, a succinic acid monoester or a glutaric acid monoester is performed in at least one solvent, preferably chosen from the group consisting of methylene chloride, acetonitrile, dimethylformamide, diethyl ether, dioxane and tetrahydrofuran, with the addition of at least one coupling reagent, preferably chosen from the group consisting of carbonyldiimidazole (CDI), 2-chloro-1-methylpyridinium iodide (Mukaiyama reagent), N-(3-dimethylaminopropyl)-N′-ethylcarbodiimide (EDCl), O-(benzotriazol-1-yl)-N,N,N′,N′-tetramethyluronium tetrafluoroborate (TBTU), N,N′-dicyclohexylcarbodiimide (DCC), O-(7-Azabenzotriazol-1-yl)N,N,N′,N′-tetramethyluronium hexafluorophosphate (HATU) and 1-benzotriazolyloxy-tris-(dimethylamino)-phosphonium hexafluorophosphate (BOP), optionally in the presence of at least one inorganic base, preferably chosen from the group consisting of potassium carbonate and caesium carbonate, or an organic base, preferably chosen from the group consisting of triethylamine, diisopropylethylamine and pyridine, and optionally with the addition of 4-(dimethylamino)pyridine or 1-hydroxybenzotriazole, to give compounds with the general formula (II).

The corresponding acid chloride monoesters can also be used in place of the oxalic acid monoesters, the malonic acid monoesters, the succinic acid monoesters and the glutaric acid monoesters. In step (a), amines of the general formula (I) are reacted in at least one solvent, preferably chosen from the group consisting of methylene chloride, acetonitrile, dimethylformamide, diethyl ether, dioxane, tetrahydrofuran, methanol, ethanol and isopropanol, with an appropriate acid chloride, in the presence of at least one inorganic base, preferably chosen from the group consisting of potassium carbonate and caesium carbonate, or an organic base, preferably chosen from the group consisting of triethylamine, diisopropylethylamine and pyridine, and optionally with the addition of 4-(dimethylamino)pyridine at temperatures of from preferably −15° C. to 50° C. to give compounds of the general formula (II).

In step (b) the cyclisation of compounds of the general formula (II) to cyclic imines of the general formula (III) is performed by reacting with POCl₃ in a suitable solvent or mixtures thereof, preferably chosen from the group consisting of benzene, toluene, ethanol or water; or by reacting with/in a suitable acid, preferably chosen from the group consisting of polyphosphoric acid and trifluoroacetic acid at temperatures of from preferably −50° C. to 250° C. to give compounds of the general formula (III).

In step (c) the reduction of cyclic imines of the general formula (III) can be performed using a suitable reducing agent, such as for example NaBH₄, in a suitable solvent or mixtures thereof, preferably chosen from the group consisting of ethanol, methanol or water; or by hydrogenolysis in the presence of a suitable catalyst, preferably chosen from the group consisting of Pd/C and Pd on BaSO₄, in a suitable solvent, such as for example ethanol at temperatures of from preferably −70° C. to 100° C. to give compounds of the general formula (IV).

In step (d) amines of the general formula (IV) are reacted in at least one solvent, preferably chosen from the group consisting of methylene chloride, acetonitrile, dimethylformamide, diethyl ether, dioxane, tetrahydrofuran, methanol, ethanol and isopropanol, with sulfonyl chlorides, in the presence of at least one inorganic base, preferably chosen from the group consisting of potassium carbonate and caesium carbonate, or an organic base, preferably chosen from the group consisting of triethylamine, diisopropylethylamine and pyridine, and optionally with the addition of 4-(dimethylamino)pyridine, at temperatures of from preferably −15° C. to 50° C. to give compounds with the general formula (V).

In step (e) compounds of the general formula (V) are reacted in at least one solvent, preferably chosen from the group consisting of water, methanol, ethanol, isopropanol, diethyl ether, tetrahydrofuran, toluene, acetonitrile, dimethylformamide, dioxane and dimethylsulfoxide, with an inorganic base, preferably chosen from the group consisting of lithium hydroxide, sodium hydroxide, potassium hydroxide, potassium tert-butanolate, sodium carbonate, sodium hydrogen carbonate, potassium carbonate, lithium propanethiolate and sodium phenylselenolate, optionally with the addition of HMPA or lithium chloride, or with a Lewis acid, preferably chosen from the group consisting of trimethylsilyl chloride, boron tribromide and aluminium trichloride, or with an organic acids, such as trifluoroacetic acid, or with an aqueous inorganic acid, such as hydrochloric acid, optionally with the addition of thiolene, sodium iodide or lithium chloride, at temperatures of from preferably 0° C. to 100° C. to give compounds of the general formula (ACI) or (CC_ACI).

In step (j) compounds of the general formula (V) are reduced in at least one solvent, preferably chosen from the group consisting of benzene, dimethoxy ethane, diethyl ether, toluene, tetrahydrofuran, water, hexane, dichloromethane, methanol and ethanol, in the presence of at least one reducing agent, preferably chosen from the group consisting of LiBH₄, BH₃-Me₂S, Zn(BH₄)₂, NaBH₄, diisobutylaluminium hydride (DIBAL-H) and lithium aluminium hydride (LAH), optionally in the presence of boronic esters, at temperatures of from preferably −100° C. to 150° C. to give compounds of the general formula (X).

Alternatively, compounds of the general formula (X) can be prepared via compounds of the general formula (XII), starting from compounds of the general formula (III). To achieve this in in step (m) compounds of the general formula (III) are reduced in at least one solvent, preferably chosen from the group consisting of benzene, dimethoxy ethane, diethyl ether, toluene, tetrahydrofuran, water, hexane, dichloromethane, methanol and ethanol, in the presence of at least one reducing agent, preferably chosen from the group consisting of LiBH₄, BH₃-Me₂S, Zn(BH₄)₂, NaBH₄, diisobutylaluminium hydride (DIBAL-H) and lithium aluminium hydride (LAH), optionally in the presence of boronic esters, at temperatures of from preferably −100° C. to 150° C. to give compounds of the general formula (XII). This is followed by step (n), in which compounds of the general formula (XII) are reacted in at least one solvent, preferably chosen from the group consisting of methylene chloride, acetonitrile, dimethylformamide, diethyl ether, dioxane, tetrahydrofuran, methanol, ethanol and isopropanol, with sulfonyl chlorides, in the presence of at least one inorganic base, preferably chosen from the group consisting of potassium carbonate and caesium carbonate, or an organic base, preferably chosen from the group consisting of triethylamine, diisopropylethylamine and pyridine, and optionally with the addition of 4-(dimethylamino)pyridine, at temperatures of from preferably −15° C. to 50° C. to give compounds with the general formula (X).

In step (k) compounds of the general formula (X) are reacted with halogenated ester in a phase transfer reaction in a suitable solvent or mixtures thereof, preferably chosen from the group consisting of toluene, benzene, dichloromethane, xylene and water, in the presence of a suitable phase transfer catalyst, preferably chosen from the group consisting of tetrabutylammonium chloride, tetrabutylammonium bromide and tetrabutylammonium hydrogen sulfate, and in the presence of an inorganic base, such as potassium hydroxide, sodium hydroxide, sodium carbonate, sodium hydrogen carbonate and potassium carbonate, at temperatures of from preferably −50° C. to 150° C. to give compounds of the general formula (XI). Alternatively, compounds of the general formula (X) are reacted with halogenated ester in at least one suitable organic solvent, preferably chosen from the group consisting of as dichloromethane, tetrahydrofuran, dimethylformamide and diethylether, in the presence of an organic or inorganic base, conventional inorganic bases being metal alcoholates such as sodium methanolate, sodium ethanolate, potassium tert-butylate, lithium or sodium bases such as lithium diisopropylamide, butyl lithium, tert-butyl lithium, sodium methylate, or metal hydrides such as potassium hydride, lithium hydride, sodium hydride, conventional organic bases being for example diisopropyl ethylamine and triethylamine, at temperatures of from preferably −100° C. to 100° C. to give compounds of the general formula (XI).

In step (I) compounds of the general formula (XI) are reacted in at least one solvent, preferably chosen from the group consisting of water, methanol, ethanol, isopropanol, diethyl ether, tetrahydrofuran, toluene, acetonitrile, dimethylformamide, dioxane and dimethylsulfoxide, with an inorganic base, preferably chosen from the group consisting of lithium hydroxide, sodium hydroxide, potassium hydroxide, potassium tert-butanolate, sodium carbonate, sodium hydrogen carbonate, potassium carbonate, lithium propanethiolate and sodium phenylselenolate, optionally with the addition of HMPA or lithium chloride, or with a Lewis acid, preferably chosen from the group consisting of trimethylsilyl chloride, boron tribromide and aluminium trichloride, or with an organic acids, such as trifluoroacetic acid, or with an aqueous inorganic acid, such as hydrochloric acid, optionally with the addition of thiolene, sodium iodide or lithium chloride, at temperatures of from preferably 0° C. to 100° C. to give compounds of the general formula (ACI) or (CC_ACI).

### Pharmacological Investigations

### 1. Functional Investigation on the Bradykinin 1 Receptor (B1R)

The agonistic or antagonistic action of substances can be determined on the bradykinin 1 receptor (B1R) of humans and rats by means of a cell-based fluorescent calcium-mobilization assay. According to this assay agonist-induced increase of intracellular free Ca²⁺ is quantified by means of a Ca²⁺-sensitive dye (Fluo-4 type, Molecular Probes Europe BV, Leiden, Netherlands), in a Fluorescent Imaging Plate Reader (FLIPR, Molecular Devices, Sunnyvale, USA) and/or the Novostar (BMG Labtech GmbH, Offenburg, Germany).

### Method:

Chinese hamster ovary cells (CHO K1 cells), which are stably transfected with the human B1R gene (hB1R cells), or with the B1R gene of rats (rB1R cells) are used. For functional investigations these cells are seeded into black 96-well plates with a clear bottom (BD Biosciences, Heidelberg, Germany or Greiner, Frickenhausen, Germany) in a density of 20,000-35,000 cells/well. The cells are incubated overnight in a humidified atmosphere at 37° C. and 5% CO₂ in culture medium (hB1R cells: Nutrient Mixture Ham's F12, Gibco Invitrogen GmbH, Karlsruhe, Germany or DMEM, Sigma-Aldrich, Taufkirchen, Germany; rB1R cells: D-MEM/F12, Gibco Invitrogen GmbH, Karlsruhe, Germany) with 10 vol. % FBS (foetal bovine serum, Gibco Invitrogen GmbH, Karlsruhe, Germany). On the following day the cells are loaded with the Ca²⁺-sensitive dye Fluo-4-AM (Molecular Probes Europe BV, Leiden, Netherlands):

Method A: The medium of the cells is removed and cell plates are incubated with loading solution, which contains 2.13 μM Fluo-4-AM, 2.5 mM probenecid (Sigma-Aldrich, Taufkirchen, Germany), and 10 mM HEPES (Sigma-Aldrich, Taufkirchen, Germany) in HBSS buffer (Hank's buffered saline solution, Gibco Invitrogen GmbH, Karlsruhe, Germany) for 60 minutes at 37° C. After the plates are washed twice with HBSS buffer, HBSS buffer supplemented 0.1% BSA (bovine serum albumin; Sigma-Aldrich, Taufkirchen, Germany), 5.6 mM glucose and 0.05% gelatine (Merck KGaA, Darmstadt, Germany) is added. Cell plates are incubated for at least 20 minutes in the dark at room temperature before they are used for the Ca²⁺ measurement in the FLIPR or Novostar.
Method B: The plates are washed with buffer A (15 mM HEPES, 80 mM NaCl, 5 mM KCl, 1.2 mM CaCl₂, 0.7 mM MgSO₄, 2 mg/ml glucose, 2.5 mM probenecid) and subsequently loaded with buffer A containing 2.4 μM Fluo-4-AM and 0.025% pluronic F127 (Sigma-Aldrich, Taufkirchen, Germany) for 60 minutes at 37° C. Cell plates are washed twice with buffer A. Then, buffer A supplemented with 0.05% BSA and 0.05% gelatine is added and cell plates are incubated in the dark at room temperature for at least 20 minutes before measurement in the FLIPR or Novostar is started.

### Fluorescence Assay:

The Ca²⁺-dependent fluorescence is measured both before and after the addition of substances (λₑₓ=488 nm, λₑₘ=540 nm). For quantification of the effect the highest fluorescence intensity (FC, fluorescence counts) over time is given.

The FLIPR protocol consists of two substance additions, done within the instrument while continuously monitoring the Ca²⁺-dependent fluorescence at 540 nm. When the Novostar was used, one addition had to be done outside the instrument. First of all test substances (10 μM) are pipetted onto the cells and the intracellular Ca²⁺ rise is compared with the control (hB1R: Lys-Des-Arg9-bradykinin >=50 nM; rB1R: Des-Arg9-bradykinin >=1 μM). This gives the result in % activation referred to the Ca²⁺ signal after addition of Lys-Des-Arg9-bradykinin (>=50 nM), or Des-Arg9-bradykinin (>=1 μM), respectively.

After incubation of test substances for 6-20 minutes the agonists Lys-Des-Arg9-bradykinin (hB1R) and Des-Arg⁹-bradykinin (rB1R) are applied in the EC₈₀ concentration and the increase of Ca²⁺ is likewise determined.

Antagonists lead to a suppression of the Ca²⁺ increase. The % inhibition compared to the maximum achievable inhibition is calculated.

The compounds preferably have a B1R-antagonistic action on the human receptor and/or on the rat receptor.

### EXAMPLES

The invention will be described in further detail by the following examples, without limiting the scope of the invention.

### LIST OF ABBREVIATIONS

min=minute(s)
Boc=tert-butoxycarbonyl
Cbz=benzyloxycarbonyl
conc.=concentrated
d=day(s)
DCM=dichloromethane
DIPEA=diisopropylethylamine
DMF=dimethylformamide
DMSO=dimethylsulfoxide
EDCl=N-(3-dimethylaminopropyl)-N′-ethylcarbodiimide hydrochloride
eq. equiv.=equivalent(s)
h=hour(s)
LAH=lithium aluminium hydride
HATU=O-(7-Azabenzotriazol-1-yl)N,N,N′,N′-tetramethyluronium hexafluorophosphate
M=molar
HOBt=1-hydroxybenzotriazole
MeOH=methanol
org.=organic
R.t.=retention time
RT=room temperature
sat.=saturated
TBACl=tetra-n-butyl ammonium chloride
TEA=triethylamine
TFA=trifluoroacetic acid
THF=tetrahydrofuran
t.l.c.=thin layer chromatography

The chemicals and solvents employed were obtained commercially from the conventional suppliers (Acros, Aldrich, Fluka, Lancaster, Maybridge, TCI, Fluorochem, Tyger, ABCR, Fulcrum, FrontierScientific, Milestone etc.). The reactions were carried out in some cases under inert gas (nitrogen). The yields of the compounds prepared are not optimized. The mixing ratios of solvents are always stated in the volume/volume ratio. The equivalent amounts of reagents employed and the amounts of solvent and reaction temperatures and times can vary slightly between different reactions carried out by the same method. The working up and purification methods were adapted according to the characteristic properties of the compounds.

### Synthesis of Example Compounds

### A) Synthesis of Single Compounds (SC)

### 1) Synthesis of Carboxylic Acid Building Blocks (ACI)

### Overview:

| ACI no. | Structure | ACI name |
|---|---|---|
| ACI-01 | 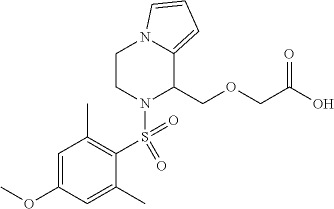 | 2-[[2-[(4-Methoxy-2,6-dimethyl- phenyl)sulfonyl]-1,2,3,4-tetrahydro- pyrrolo[1,2-a]pyrazin-1-yl]-methoxy]- acetic acid^{a} |
| ACI-02 | 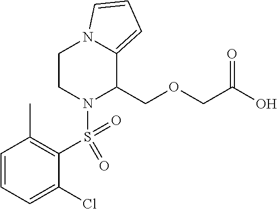 | 2-((2-(2-Chloro-6- methylphenylsulfonyl)-1,2,3,4- tetrahydropyrrolo[1,2-a]pyrazin-1- yl)methoxy)acetic acid |
| ACI-03 | 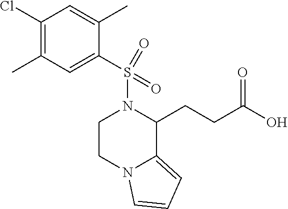 | 3-(2-(4-Chloro-2,5- dimethylphenylsulfonyl)-1,2,3,4- tetrahydropyrrolo[1,2-a]pyrazin-1- yl)propanoic acid |
| ACI-04 | 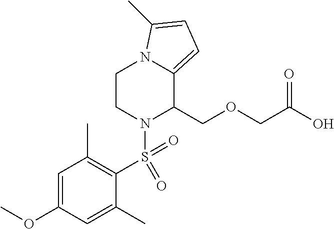 | 2-((2-(4-Methoxy-2,6- dimethylphenylsulfonyl)-6-methyl- 1,2,3,4-tetrahydropyrrolo[1,2- a]pyrazin-1-yl)methoxy)acetic acid |

^{a}Carboxylic acid ACI-01 is equivalent to carboxylic acid CC-ACI-01.

Synthesis of Carboxylic Acid ACI-01: 2-((2-(4-Methoxy-2,6-dimethylphenylsulfonyl)-1,2,3,4-tetrahydropyrrolo[1,2-a]pyrazin-1-yl)methoxy)acetic acid

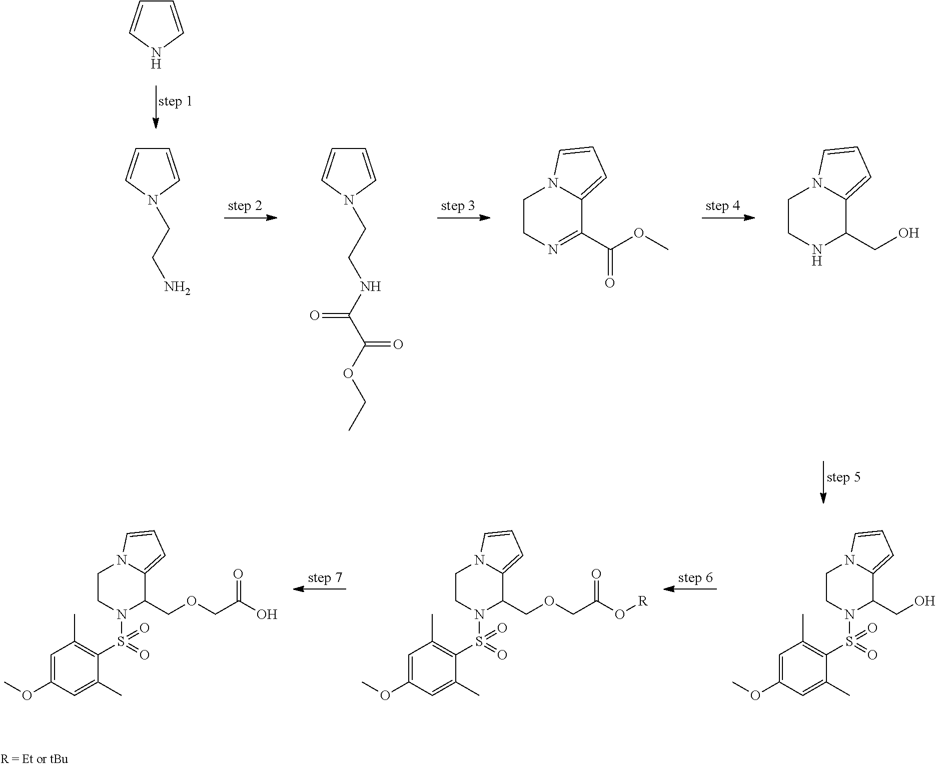

Step 1: 2-(1H-Pyrrol-1-yl)ethanamine

Sodium hydroxide (38 g, 0.95 mol, 4.0 eq.) was added to a solution of 1H-pyrrole (15 g, 0.23 mol, 1.0 eq.) in acetonitrile (250 ml) and the reaction mixture was stirred at RT for 1 h. Then 2-chloroethanamine hydrochloride (32 g, 0.27 mmol, 1.15 eq.) was added and the resulting mixture refluxed for 12 h. The reaction mixture was filtered through a bed of celite and filtrate evaporated under reduced pressure to give the crude product as a brown oil which was directly used in the next step without further purification.

Step 2: Ethyl 2-(2-(1H-pyrrol-1-yl)ethylamino)-2-oxoacetate

Method A: TEA (12.6 ml, 90.9 mmol, 2.0 eq.) was added dropwise to a solution of 2-(1H-pyrrol-1-yl)ethanamine (5 g, 45.45 mmol, 1.0 eq.) in DCM (125 ml) followed by addition of ethyloxalyl chloride (5.6 ml, 49.49 mmol, 1.1 eq.) at 0° C. The mixture was then stirred at RT for 5 h. After completion of the reaction, the solvent evaporated under reduced pressure to give the crude product as a yellow oil which was directly used in the next step.

Method B: To a solution of 2-(1H-pyrrol-1-yl)ethanamine (9.0 g, 68.18 mmol, 1.0 eq.) in DCM (250 ml) was added HOAt (9.2 g, 68.18 mmol, 1.0 eq.), EDCl (19.5 g, 102.27 mmol, 1.5 eq.) and DIPEA (29 ml, 170 mmol, 2.5 eq.). The resulting mixture was stirred at RT for 30 min. Monoethyl malonate (11.2 g, 102.27 mmol, 1.5 eq.) in DCM (50 ml) was then added to the reaction mixture. The resulting mixture was stirred at RT for 12 h. The reaction mixture was diluted with DCM (250 ml) and quenched with saturated NH₄Cl solution (150 ml). The organic layer was washed with saturated NaHCO₃ solution (100 ml) and brine (100 ml). Concentration in vacuo afforded a solid residue which was purified by column chromatography (silica gel, 40% ethyl acetate in hexanes) to obtain the desired product as a light yellow oil. Yield: 51% (7.7 g, 34.4 mmol)

Step 3: Ethyl 3,4-dihydropyrrolo[1,2-a]pyrazine-1-carboxylate

Method A: Ethyl 2-(2-(1H-pyrrol-1-yl)ethylamino)-2-oxoacetate (1 eq.) was heated with polyphosphoric acid (7 eq.) for 4 h at 90° C. and the mixture was then cooled to RT. Saturated NaHCO₃ solution was added and the mixture was extracted with DCM. The organic phase was dried (Na₂SO₄), concentrated in vacuo and purified by column chromatography (silica gel). Yield: 16%

Method B: Ethyl 2-(2-(1H-pyrrol-1-yl)ethylamino)-2-oxoacetate (7 g, 33.3 mmol, 1.0 eq.) was taken-up in a round bottom flask and cooled with an ice-bath. Pre-cooled TFA (70 ml) was slowly added to the mixture and it was then stirred at RT for 18 h. TFA was evaporated under high-vacuum and the residue then taken-up in DCM. The resulting mixture was stirred with a saturated aqueous solution of sodium bicarbonate until the pH became neutral. The organic layer was separated and the aqueous layer extracted with DCM (3×150 ml). The combined organic layers were washed with water (2×80 ml), dried over sodium sulphate and concentrated under reduced pressure to yield the crude product. This was purified by column chromatography (silica gel, 25% ethyl acetate in hexanes) to yield the pure title compound as a brown oil. Yield: 62%

Step 4: (1,2,3,4-Tetrahydropyrrolo[1,2-a]pyrazin-1-yl)methanol

A solution of ethyl 3,4-dihydropyrrolo[1,2-a]pyrazine-1-carboxylate (4 g, 20.83 mmol, 1.0 eq.) in THF (30 ml) was added dropwise to a suspension of LiAlH₄ (1.58 g, 41.66 mmol, 2.0 eq.) in THF (30 ml) at 0° C. and the reaction mixture was stirred at RT for 1 h. The reaction mixture was then quenched with saturated sodium sulphate solution (30 ml) and filtered through celite. The filtrate was evaporated under reduced pressure to give the crude product as a colorless oil which was directly used in the next step. Yield: 63% (2 g, 13.16 mmol)

Step 5: (2-(4-Methoxy-2,6-dimethylphenylsulfonyl)-1,2,3,4-tetrahydropyrrolo[1,2-a]pyrazin-1-yl)methanol

A solution of 4-methoxy-2,6-dimethylbenzene-1-sulfonyl chloride (9.0 g, 38.46 mmol, 1.0 eq.) in DCM (30 ml) is added dropwise to a solution of (1,2,3,4-tetrahydropyrrolo[1,2-a]pyrazin-1-yl)methanol (5.26 g, 34.61.46 mmol, 1.1 eq.) and TEA (2 eq.) in DCM (100 ml) at 0° C. The reaction mixture was stirred at RT for 1 h. The mixture was then diluted with DCM and washed with water (100 ml) and brine (50 ml). The organics were dried over sodium sulphate and concentrated under reduced pressure to give the crude product which was purified by column chromatography (silica gel, 100% DCM) to yield the desired product as an off-white solid. Yield: 49% (6.7 g, 19.14 mmol)

Step 6: tert-Butyl 2-((2-(4-methoxy-2,6-dimethylphenylsulfonyl)-1,2,3,4-tetrahydropyrrolo[1,2-a]pyrazin-1-yl)methoxy)acetate and Ethyl 2-((2-(4-methoxy-2,6-dimethylphenylsulfonyl)-1,2,3,4-tetrahydropyrrolo[1,2-a]pyrazin-1-yl)methoxy)acetate

Method A (R=tBu): t-Butyl bromoacetate (4.5 eq.) and 35% aqueous KOH solution were added to a solution of (2-(4-methoxy-2,6-dimethylphenylsulfonyl)-1,2,3,4-tetrahydropyrrolo[1,2-a]pyrazin-1-yl)methanol (1 eq.) and TBACl (1 eq.) in toluene (1.5 ml) and the mixture was stirred overnight at RT. The phases were separated and the organic phase was washed with water, dried (Na₂SO₄) and concentrated in vacuo. The crude product was used in the following step without further purification.

Method B (R=Et): To a cooled (0° C.) suspension of NaH (5.48 g, 22.84 mmol, 2.0 eq.) in THF (50 ml) was added dropwise a solution of (2-(4-methoxy-2,6-dimethylphenylsulfonyl)-1,2,3,4-tetrahydropyrrolo[1,2-a]pyrazin-1-yl)methanol (4 g, 11.42 mmol, 1.0 eq.) in THF (50 ml) and the reaction mixture was stirred at RT for 1 h. Then a solution of ethylbromoacetate (1.52 ml, 13.70 mmol, 1.2 eq.) in THF (50 ml) was added dropwise to the reaction mixture at 0° C. and it was stirred at RT for 2 h. The reaction mixture was quenched with ammonium chloride solution (50 ml) and filtered through a bed of celite. The filtrate was concentrated under reduced pressure to give the crude product which was purified by column chromatography (silica gel, 15% ethyl acetate in hexanes) to yield the desired product as a white solid. Yield: 64% (3.2 g, 7.34 mmol)

Step 7: 2-((2-(4-Methoxy-2,6-dimethylphenylsulfonyl)-1,2,3,4-tetrahydropyrrolo[1,2-a]pyrazin-1-yl)methoxy)acetic acid

Method A (R=tBu): KOH (2 eq.) was added to a solution of tert-butyl 2-((2-(4-methoxy-2,6-dimethylphenylsulfonyl)-1,2,3,4-tetrahydropyrrolo[1,2-a]pyrazin-1-yl)methoxy)acetate (1 eq.) in MeOH at RT and the mixture was stirred for 3 h. The reaction mixture was concentrated in vacuo and the residue taken-up in water and washed with ethyl acetate. The aqueous phase was acidified to pH 3 to 4 and then extracted with ethyl acetate. The organic phase was extracted with water and saturated sodium chloride solution, dried (Na₂SO₄), concentrated in vacuo and used in the following step without further purification.

Method B (R=Et): Ethyl-2-((2-(4-methoxy-2,6-dimethylphenylsulfonyl)-1,2,3,4-tetrahydropyrrolo[1,2-a]pyrazin-1-yl)methoxy)acetate (4.58 mmol, 1 eq.) was dissolved in ethanol (27 ml) and water (6 ml) and potassium hydroxide (1 M in water, 2 eq.) was added. The resulting mixture was stirred at RT for 16 h. Ethanol was removed in vacuo and the residue was taken up in water and diethylether (20 ml each). The aqueous phase was adjusted to pH 3 with 1 M HCl (aq) and extracted with ethyl acetate (3×30 ml). The combined organic layers were dried over MgSO₄ and concentrated in vacuo to yield the desired compound. Yield: 1.89 g (>99%)

Synthesis of carboxylic acid ACI-02: 2-((2-(2-Chloro-6-methylphenylsulfonyl)-1,2,3,4-tetrahydropyrrolo[1,2-a]pyrazin-1-yl)methoxy)acetic acid

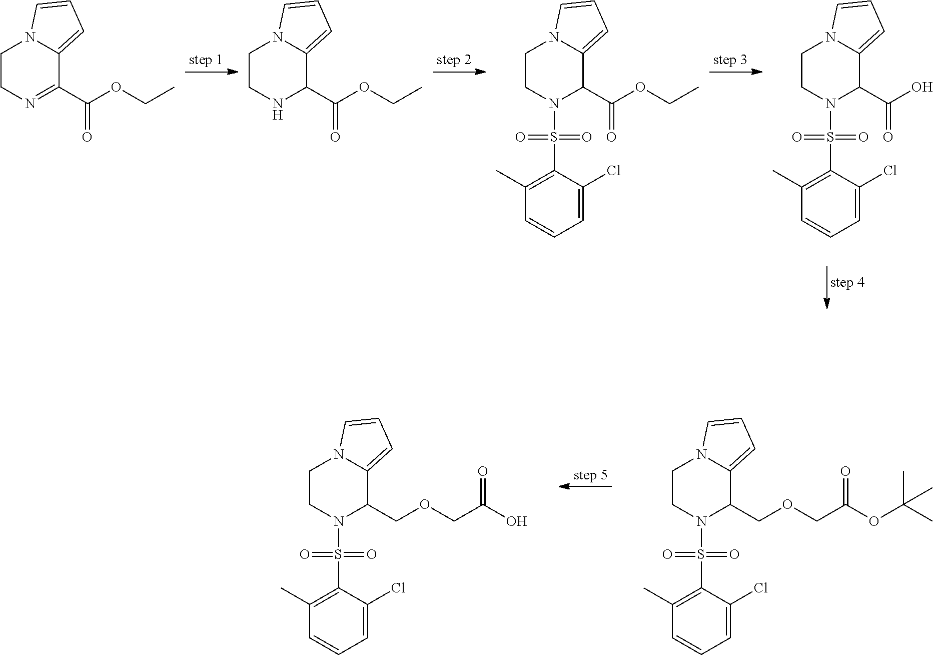

Step 1: Ethyl 1,2,3,4-tetrahydropyrrolo[1,2-a]pyrazine-1-carboxylate

A mixture of methanol (45 ml) and water (5 ml) was cooled to 0° C. and added to ethyl 3,4-dihydropyrrolo[1,2-a]pyrazine-1-carboxylate (2.35 g, 12.23 mmol) (see step 2 product of ACI-1). NaBH₄ (555 mg, 14.67 mmol) was then added in portions over 10 min. The reaction solution was stirred for 1 h at 0° C. and then the bulk of the organic solvent was removed under reduced pressure. NH₃ solution (10%, 200 ml) and DCM (200 ml) were added followed by saturated NaCl solution (150 ml) to improve phase separation. The phases were separated and the aqueous phase was extracted twice with DCM (200 ml each). The combined organic phases were dried over Na₂SO₄ and concentrated in vacuo. The crude product obtained was used in the next step on the same day.

Step 2: Ethyl 2-(2-chloro-6-methylphenylsulfonyl)-1,2,3,4-tetrahydropyrrolo[1,2-a]pyrazine-1-carboxylate

The reaction was performed under an N₂ atmosphere. Ethyl 1,2,3,4-tetrahydropyrrolo[1,2-a]pyrazine-1-carboxylate (0.84 g, 4.32 mmol) was dissolved in DCM (10 ml), TEA (1.22 ml, 8.65 mmol) was added and the reaction mixture was cooled to 0° C. 2-Chloro-6-methylbenzene-1-sulfonyl chloride (5.19 mmol) was added and the mixture was stirred for 1 h at 0° C. and then overnight at RT. The solvent was removed under reduced pressure and purification was performed by column chromatography (silica gel, heptane/ethyl acetate, 3:1).

Step 3: (2-(2-Chloro-6-methylphenylsulfonyl)-1,2,3,4-tetrahydropyrrolo[1,2-a]pyrazin-1-yl)methanol

The reaction was performed under an N₂ atmosphere. LIBH₄ solution (2 M in THF, 2 ml, 4 mmol) was added to a solution of ethyl 2-(2-chloro-6-methylphenylsulfonyl)-1,2,3,4-tetrahydropyrrolo[1,2-a]pyrazine-1-carboxylate (0.82 g, 2.038 mmol) in dry THF (50 ml) and the mixture was stirred overnight at RT. Na₂SO₄.10H₂O was added until no further gas evolution was observed. The reaction mixture was stirred for 1 d at RT and then filtered over Na₂SO₄. The residue was washed with THF (approx. 50 ml) and the combined organic phases were concentrated in vacuo. The crude product obtained used in the next step without further purification.

Step 4: tert-Butyl 2-((2-(2-chloro-6-methylphenylsulfonyl)-1,2,3,4-tetrahydropyrrolo[1,2-a]pyrazin-1-yl)methoxy)acetate

Aqueous NaOH solution (35%, 50 ml) followed by tert-butyl bromoacetate (1.62 ml, 11.11 mmol) were added to a solution of (2-(2-chloro-6-methylphenylsulfonyl)-1,2,3,4-tetrahydropyrrolo[1,2-a]pyrazin-1-yl)methanol (0.79 g, max. 2.01 mmol) and n-Bu₄NCl (0.122 g, 0.439 mmol) in DCM (50 ml). The reaction mixture was stirred for 3 h at RT and was then diluted with DCM (50 ml). The organic phase was washed with water (3×50 ml) and then with saturated NaCl solution (50 ml). The organic phase was dried (Na₂SO₄), concentrated in vacuo and purified by column chromatography (silica gel, heptane/ethyl acetate, 4:1).

Step 5: 2-((2-(2-Chloro-6-methylphenylsulfonyl)-1,2,3,4-tetrahydropyrrolo[1,2-a]pyrazin-1-yl)methoxy)acetic acid

tert-Butyl 2-((2-(2-chloro-6-methylphenylsulfonyl)-1,2,3,4-tetrahydropyrrolo[1,2-a]pyrazin-1-yl)methoxy)acetate (440 mg, 0.93 mmol) was dissolved in methanol (9 ml), THF (4 ml) and water (1 ml). NaOH (371 mg, 9.28 mmol) was added and then the mixture was stirred for 2 h at RT. The bulk of the organic solvent was then removed in vacuo. Ice (100 ml) and DCM (50 ml) were added and then aqueous KHSO₄ solution (0.5 M, 50 ml) was added to the mixture. The phases were separated and the aqueous phase was extracted once more with DCM. The combined organic phases were dried over Na₂SO₄ and concentrated in vacuo. The crude product obtained was used in the next step without further purification.

Synthesis of carboxylic acid ACI-03: 3-(2-(4-Chloro-2,5-dimethylphenylsulfonyl)-1,2,3,4-tetrahydropyrrolo[1,2-a]pyrazin-1-yl)propanoic acid

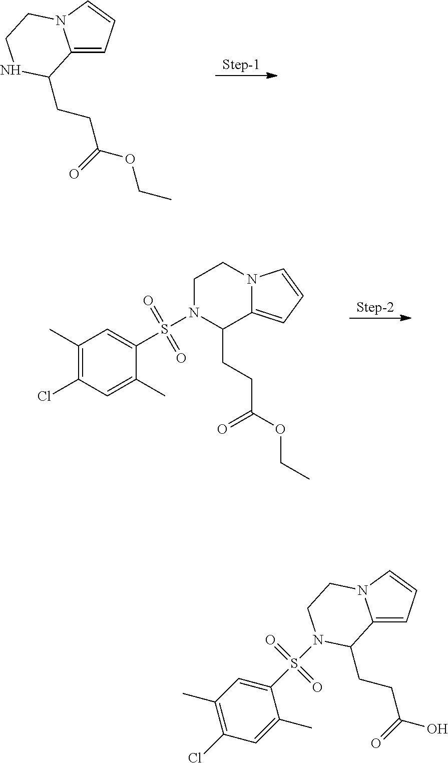

Step 1: Ethyl 3-(2-(4-chloro-2,5-dimethylphenylsulfonyl)-1,2,3,4-tetrahydropyrrolo[1,2-a]pyrazin-1-yl)propanoate

To a stirred solution of ethyl 3-(1,2,3,4-tetrahydropyrrolo[1,2-a]pyrazin-1-yl)propanoate (2.0 g, 9.01 mmol, 1.0 eq.) (for synthesis details see parallel synthesis section CC_ACI-05) in DCM (5 ml) was added triethylamine (2.49 ml, 18.018 mmol, 2.0 eq.). The reaction mixture was then cooled to 0° C. (and optionally stirred for approximately 10 min). 4-Chloro-2,5-dimethylbenzene-1-sulfonyl chloride (2.58 g, 10.8108 mmol, 1.2 eq.) was added and the reaction mixture then stirred for 4 h at RT. The mixture was diluted with DCM (100 ml) and washed with water (50 ml) and brine (50 ml). The organic layer is dried over Na₂SO₄ and concentrated in vacuo to yield the crude material, which is purified by silica gel column chromatography (silica gel; 25% ethyl acetate/hexanes) to obtain the desired product as a light yellow oil. Yield: 52% (1.1 g, 2.5943 mmol)

Step 2: 3-(2-(4-Chloro-2,5-dimethylphenylsulfonyl)-1,2,3,4-tetrahydropyrrolo[1,2-a]pyrazin-1-yl)propanoic acid

To a cooled (0° C.) stirred solution of ethyl 3-(2-(4-chloro-2,5-dimethylphenylsulfonyl)-1,2,3,4-tetrahydropyrrolo[1,2-a]pyrazin-1-yl)propanoate (500 mg, 1.179 mmol, 1.0 eq.) in a mixture of MeOH:THF (1:1, 10 ml) was added dropwise lithium hydroxide monohydrate (99 mg, 2.358 mmol, 2.0 eq.) in water (5 ml). The reaction mixture was stirred at room temperature for approximately 2 h. The mixture was concentrated in vacuo and the residue obtained dissolved in water (50 ml). This aqueous layer was acidified to pH≈3 using 1 N HCl. The aqueous layer was extracted with DCM (2×80 ml). The combined organic layers were washed with brine (40 ml), dried over Na₂SO₄ and concentrated in vacuo to yield the desired product as a white solid which is used for the next step without further purifications. Yield: 64% (300 mg, 0.7575 mmol)

Alternatively ethyl 3-(1,2,3,4-tetrahydropyrrolo[1,2-a]pyrazin-1-yl)propanoate can be synthesised as follows:

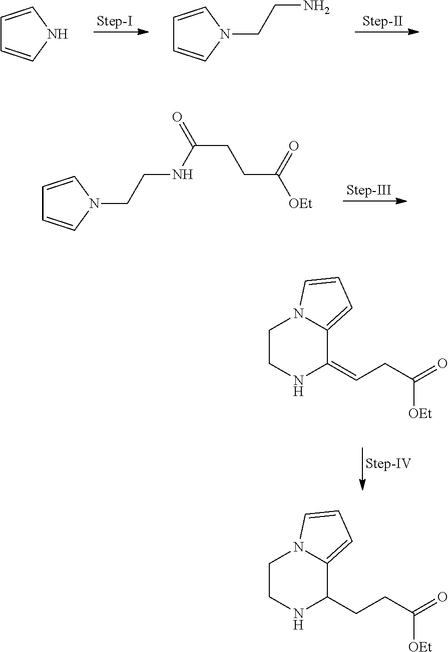

Step I: 2-(1H-Pyrrol-1-yl)ethanamine

Sodium hydroxide anhydrous (71.6 g, 1.79 mol, 4.0 eq.) was added to a solution of 1H-pyrrole (30.0 g, 0.447 mol, 1.0 eq.) in acetonitrile (500 ml) at 0° C. and the mixture was stirred at RT for 1 h. 2-Chloro ethylamine hydrochloride (59.72 g, 0.514 mol, 1.15 eq.) was added and the reaction mixture was heated to reflux for 12 h. The reaction mixture was then cooled to RT, filtered through celite and the filter cake washed with DCM (200 ml). The filtrate was concentrated under reduced pressure to give the crude product which was purified by vacuum distillation to afford the desired product as a colorless oil. Yield: 50% (25.0 g, 0.227 mol)

Step II: Ethyl 4-(2-(1H-pyrrol-1-yl)ethylamino)-4-oxobutanoate

Ethyl succinylchloride (11.4 ml, 79.99 mmol, 1.1 eq.) was added dropwise to a solution of 2-(1H-pyrrol-1-yl)ethanamine (8.0 g, 72.7272 mmol, 1.0 eq.) and TEA (20 ml, 145.45 mmol, 2.0 eq.) in DCM (80 ml) at 0° C. The resulting reaction mixture was stirred at RT for 5 h. After completion of the reaction (monitored by TLC), the solvent was evaporated under reduced pressure to obtain the crude product which was purified by column chromatography (silica gel; 25% ethyl acetate/hexane) to yield the desired product as a yellow oil. Yield: 46% (8.0 g, 33.6134 mmol)

Step III: (E)-Ethyl 3-(3,4-dihydropyrrolo[1,2-a]pyrazin-1(2H)-ylidene)propanoate

TFA (36 ml) was added to ethyl 4-(2-(1H-pyrrol-1-yl)ethylamino)-4-oxobutanoate (8.0 g, 33.6134 mmol, 1.0 eq.) at 0° C. and the resulting mixture was stirred at RT for 16 h. The reaction mixture was then concentrated and the residue diluted with water (200 ml). The mixture was neutralized to pH≈7 with NaHCO₃ and extracted with DCM (3×200 ml). The combined organic layers were dried over Na₂SO₄ and concentrated in vacuo to give the crude product, which was used for the next step without further purification. Yield: 8.0 g (crude)

Step IV: Ethyl 3-(1,2,3,4-tetrahydropyrrolo[1,2-a]pyrazin-1-yl)propanoate

A solution of (E)-ethyl 3-(3,4-dihydropyrrolo[1,2-a]pyrazin-1(2H)-ylidene)propanoate (400 mg, 1.8181 mmol, 1.0 eq.) in MeOH (4 ml) was degassed with Argon followed by addition of Pd—C (100 mg). The reaction mixture was stirred under H₂-balloon pressure at RT for 6 h. After completion of the reaction, the catalyst was filtered off and the filtrate was concentrated to give the desired product as a light brown sticky liquid which was used for the next step without further purification. Yield: 250 mg (crude)

Synthesis of carboxylic acid ACI-04: 2-((2-(4-Methoxy-2,6-dimethylphenylsulfonyl)-6-methyl-1,2,3,4-tetrahydropyrrolo[1,2-a]pyrazin-1-yl)methoxy)acetic acid

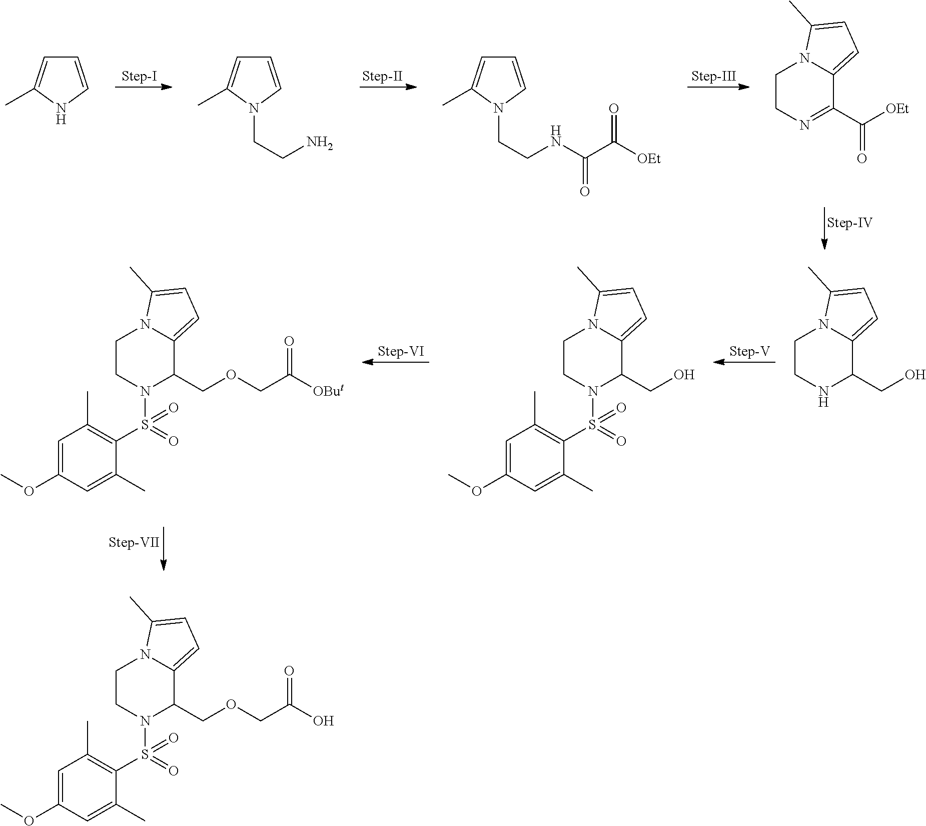

Step I: 2-(2-Methyl-1H-pyrrol-1-yl)ethanamine

Sodium hydroxide anhydrous (9.87 g, 0.246 mol, 4.0 eq.) and TBAHS (0.838 g, 0.002 mol, 0.04 eq.) were added to a solution of 2-methyl-1H-pyrrole (5.0 g, 0.061 mol, 1.0 eq.) in acetonitrile (180 ml) at 0° C. and the mixture was stirred at RT for 1 h. 2-Chloroethylamine hydrochloride (8.59 g, 0.074 mol, 1.2 eq.) was added to the reaction mixture and it was then heated at reflux for 16 h. The reaction mixture was cooled to RT, filtered through celite and washed with 10% MeOH/DCM (200 ml). The filtrate was concentrated under reduced pressure to give the crude product which was used in the next step without further purification. Yield: 100%, crude (7.6 g, 0.061 mol)

Step II: Ethyl 2-(2-(2-methyl-1H-pyrrol-1-yl)ethylamino)-2-oxoacetate

Ethyl oxalylchloride (8.23 ml, 0.073 mol, 1.2 eq.) was added dropwise to a solution of 2-(2-methyl-1H-pyrrol-1-yl)ethanamine (7.6 g, 0.061 mol, 1.0 eq.) and TEA (21.25 ml, 0.153 mol, 2.5 eq.) in DCM (200 ml) at 0° C. The resulting reaction mixture was stirred at RT for 5 h. After completion of the reaction (monitored by TLC), the reaction mixture was diluted with DCM (100 ml), washed with water (200 ml) and brine (200 ml), and dried over sodium sulfate. The solvent was evaporated under reduced pressure to yield the crude product which was purified by column chromatography (silica gel; 30% ethyl acetate/hexanes) to afford the desired compound as a pale yellow gummy material. Yield: 27% (3.8 g, 16.964 mmol)

Step III: Ethyl 6-methyl-3,4-dihydropyrrolo[1,2-a]pyrazine-1-carboxylate

TFA (38 ml) was added to ethyl 2-(2-(2-methyl-1H-pyrrol-1-yl)ethylamino)-2-oxoacetate (3.8 g, 16.964 mmol, 1.0 eq.) at 0° C. and the mixture was stirred at RT for 16 h. The reaction mixture was then concentrated and diluted with water (200 ml). The mixture was basified to pH˜9.0 with NaHCO₃ and extracted with DCM (3×200 ml). The combined organic layers were dried over Na₂SO₄ and concentrated in vacuo. The crude product was purified by column chromatography (silica gel; 100% ethyl acetate) to yield the desired product as a brown solid. Yield: 86% (3.0 g, 14.563 mmol)

Step IV: (6-Methyl-1,2,3,4-tetrahydropyrrolo[1,2-a]pyrazin-1-yl)methanol

A solution of ethyl 6-methyl-3,4-dihydropyrrolo[1,2-a]pyrazine-1-carboxylate (2.4 g, 11.650 mmol, 1.0 eq.) in THF (15 ml) was added dropwise to a suspension of LAH (885 mg, 23.300 mmol, 2.0 eq.) in THF (40 ml) at 0° C. The resulting mixture was stirred at RT for 2 h. The reaction mixture was then quenched with water:THF (1:9, 10 ml), followed by addition of 10% NaOH solution (2 ml). The mixture was diluted with THF (50 ml) and stirred at RT for 1 h, before being filtered through celite. The filtrate was concentrated under reduced pressure to yield the desired product as a red solid which was used in the next step without further purification. Yield: 93% (1.8 g, 26.31 mmol)

Step V: (2-(4-Methoxy-2,6-dimethylphenylsulfonyl)-6-methyl-1,2,3,4-tetrahydropyrrolo[1,2-a]pyrazin-1-yl)methanol

A solution of 4-methoxy-2,6-dimethyl benzenesulfonyl chloride (3.04 g, 13.012 mmol, 1.2 eq.) in DCM (10 ml) was added to a solution of (6-methyl-1,2,3,4-tetrahydropyrrolo[1,2-a]pyrazin-1-yl)methanol (1.8 g, 10.843 mmol, 1.0 eq.) and TEA (3.76 ml, 27.108 mmol, 2.5 eq.) in DCM (20 ml) at 0° C. and the reaction mixture was stirred at RT for 5 h. The mixture was then diluted with DCM (100 ml), washed with water (50 ml) and brine (50 ml), and dried over sodium sulfate. The solvent was evaporated under reduced pressure to give the crude product which was purified by column chromatography (silica gel; 30% ethyl acetate/hexanes) to afford the desired product as a light brown sticky solid. Yield: 51% (2.0 g, 5.494 mmol)

Step VI: tert-Butyl 2-((2-(4-methoxy-2,6-dimethylphenylsulfonyl)-6-methyl-1,2,3,4-tetrahydropyrrolo[1,2-a]pyrazin-1-yl)methoxy)acetate

30% NaOH solution (7 ml) was added to a solution of (2-(4-methoxy-2,6-dimethylphenylsulfonyl)-6-methyl-1,2,3,4-tetrahydropyrrolo[1,2-a]pyrazin-1-yl)methanol (800 mg, 2.197 mmol, 1.0 eq.) in DCM (14 ml) followed by the addition of TBAB (707 mg, 2.197 mmol, 1.0 eq.) and tert-butyl bromoacetate (0.487 ml, 3.296 mmol, 1.5 eq.) at 0° C. The reaction mixture was stirred at RT for 48 h. The mixture was diluted with DCM (100 ml) and washed with water (50 ml) and brine (50 ml). The organic layer was dried over sodium sulfate and evaporated under reduced pressure to give the crude product which was purified by column chromatography (silica gel; 30% ethyl acetate/hexanes) to yield the desired product as an off-white sticky solid. Yield: 95% (1.0 g, 2.092 mmol)

Step VII: 2-((2-(4-Methoxy-2,6-dimethylphenylsulfonyl)-6-methyl-1,2,3,4-tetrahydropyrrolo[1,2-a]pyrazin-1-yl)methoxy)acetic acid

Solid KOH (175 mg, 3.138 mmol, 3.0 eq.) was added to a solution of tert-butyl 2-((2-(4-methoxy-2,6-dimethylphenylsulfonyl)-6-methyl-1,2,3,4-tetrahydropyrrolo[1,2-a]pyrazin-1-yl)methoxy)acetate (500 mg, 1.046 mmol, 1.0 eq.) in MeOH (10 ml) at 0° C. and the resulting mixture was stirred at RT for 16 h. The reaction mixture was concentrated, the residue dissolved in water (30 ml) and washed with ethyl acetate (50 ml). The aqueous layer was acidified to pH≈3-4 with 1N HCl solution and extracted with DCM (2×50 ml). The combined organic layers were washed with water (50 ml) and brine (50 ml), and were then dried over Na₂SO₄. The solvent was evaporated under reduced pressure to yield the desired product which was used in the next step without further purification. Yield: 90% (400 mg, 0.947 mmol)

### 2) Synthesis of Amine Building Blocks (AMN)

### Overview:

| AMN no. | Structure | AMN name |
|---|---|---|
| AMN-01 | 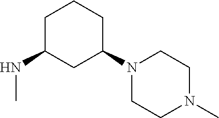 | (1S,3R)-N-methyl-3-(4-methylpiperazin- 1-yl)cyclohexanamine^{a} |
| AMN-03 | 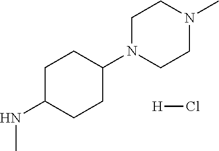 | N-Methyl-4-(4-methylpiperazin-1- yl)cyclohexanamine hydrochloride |

^{a}Amine AMN-01 was synthesised in analogy to the method described in WO2010/017850.

Synthesis of amine AMN-01: (1S,3R)-N-methyl-3-(4-methylpiperazin-1-yl)cyclohexanamine

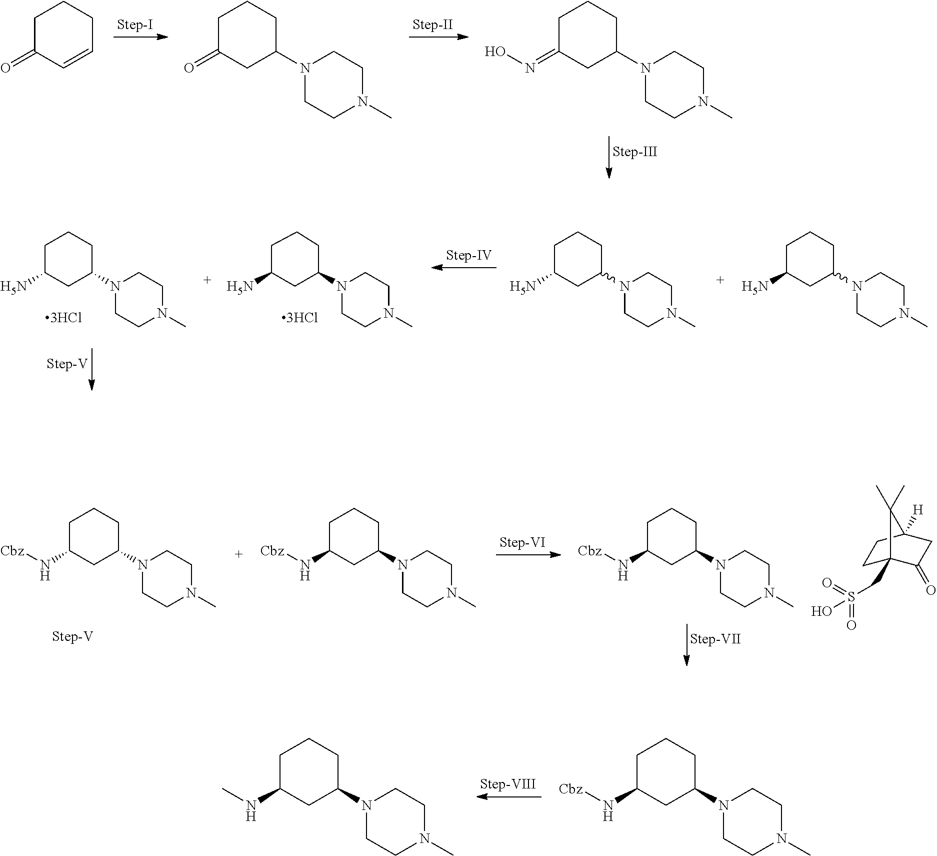

Steps 1 & 2: 3-(4-Methylpiperazin-1-yl)cyclohexanone oxime

Cyclohex-2-enone (1 eq.) and N-methyl-piperazine (1 eq.) are stirred in ethanol for approx. 1 h at RT. Subsequently the mixture is diluted with ethanol and cooled to 0° C. Potassium carbonate (1.13 eq.) and hydroxylamine hydrochloride (1.13 eq.) are added portionwise to the reaction mixture. Then the mixture is stirred at 0° C. for approximately 30 min, followed by additional approximately 30 min at RT. The suspension is filtered, diluted with ethanol and then concentrated. The residue is diluted with tetrahydrofuran, filtered and again diluted with tetrahydrofuran. Then the solvent is, at least in part, removed and to the residue is added n-heptane. After some time, upon cooling the product crystallises from the solution and is collected by filtration, washed with n-heptane and dried to give the title compound.

or

To the stirred solution of 2-cyclohexen-1-one (100.0 g, 1041.6 mmol 1.0 eq.) in ethanol (300 ml) was added dropwise a solution of N-methylpiperazine (104.16 g, 1041.6 mmol, 1.0 eq.) and the resulting reaction mixture was stirred at RT for 5 h. The reaction mixture was diluted with 600 ml of ethanol and cooled to 0° C. K₂CO₃ (172.48 g, 1249.9 mmol, 1.2 eq.) was added followed by portionwise the addition of NH₂OH.HCl (86.7 g, 1249.9 mmol, 1.2 eq.). Stirring was continued for 30 min at 0° C., followed by 16 h at RT. The reaction mixture was filtered through a bed of celite and washed with ethanol (500 ml). The filtrate was concentrated under reduced pressure. The concentrated mass was stirred with THF (100 ml) and n-hexane (500 ml) for 3 h and then filtered to yield the title compound as a white solid which was used in the next step without further purification. Yield: 54% (120.0 g, 568.7 mmol)

Steps 3 & 4: (cis)-3-(4-Methylpiperazin-1-yl)cyclohexanamine trihydrochloride

To a mixture of 3-(4-Methylpiperazin-1-yl)cyclohexanone oxime (1 eq.), toluene, ethanol and ethanolic ammonia is added Raney nickel and the mixture is hydrogenated under a hydrogen atmosphere (approximately 5 bar) at 30° C. Subsequently the mixture is filtered and diluted with ethanol and methanol, before the solvent is removed in vacuo. After addition of methanol to the residue the solvent is again removed in vacuo. The residue is diluted with methanol, warmed to 50° C. and ethanolic HCl (10 M) is added. Upon cooling to RT the desired product precipitates and is collected by filtration. After an optional recrystallisation from methanol the product is dried.

or

Step 3: To a stirred suspension of LAH (13.15 g, 355.45 mmol, 2.5 eq.) in THF (600 ml) was added portionwise 3-(4-methylpiperazin-1-yl)cyclohexanone oxime (30.0 g, 142.18 mmol, 1.0 eq.) at 0° C. After complete addition, the reaction mixture was warmed to RT and then refluxed for 14 h. The reaction mixture was cooled to 0° C. and quenched with 15% NaOH solution (15 ml). The mixture was then filtered and washed with 10% MeOH/DCM (500 ml). The filtrate was concentrated under reduced pressure to afford crude product as a sticky solid. Yield: 67% (19.0 g, 96.44 mmol)

Step 4: To the stirred solution of the product obtained from step (i) (59.0 g, 284.26 mmol) in methanol (70 ml) was added dropwise ethanolic/HCl (7N, 150 ml) at 50° C. and the reaction mixture was stirred at the same temperature for 3 h. The reaction mixture was cooled to RT and the precipitate separated by filtration. The solid residue was recrystallized from methanol to give the desired product. Yield: 32% (28.0 g, 91.80 mmol)

Steps 5 & 6: Benzyl (1S,3R)-3-(4-methylpiperazin-1-yl)cyclohexylcarbamate ((1S,4R)-7,7-dimethyl-2-oxobicyclo[2.2.1]heptan-1-yl)methanesulfonate

Potassium carbonate (4.25 eq.) is dissolved in water and (cis)-3-(4-methylpiperazin-1-yl)cyclohexanamine trihydrochloride (1 eq.) is added. Then a solution of benzyloxycarbonyloxysuccinimide (1 eq.) in toluene is added at RT. The mixture is stirred for 30 min at RT and then water is added and stirring continued for 5 min. The phases are separated and the organic phase is concentrated. Isopropyl acetate is added to the residue obtained at 65° C. The solution is cooled to RT and added to a mixture of (1S)(+) camphorsulfonic acid (0.5 eq.) and water. The resulting mixture is heated to reflux until a clear solution is obtained. The solution is slowly cooled to RT and the resulting suspension stirred for approximately 3 h. The precipitate is collected by filtration and washed with isopropyl acetate (2×). After an optional recrystallization from isopropyl acetate/ethanol the product is dried.

### Specifically:

Step 5: To a mixture of (cis)-3-(4-methylpiperazin-1-yl)cyclohexanamine trihydrochloride (5.0 g, 16.39 mmol, 1.0 eq.) and K₂CO₃ (9.05 g, 65.56 mmol, 4.0 eq.) in water (15 ml) was added a solution of Cbz-Cl (2.85 g, 16.39 mmol, 1.0 eq.) in toluene (25 ml) at 0° C. and then the reaction mixture was stirred at RT for 16 h. The mixture was diluted with water (15 ml) and the layers were separated. The organic layer was dried over sodium sulfate and concentrated to yield the crude product which was purified by column chromatography (neutral alumina; 1% MeOH/DCM). Yield: 37% (2.0 g, 6.04 mmol)

Step 6: A mixture of the product from step (i) (2.0 g, 6.04 mmol, 1.0 eq.) in toluene (2.5 ml) and isopropyl acetate (12 ml) was heated at 65° C. for 15 min and then the reaction mixture was cooled to RT. (1S)-(+)Camphorsulfonic acid (700 mg, 3.02 mmol, 0.5 eq.) and water (0.1 ml) were added to the reaction mixture and it was heated at 75° C. for 3 h. The reaction mixture was cooled to RT and stirred for 3 h. The mixture was kept at RT overnight and the solid product was separated by filtration. It was washed with isopropyl acetate (20 ml) to give 1.0 g of crude product which was re-crystallized from EtOH and isopropyl acetate to afford desired product as a white solid. Yield: 14% (500 mg, 0.888 mmol)

Steps 7 & 8: (1S,3R)-N-Methyl-3-(4-methylpiperazin-1-yl)cyclohexanamine

Benzyl (1S,3R)-3-(4-methylpiperazin-1-yl)cyclohexylcarbamate ((1S,4R)-7,7-dimethyl-2-oxobicyclo[2.2.1]heptan-1-yl)methanesulfonate (1 eq.) is suspended in water and toluene and NaOH solution (50%) (1.05 eq.) is added. The mixture is stirred for approximately 5 min at RT and then the aqueous phase is separated. The organic phase is concentrated in part and the residue obtained is dissolved in toluene and THF. The resulting solution is added to a mixture of LAH (10% in THF) (1.45 eq.), THF and toluene over 30 min at 85° C. The reaction mixture is then cooled to approximately 35° C. and a mixture of water and THF is added. This is followed by the addition of aq. NaOH and water. The resulting suspension is filtered and toluene is added. The solvent is removed in vacuo and to the residue is added methanol. To this mixture is added ethanolic HCl (10 M) at RT, followed by the addition of toluene. The solid is collected by filtration, washed with toluene/methanol (2×) and dried to afford the desired product. Specifically:

Step 7: A solution of NaOH (0.234 g, 5.852 mmol, 1.1 eq.) in water (15 ml) was added to a suspension of benzyl (1S,3R)-3-(4-methylpiperazin-1-yl)cyclohexylcarbamate ((1S,4R)-7,7-dimethyl-2-oxobicyclo[2.2.1]heptan-1-yl)methanesulfonate (3.0 g, 5.32 mmol, 1.0 eq.) in toluene (15 ml) and the resulting mixture was stirred at RT for 30 min. The reaction mixture was then diluted with toluene (15 ml) and the organic layer was separated. The organic layer was washed with brine solution (15 ml) and dried over sodium sulfate. The solvent was evaporated under reduced pressure to give the desired product. Yield: 96% (1.7 g, 5.13 mmol)

Step 8: A solution of the product from step (i) (2.0 g, 6.042 mmol, 1.0 eq.) in THF (10 ml) was added dropwise to a suspension of LAH (0.345 g, 9.06 mmol, 1.5 eq.) in THF (30 ml) at 0° C. After complete addition the reaction mixture was warmed to RT and then refluxed for 30 min. The mixture was then cooled to 0° C. and quenched with 10% NaOH solution (0.35 ml). It was filtered and washed with THF (2×50 ml). The filtrate was concentrated under reduced pressure to afford the desired product as a colorless sticky liquid. Yield: 94% (1.2 g, 5.68 mmol)

Synthesis of amine AMN-03: N-Methyl-4-(4-methylpiperazin-1-yl)cyclohexanamine hydrochloride

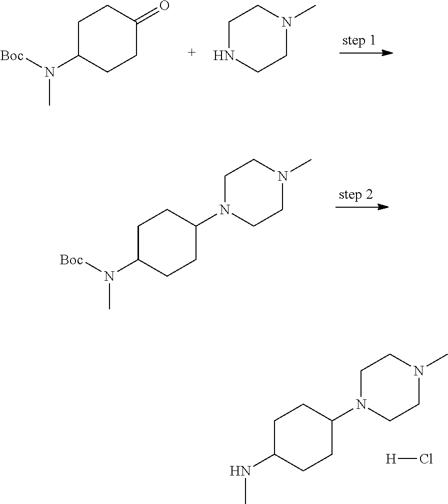

Step 1: tert-Butyl methyl(4-(4-methylpiperazin-1-yl)cyclohexyl)carbamate

To a solution of tert-butyl methyl(4-oxocyclohexyl)carbamate (0.45 g, 1.98 mmol, 1 eq.), 1-methylpiperazine (0.25 ml, 2.257 mmol, 1.14 eq.) and acetic acid (0.3 ml) in dichloromethane (19 ml) was added sodium triacetoxyborohydride (0.58 g, 2.772 mmol, 1.4 eq.) and the reaction mixture was stirred at RT for 16 h. The reaction mixture was diluted with sodium bicarbonate solution and extracted with dichloromethane (2×50 ml). The organic layer was washed with brine, dried over sodium sulfate and concentrated under reduced pressure. The crude product was purified by column chromatography (silica gel, ethyl acetate/EtOH). Yield: 76%

Step 2: N-Methyl-4-(4-methylpiperazin-1-yl)cyclohexanamine hydrochloride

To a solution of the product from step 1 (0.46 g, 1.477 mmol, 1.0 eq.) in ethanol (5 ml) was added acetyl chloride (0.52 ml, 7.385 mmol, 5.0 eq.) at 0° C. and the mixture was stirred for 16 h at RT. The reaction mixture was concentrated under reduced pressure and dried under vacuum to yield the crude product, which was employed in the next step without further purification. Yield: >100%

### 3) Single Compounds (SC)

### General Method for Synthesis of Single Compounds (SC)

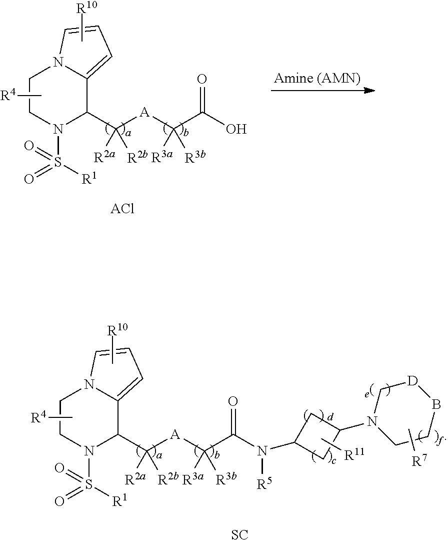

### General Procedure 1 (GWI-1)

To a cooled (0° C.) solution/suspension of the carboxylic acid (ACI) (1.0 eq.) in THF (3-4 ml) were added HATU (1.0-1.5 eq.) and DIPEA (2.0-4.0 eq.) and the reaction mixture was allowed to stir for 15 to 30 min. A solution of the amine (AMN) (1.2 eq.) in THF (3-4 ml) was added to the reaction mixture and it was stirred at RT for 16 h. The reaction mixture was diluted with ethyl acetate or DCM and successively washed with sat. NaHCO₃ solution, sat. NH₄Cl solution, water and brine, and dried over sodium sulfate. The solvent is evaporated under reduced pressure to give the crude which is purified by column chromatography (silica gel, MeOH/DCM) to yield the desired compound.

### General Procedure 2 (GWI-2)

To a cooled (0° C.) solution/suspension of the carboxylic acid (ACI) (1 eq.) in DCM were added EDCl (1.2-1.5 eq.), HOAt or HOBt (0.2-1.0 eq.) and DIPEA (2.0-4.0 eq.) and the reaction mixture was allowed to stir for 15 min. The amine (AMN) (1.0-1.1 eq.) was added and the reaction mixture was stirred at RT for 12-48 h. The reaction mixture was diluted with DCM or ethyl acetate and successively washed with sodium bicarbonate solution, ammonium chloride solution, water and brine, and dried over sodium sulfate. The solvent was evaporated under reduced pressure to give the crude product which was purified by column chromatography (silica gel, MeOH/DCM or ethyl acetate/EtOH) to yield the desired compound.

| | | | | | Syn- | |
|---|---|---|---|---|---|---|
| | | | Car- | | thesis | |
| Ex- | | | boxylic | | accord- | |
| ample | | | acid | Amine | ing | |
| no. | Structure | Name | (ACI) | (AMN) | to | Yield |
| SC-01 | 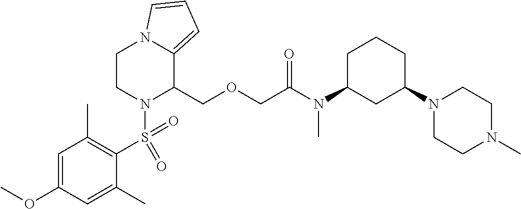 | 2-[[2-[(4-Meth- oxy-2,6-di- methylphenyl) sulfonyl]-1,2,3,4- tetrahydro-pyrrolo [1,2-a]pyrazin-1- yl]-methoxy]- N-methyl- N-[(1S,3R)-3-(4- methyl-piperazin- 1-yl)-cyclohexyl]- acetamide | ACI-01 | AMN-01 | GWI-1 | 35% |
| SC-02 | 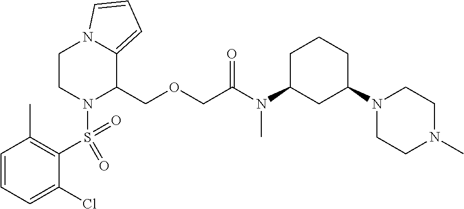 | 2-[[2-[(2-Chloro- 6-methylphenyl) sulfonyl]-1,2,3,4- tetrahydro- pyrrolo[1,2-a] pyrazin- 1-yl]-methoxy]- N-methyl-N- [(1S,3R)-3-(4- methyl-piperazin- 1-yl)-cyclohexyl]- acetamide | ACI-02 | AMN-01 | GWI-1 | 35% |
| SC-03 | 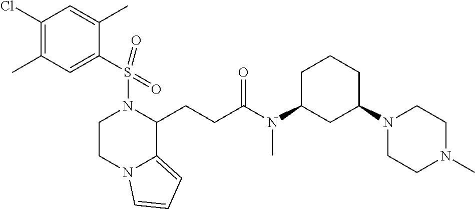 | 3-[2-[(4-Chloro- 2,5- dimethylphenyl) sulfonyl]-1,2,3,4- tetrahydro-pyrrolo [1,2-a]pyrazin-1- yl]-N-methyl-N- [(1S,3R)-3-(4- methyl-piperazin- 1-yl)-cyclohexyl]- propionamide | ACI-03 | AMN-01 | GWI-1 | 32% |
| SC-05 | 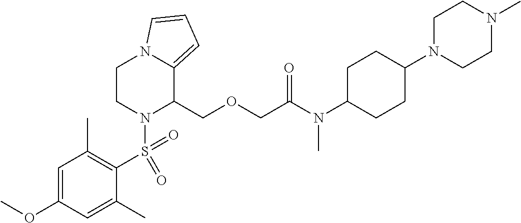 | 2-[[2-[(4- Methoxy- 2,6-dimethyl- phenyl)sulfonyl]- 1,2,3,4-tetrahydro- pyrrolo[1,2-a] pyrazin-1-yl]- methoxy]-N- methyl- N-[4-(4-methyl- piperazin-1-yl)- cyclohexyl]- acetamide | ACI-01 | AMN-03 | GWI-2 | 32% |
| SC-06 | 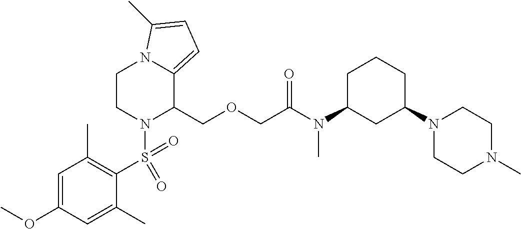 | 2-[[2-[(4- Methoxy- 2,6-dimethyl- phenyl)sulfo- nyl]-6-methyl- 1,2,3,4- tetrahydro- pyrrolo[1,2-a] pyrazin-1-yl]- methoxy]-N- methyl-N- [(1S,3R)-3-(4- methyl-piperazin- 1-yl)-cyclohexyl]- | ACI-04 | AMN-01 | GWI-1 | 38% |

### Analytical Data:

Materials and methods for HPLC-MS analytics: HPLC: Waters Alliance 2795 with PDA Waters 2998; MS: Micromass Quattro Micro™ API; Column: Waters Atlantis® T3, 3 μm, 100 Å, 2.1×30 mm; Col. temp.: 40° C., Eluent A: purified water+0.1% formic acid; Eluent B: acetonitrile (gradient grade)+0.1% formic acid; Gradient: 0% B to 100% B in 8.8 min, 100% B for 0.4 min, 100% B to 0% B in 0.01 min, 0% B for 0.8 min; Flow: 1.0 mL/min; Ionisation: ES+, 25 V; make up: 100 μL/min 70% methanol+0.2% formic acid; UV: 200-400 nm.

| Example no. | [M+] found | R.t. [min] |
|---|---|---|
| SC-01 | 602.3 | 2.9 min |
| SC-02 | 592.3 | 2.8 min |
| SC-03 | 590.3 | 3.4 min |
| SC-05 | 602.4 | 2.1 min |
| SC-06 | 616.3 | 3.3 min |

### B) Parallel Synthesis: Synthesis of Library Compounds (CC)

### 1) Synthesis of Acid Units (CC_ACI)

### Overview:

| CC_ACI unit | | |
|---|---|---|
| no. | Structure | CC_ACI name |
| CC_ACI-01 | 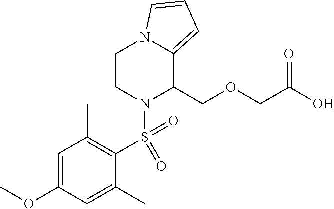 | 2-[[2-[[4-Methoxy-2,6-dimethyl- phenyl)sulfonyl]-1,2,3,4-tetrahydro- pyrrolo[1,2-a]pyrazin-1-yl]-methoxy]- acetic acid |
| CC_ACI-02 | 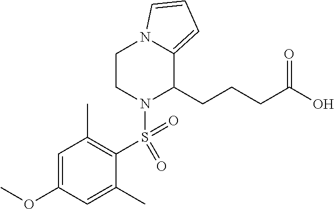 | 4-[2-[(4-Methoxy-2,6-dimethyl- phenyl)sulfonyl]-1,2,3,4-tetrahydro- pyrrolo[1,2-a]pyrazin-1-yl]-butyric acid |
| CC_ACI-03 | 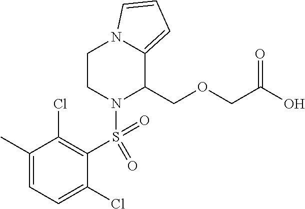 | 2-[[2-[(2,6-Dichloro-3-methyl- phenyl)sulfonyl]-1,2,3,4-tetrahydro- pyrrolo[1,2-a]pyrazin-1-yl]-methoxy]- acetic acid |
| CC_ACI-04 | 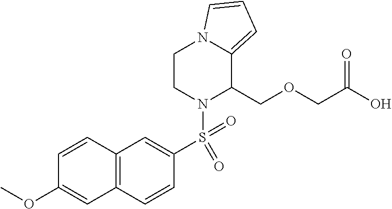 | 2-[2-[(6-Methoxy-naphthalen-2- yl)sulfonyl]-1,2,3,4-tetrahydro- pyrrolo[1,2-a]pyrazin-1-yl]-acetic acid |
| CC_ACI-05 | 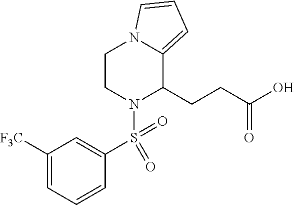 | 3-[2-[[3- (Trifluoromethyl)phenyl]sulfonyl]- 1,2,3,4-tetrahydro-pyrrolo[1,2- a]pyrazin-1-yl]-propionic acid |

Synthesis of carboxylic acid CC_ACI-01: 2-[[2-[(4-Methoxy-2,6-dimethyl-phenyl)sulfonyl]-1,2,3,4-tetrahydro-pyrrolo[1,2-a]pyrazin-1-yl]-methoxy]-acetic acid

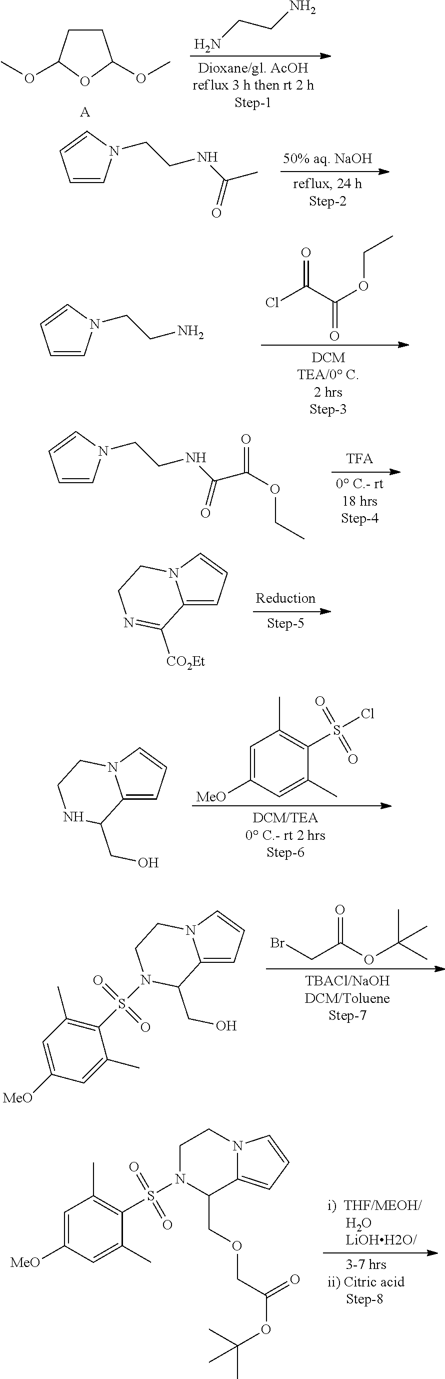

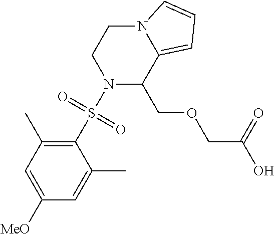

Steps 1 & 2: Glacial acetic acid (275 ml) was added slowly to a ice cold solution of 2,5-dimethoxy tetrahydro furan (50.0 g, 0.37878 mol, 1 eq) and ethylenediamine (22.727 g, 0.3787 mol, 1 eq) in dioxan (325 ml). The reaction mixture was then refluxed for 3 h and then cooled to 25° C. All the volatiles were removed under reduced pressure. The dark brown residue was then taken in 50% aqueous potassium hydroxide solution (250 ml) and refluxed for another 24 h. The reaction mixture was cooled to 0° C. and acidified by 4(N) hydrochloric acid solution. The aqueous part was washed by dichloromethane (3×100 ml). The aqueous part was now basified by 50% aqueous potassium hydroxide solution up to PH˜12 and extracted with dichloromethane (3×200 ml). The combined organic layer dried over Na₂SO₄, concentrated in reduced pressure to get the crude material, which was used for the next step reaction with out further purification. Yield: 16 g (Crude)

Step 3: To a stirring solution of step 2 compound (10 g, 0.0909 mol, 1 eq) in dichloromethane (200 ml), triethyl amine (24.746 ml, 0.1818 mol, 2 eq) was added at 0-5° C. under nitrogen atmosphere. Ethyl oxalyl chloride (10.17 ml, 0.0909 mol, 1 eq) was added very slowly to the reaction mixture at 0-5° C. After addition the reaction mixture was stirred for another 2 h at 0-5° C. The reaction mixture was diluted with dichloromethane (100 ml) and washed by water (2×50 ml) followed by brine (50 ml). The organic layer dried over Na₂SO₄, concentrated in reduced pressure to get the crude material, which was purified by silica (100-200) column chromatography. The compound eluted by 30% ethyl acetate/hexane. Yield: 55% (10.5 gm, 0.02336 mol)

Step 4: Step 4 compound (10.0 g, 0.0476 mol, 1 eq) was taken in 250 ml round bottled flux fitted with a nitrogen balloon, cooled to 0° C. Trifluoro acetic acid (100 ml) was added slowly by dropping funnel to compound and then stirred for 18 h at room temperature. Evaporated all the trifluoro acetic acid under reduced pressure, the residue was dissolved in dichloromethane (100 ml) and neutralized by slowly addition of saturated sodium hydrogen carbonate solution until P^{H} become basic. Organic layer was separated and the aqueous layer was extracted with dichloromethane (100 ml). The combined organic layer dried over Na₂SO₄, concentrated in reduced pressure to get the crude material, which was purified by silica (100-200) column chromatography. The compound eluted by 50% ethyl acetate/hexane. Yield: 87.5% (8.0 g, 0.04166 mol)

Step 5: To a cold stirring suspension of lithium aluminum hydride (0.99 g, 0.026 mol, 2 eq) in tetrahydro furan (45 ml) was added drop wise a solution of step 4 compound (2.6 gm, 0.01354 mol, 1 eq) in tetrahydro furan (45 ml) by addition funnel at 0° C. After addition the reaction mixture was stirred for 2 h at room temperature. The reaction mixture again cooled to 0° C. and the reaction mixture was quenched by saturated Na₂SO₄ solution. After quenching the reaction mixture was stirred with ethyl acetate (100 ml) for 20 min and then filtered through celite pad and washed by with ethyl acetate (100 ml). The organic layer concentrated in reduced pressure to get the pure product. Yield: 97.1% (2.0 gm, 0.01315 mol)

Step 6: To a stirring solution of step 5 compound (2.0 g, 0.0315 mol, 1 eq) in dichloromethane (90 ml) was added triethyl amine (4.45 ml, 0.03275 mol, 2.5 eq). The reaction mixture was then cooled to 0-50 C and stirred for 10 min. 4-Methoxy-2,6-dimethyl-benzene sulfonyl chloride (3.7 gm, 0.01578 mol, 1.2 eq) was added. The reaction mixture then stirred for 14 h at room temperature. The reaction mixture was diluted with dichloromethane (100 ml) and washed by water (2×50 ml) followed by brine (50 ml). The organic layer dried over Na₂SO₄, concentrated in reduced pressure to get the crude material, which was purified by silica (100-200) column chromatography. The compound eluted by 40% ethyl acetate/hexane. Yield: 42.9% (2.1 gm, 0.00563 mol)

Step 7: To a stirring solution of step 6 compound (3 g, 0.00805 mol, 1 eq) in dichloromethane & toluene (1:1) (32.7 ml), tetra butyl ammonium chloride (0.8214 g, 0.0029559 mol, 0.82144 eq) was added. The reaction mixture then cooled to 0° C. and sodium hydroxide solution (35%) (32.7 ml) and tert-butyl bromoacetate (1.96 ml, 0.01328 mol, 1.65 eq) was added drop wise. The reaction mixture then stirred for 14 h at room temperature. The organic phase was separated and washed by water (2×20 ml) followed by brine (20 ml). The organic layer dried over Na₂SO₄, concentrated in reduced pressure to get the crude material, which was purified by silica (100-200) column chromatography. The compound eluted by 20% ethyl acetate/hexane. Yield: 58.75% (2.2 g, 0.00473 mol)

Step 8: To a stirring solution of step 7 compound (1.8 g, 0.003874 mol, 1 eq) in a mixture of tetrahydrofuran, methanol and water (4:2:1) (27 ml) was added lithium hydroxide mono hydrate (0.44 g, 0.011623 mol, 3 eq). The reaction mixture then stirred for 4 h at room temperature. Evaporate all the solvent. The residue then dissolved in water (100 ml). The aqueous part then washed by ethyl acetate (50 ml). The aqueous part then acidified by citric acid solution (10%). The aqueous part was extracted by ethyl acetate (2×50 ml). The combined organic layer dried over Na₂SO₄, concentrated in reduced pressure to get the pure product. Yield: 81.9% (1.2 g, 0.00317 mol)

Synthesis of carboxylic acid CC_ACI-02: 4-[2-[(4-Methoxy-2,6-dimethyl-phenyl)sulfonyl]-1,2,3,4-tetrahydro-pyrrolo[1,2-a]pyrazin-1-yl]-butyric acid

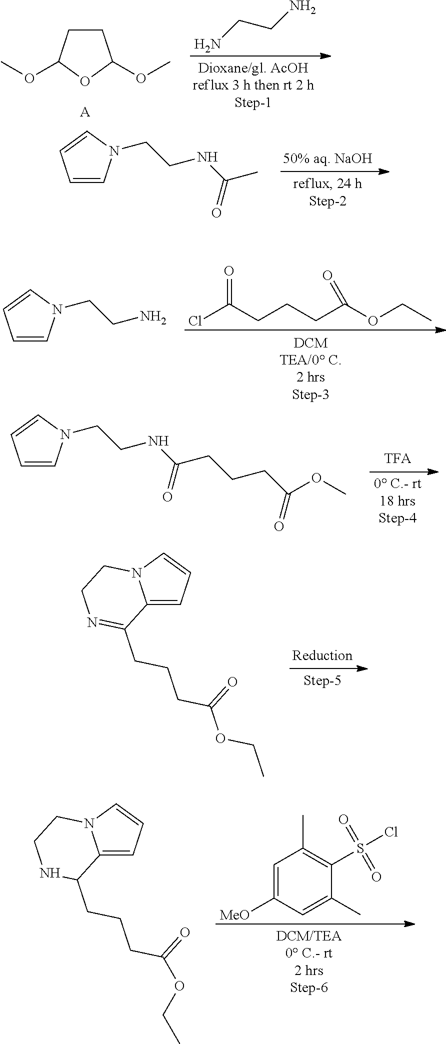

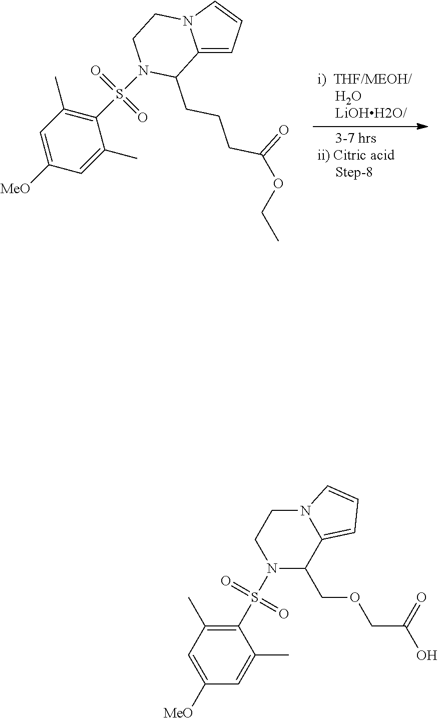

### Steps 1 & 2: see CC_ACI-01

Step 3: To a stirring solution of step 2 compound (8.7 g, 0.07909 mol, 1 eq) in dichloromethane (170 ml), triethyl amine (21.53, 0.15818 mol, 2 eq) was added at 0-5° C. under nitrogen atmosphere. Ethyl glutaryl chloride (11.25, 0.07909 mol, 1 eq) was added very slowly to the reaction mixture at 0-5° C. After addition the reaction mixture was stirred for another 2 h at 0-5° C. The reaction mixture was diluted with dichloromethane (100 ml) and washed by water (2×50 ml) followed by brine (50 ml). The organic layer dried over Na₂SO₄, concentrated in reduced pressure to get the crude material, which was purified by silica (100-200) column chromatography. The compound eluted by 40% ethyl acetate/hexane. Yield: 50% (10 gm, 0.0396 mol)

Step 4: Step 4 compound (10.0 gm, 0.03965 mol, 1 eq) was taken in 250 ml round bottled flux fitted with a nitrogen balloon, cooled to 0° C. Trifluoro acetic acid (100 ml) was added slowly by dropping funnel to compound and then stirred for 18 h at room temperature. Evaporated all the trifluoro acetic acid under reduced pressure, the residue was dissolved in dichloromethane (100 ml) and neutralized by slowly addition of saturated sodium hydrogen carbonate solution until P^{H} become basic. Organic layer was separated and the aqueous layer was extracted with dichloromethane (100 ml). The combined organic layer dried over Na₂SO₄, concentrated in reduced pressure to get the crude material, which was purified by silica (100-200) column chromatography. The compound eluted by 50% ethyl acetate/hexane. Yield: 32.33% (3.0 gm, 0.0128 mol)

Step 5: The solution of step 4 compound (3 g, 0.0128 mol, 1 eq) in ethanol (30 ml) was deoxygenated well by argon. Pd/C (10%) (0.3 gm) was then added to the reaction mixture under argon atmosphere. The reaction mixture finally degassed by hydrogen and stirred under hydrogen for 14 h at room temperature. The reaction mixture was filtered through celite pad and washed by ethanol (50 ML). The organic part concentrated in reduced pressure to get the crude material which was purified by silica (100-200) column chromatography. The compound eluted by 3% methanol/dichloromethane. Yield: 86.12% (2.8 gm, 0.011 mol)

Step 6: To a stirring solution of step 5 compound (2.0 gm, 0.00847 mol, 1 eq) in dichloromethane (43 ml) was added triethyl amine (2.88 ml, 0.0211755 mol, 2.5 eq). The reaction mixture was then cooled to 0-50 C and stirred for 10 min. 4-Methoxy-2,6-dimethyl-benzene sulfonyl chloride (2.386 gm, 0.010169 mol, 1.2 eq) was added. The reaction mixture then stirred for 14 h at room temperature. The reaction mixture was diluted with dichloromethane (50 ml) and washed by water (2×50 ml) followed by brine (50 ml). The organic layer dried over Na₂SO₄, concentrated in reduced pressure to get the crude material, which was purified by silica (100-200) column chromatography. The compound eluted by 40% ethyl acetate/hexane. Yield: 81.61% (3 gm, 0.06912 mol)

Step 7: To a stirring solution of step 7 compound (1.8 gm, 0.003874 mol, 1 eq) in a mixture of tetrahydrofuran, methanol and water (4:2:1) (27 ml) was added lithium hydroxide mono hydrate (0.44 gm, 0.011623 mol, 3 eq). The reaction mixture then stirred for 4 h at room temperature. Evaporate all the solvent. The residue then dissolved in water (100 ml). The aqueous part then washed by ethyl acetate (50 ml). The aqueous part then acidified by citric acid solution (10%). The aqueous part was extracted by ethyl acetate (2×50 ml). The combined organic layer dried over Na₂SO₄, concentrated in reduced pressure to get the pure product. Yield: 46.33% (1.3 gm, 0.032 mol)

Synthesis of carboxylic acid CC_ACI-03: 2-[[2-[(2,6-Dichloro-3-methyl-phenyl)sulfonyl]-1,2,3,4-tetrahydro-pyrrolo[1,2-a]pyrazin-1-yl]-methoxy]-acetic acid
Synthesis of CC_ACI-03 was carried out in analogy to CC_ACI-01 utilizing 2,6-dichloro-3-methylbenzene-1-sulfonyl chloride.
Synthesis of carboxylic acid CC_ACI-04: 2-[2-[(6-Methoxy-naphthalen-2-yl)sulfonyl]-1,2,3,4-tetrahydro-pyrrolo[1,2-a]pyrazin-1-yl]-acetic acid

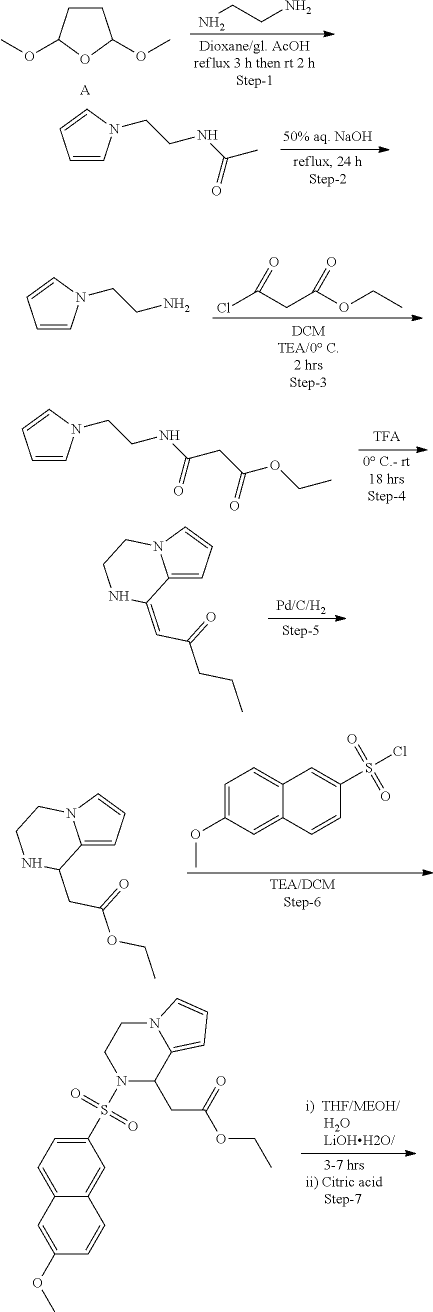

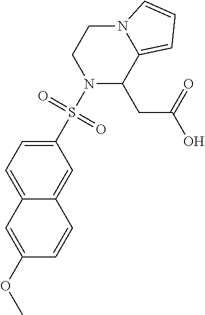

### Steps 1 & 2: see CC_ACI-01

Step 3: To a stirring solution of step 2 compound (6 g, 0.05454 mol, 1 eq) in dichloromethane (120 ml), triethyl amine (14.84 ml, 0.109 mol, 2 eq) was added at 0-5° C. under nitrogen atmosphere. Ethyl manolyl chloride (8.212, 0.05454 mol, 1 eq) was added very slowly to the reaction mixture at 0-5° C. After addition the reaction mixture was stirred for another 2 h at 0-5° C. The reaction mixture was diluted with dichloromethane (100 ml) and washed by water (2×50 ml) followed by brine (50 ml). The organic layer dried over Na₂SO₄, concentrated in reduced pressure to get the crude material, which was purified by silica (100-200) column chromatography. The compound eluted by 40% ethyl acetate/hexane. Yield: 45% (5.5 gm, 0.02455 mol)

Step 4: Step 4 compound (5.5 gm, 0.024557 mol, 1 eq) was taken in 250 ml round bottled flux fitted with a nitrogen balloon, cooled to 0° C. Trifluoro acetic acid (55 ml) was added slowly by dropping funnel to compound and then stirred for 18 h at room temperature. Evaporated all the trifluoro acetic acid under reduced pressure, the residue was dissolved in dichloromethane (100 ml) and neutralized by slowly addition of saturated sodium hydrogen carbonate solution until P^{H} become basic. Organic layer was separated and the aqueous layer was extracted with dichloromethane (100 ml). The combined organic layer dried over Na₂SO₄, concentrated in reduced pressure to get the crude material, which was purified by silica (100-200) column chromatography. The compound eluted by 50% ethyl acetate/hexane. Yield: 53.9% (2.7 gm, 0.01323 mol)

Step 5: The solution of step 4 compound (2.7 g, 0.0131 mol, 1 eq) in ethanol (27 ml) was deoxygenated well by argon. Pd/C (10%) (0.27 gm) was then added to the reaction mixture under argon atmosphere. The reaction mixture finally degassed by hydrogen and stirred under hydrogen for 14 h at room temperature. The reaction mixture was filtered through celite pad and washed by ethanol (30 ML). The organic part concentrated in reduced pressure to get the crude material which was purified by silica (100-200) column chromatography. The compound eluted by 3% methanol/dichloromethane. Yield: 66% (1.8 gm, 0.00865 mol)

Step 6: To a stirring solution of step 5 compound (1.7 gm, 0.00817 mol, 1 eq) in dichloromethane (41 ml) was added triethyl amine (2.78 ml, 0.02042 mol, 2.5 eq). The reaction mixture was then cooled to 0-5° C. and stirred for 10 min. 6-methoxy-naphthalene-2-sulfonyl chloride (2.517 gm, 0.0098 mol, 1.2 eq) was added. The reaction mixture then stirred for 14 h at room temperature. The reaction mixture was diluted with dichloromethane (50 ml) and washed by water (2×30 ml) followed by brine (30 ml). The organic layer dried over Na₂SO₄, concentrated in reduced pressure to get the crude material, which was purified by silica (100-200) column chromatography. The compound eluted by 40% ethyl acetate/hexane. Yield: 77.1% (2.9 gm, 0.0063 mol)

Step 7: To a stirring solution of step 7 compound (3.2 gm, 0.00746 mol, 1 eq) in a mixture of tetrahydrofuran, methanol and water (4:2:1) (52 ml) was added lithium hydroxide mono hydrate (0.94 gm, 0.0224 mol, 3 eq). The reaction mixture then stirred for 4 h at room temperature. Evaporate all the solvent. The residue then dissolved in water (100 ml). The aqueous part then washed by ethyl acetate (30 ml). The aqueous part then acidified by citric acid solution (10%). The aqueous part was extracted by ethyl acetate (2×50 ml). The combined organic layer dried over Na₂SO₄, concentrated in reduced pressure to get the pure product. Yield: 62.5% (1.9 gm, 0.00475 mol)

Synthesis of carboxylic acid CC_ACI-05: 3-[2-[[3-(Trifluoromethyl)phenyl]sulfonyl]-1,2,3,4-tetrahydro-pyrrolo[1,2-a]pyrazin-1-yl]-propionic acid

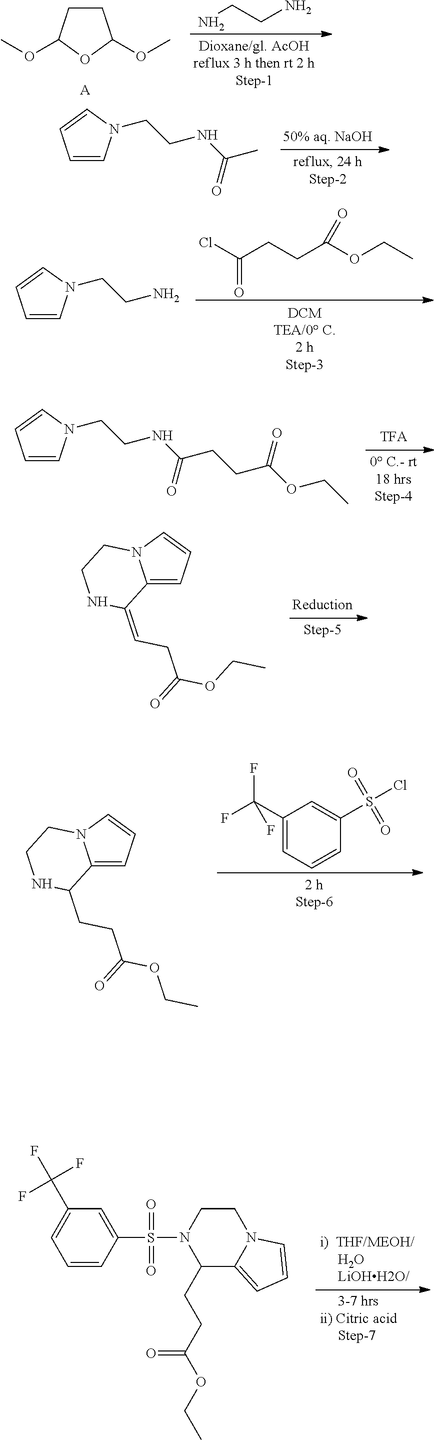

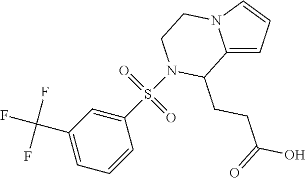

### Steps 1 & 2: see CC_ACI-01

Step 3: To a stirring solution of step 2 compound (8.1 g, 0.0736 mol, 1 eq) in dichloromethane (160 ml), triethyl amine (20, 0.1472 mol, 2 eq) was added at 0-5° C. under nitrogen atmosphere. Ethyl succinyl chloride (13.155, 0.0736 mol, 1 eq) was added very slowly to the reaction mixture at 0-5° C. After addition the reaction mixture was stirred for another 2 h at 0-5° C. The reaction mixture was diluted with dichloromethane (100 ml) and washed by water (2×50 ml) followed by brine (50 ml). The organic layer dried over Na₂SO₄, concentrated in reduced pressure to get the crude material, which was purified by silica (100-200) column chromatography. The compound eluted by 40% ethyl acetate/hexane. Yield: 51.37% (9.0 gm, 0.03781 mol)

Step 4: Step 4 compound (9.0 gm, 0.0377 mol, 1 eq) was taken in 250 ml round bottled flux fitted with a nitrogen balloon, cooled to 0° C. Trifluoro acetic acid (90 ml) was added slowly by dropping funnel to compound and then stirred for 18 h at room temperature. Evaporated all the trifluoro acetic acid under reduced pressure, the residue was dissolved in dichloromethane (100 ml) and neutralized by slowly addition of saturated sodium hydrogen carbonate solution until P″ become basic. Organic layer was separated and the aqueous layer was extracted with dichloromethane (100 ml). The combined organic layer dried over Na₂SO₄, concentrated in reduced pressure to get the crude material, which was purified by silica (100-200) column chromatography. The compound eluted by 50% ethyl acetate/hexane. Yield: 16.87% (1.4 gm, 0.006363 mol)

Step 5: The solution of step 4 compound (1.4 g, 0.006363 mol, 1 eq) in ethanol (14 ml) was deoxygenated well by argon. Pd/C (10%) (0.14 gm) was then added to the reaction mixture under argon atmosphere. The reaction mixture finally degassed by hydrogen and stirred under hydrogen for 14 h at room temperature. The reaction mixture was filtered through celite pad and washed by ethanol (20 ML). The organic part concentrated in reduced pressure to get the crude material which was purified by silica (100-200) column chromatography. The compound eluted by 3% methanol/dichloromethane. Yield: 92% (1.3 gm, 0.005855 mol)

Step 6: To a stirring solution of step 5 compound (1.3 gm, 0.00585 mol, 1 eq) in dichloromethane (30 ml) was added triethyl amine (2 ml, 0.01465 mol, 2.5 eq). The reaction mixture was then cooled to 0-50 C and stirred for 10 min. 3-trifluoromethyl-benzene sulfonyl chloride (1.718 gm, 0.00702 mol, 1.2 eq) was added. The reaction mixture then stirred for 14 h at room temperature. The reaction mixture was diluted with dichloromethane (30 ml) and washed by water (2×20 ml) followed by brine (20 ml). The organic layer dried over Na₂SO₄, concentrated in reduced pressure to get the crude material, which was purified by silica (100-200) column chromatography. The compound eluted by 40% ethyl acetate/hexane. Yield: 55.65% (1.4 gm, 0.003255 mol)

Step 7: To a stirring solution of step 7 compound (1.4 gm, 0.003255 mol, 1 eq) in a mixture of tetrahydrofuran, methanol and water (4:2:1) (23 ml) was added lithium hydroxide mono hydrate (0.3411 gm, 0.00976 mol, 3 eq). The reaction mixture then stirred for 4 h at room temperature. Evaporate all the solvent. The residue then dissolved in water (50 ml). The aqueous part then washed by ethyl acetate (30 ml). The aqueous part then acidified by citric acid solution (10%). The aqueous part was extracted by ethyl acetate (2×50 ml). The combined organic layer dried over Na₂SO₄, concentrated in reduced pressure to get the pure product. Yield: 90.4% (1.2 gm, 0.00294 mol)

### 2) Synthesis of the Amine Units (CC_AMN):

### Overview:

| AMN-CC unit | | |
|---|---|---|
| no. | Structure | AMN-CC name |
| CC_AMN-01 | 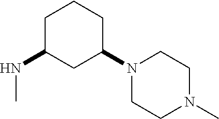 | Methyl-[cis-3-(4-methyl-piperazin-1- yl)-cyclohexyl]-amine |
| CC_AMN-02 | 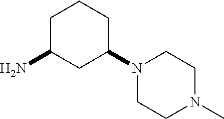 | [cis-3-(4-Methyl-piperazin-1-yl)- cyclohexyl]-amine |
| CC_AMN-04 | 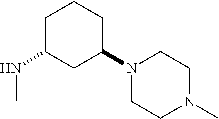 | Methyl-[trans-3-(4-methyl-piperazin-1- yl)-cyclohexyl]-amine |
| CC_AMN-05 | 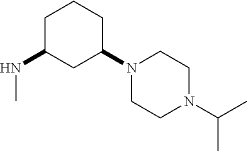 | [cis-3-(4-Isopropyl-piperazin-1-yl)- cyclohexyl]-methyl-amine |
| CC_AMN-06 | 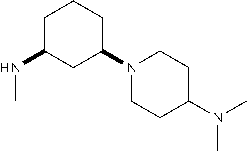 | Dimethyl-[1-[cis-3-methylamino- cyclohexyl]-piperidin-4-yl]-amine |
| CC_AMN-07 | 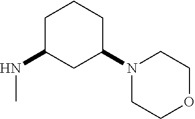 | Methyl-[cis-3-morpholin-4-yl- cyclohexyl]-amine |
| CC_AMN-08 | 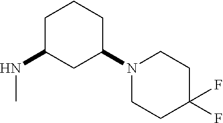 | [cis-3-(4,4-Difluoro-piperidin-1-yl)- cyclohexyl]-methyl-amine |
| CC_AMN-09 | 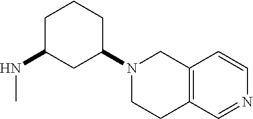 | Methyl-[cis-3-(1,2,3,4-tetrahydro- [2,6]naphthyridin-2-yl)-cyclohexyl]- amine |

### Overview—General Synthesis:

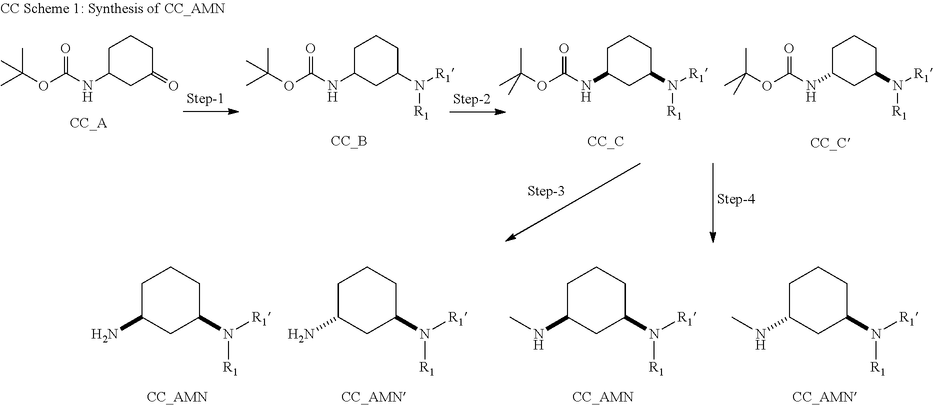

### Step 1:

The reaction is carried out in analogy to WO 2009/021944 example 1e). The required amine (5 equiv.), commercially available tert-butyl 3-oxocyclohexylcarbamate (CC_A) (1 equiv.) and acidic acid (10 equiv.) are dissolved in methanol and stirred at room temperature for 30 minutes. Sodium triacetoxy borohydride (2 equiv.) is added portionwise and the reaction mixture stirred at room temperature for 2 hours. Subsequently the reaction is quenched with sodium hydrogencarbonate and extracted with dichloromethane. The solvent is evaporated and the desired product CC_B obtained after column chromatography.

### Step 2:

Cis- and trans-diastereomers of CC_C and CC_D are separated either via HPLC chromatography or crystallization. Pure cis-diastereomers and trans-diastereomers are obtained.

### Step 3:

Acetylchloride (3 equiv.) is added to a solution of CC_B in ethanol and stirred overnight. The solvent is removed under reduced pressure, the residue taken up in aq. sodium hydroxide and extracted with dichloromethane. The desired product CC_D is obtained after removal of dichloromethane and column chromatography.

### Step 4:

The reaction is carried out in analogy to WO 2009/021944 example 1f).

A solution of CC_B (1 equiv.) in tetrahydrofuran is slowly added to a solution of Lithiomaluminiumhydride (1 M, Toluene, 3 equiv.) in THF at room temperature. The reaction mixture is heated to 75° C. for 2 hours, subsequently quenced with aq. sodium hydroxide and water. The precipitate is filtered of and the reaction solution concentrated to obtain the desired product CC_C.

| AMN-CC unit No. | Name | Amine utilized | Synthesis |
|---|---|---|---|
| CC_AMN-01 | Methyl-[cis-3-(4-methyl-piperazin- | 1-Methyl-piperazine | Step 1, Step 2 & Step 4 |
| | 1-yl)-cyclohexyl]-amine | | |
| CC_AMN-02 | [cis-3-(4-Methyl-piperazin-1-yl)- | 1-Methyl-piperazine | Step 1, Step 2 & Step 3 |
| | cyclohexyl]-amine | | |
| CC_AMN-04 | Methyl-[trans-3-(4-methyl- | 1-Methyl-piperazine | Step 1, Step 2 & Step 4 |
| | piperazin-1-yl)-cyclohexyl]-amine | | |
| CC_AMN-05 | [cis-3-(4-Isopropyl-piperazin-1-yl)- | 1-Isopropyl-piperazine | Step 1, Step 2 & Step 4 |
| | cyclohexyl]-methyl-amine | | |
| CC_AMN-06 | Dimethyl-[1-[cis-3-methylamino- | Dimethyl-piperidin-4-yl- | Step 1, Step 2 & Step 4 |
| | cyclohexyl]-piperidin-4-yl]-amine | amine | |
| CC_AMN-07 | Methyl-[cis-3-morpholin-4-yl- | Morpholine | Step 1, Step 2 & Step 4 |
| | cyclohexyl]-amine | | |
| CC_AMN-08 | [cis-3-(4,4-Difluoro-piperidin-1-yl)- | 4,4-Difluoro-piperidine | Step 1, Step 2 & Step 4 |
| | cyclohexyl]-methyl-amine | | |
| CC_AMN-09 | Methyl-[cis-3-(1,2,3,4-tetrahydro- | 1,2,3,4-Tetrahydro- | Step 1, Step 2 & Step 4 |
| | [2,6]naphthyridin-2-yl)-cyclohexyl]- | [2,6]naphthyridine | |
| | amine | | |
| CC_AMN-05_Mix | [3-(4-Isopropyl-piperazin-1-yl)- | | |
| | cyclohexyl]-methyl-amine | | |
| CC_AMN-07_Mix | Methyl-[3-morpholin-4-yl- | | |
| | cyclohexyl]-amine | | |
| CC_AMN-11 | Tert-butyl methyl((1s,3s)-3-(4- | | |
| | methylpiperazin-1- | | |
| | yl)cyclobutyl)carbamate | | |

Synthesis of: Methyl-[cis-3-(4-methyl-piperazin-1-yl)-cyclohexyl]-amine (CC_AMN-01)

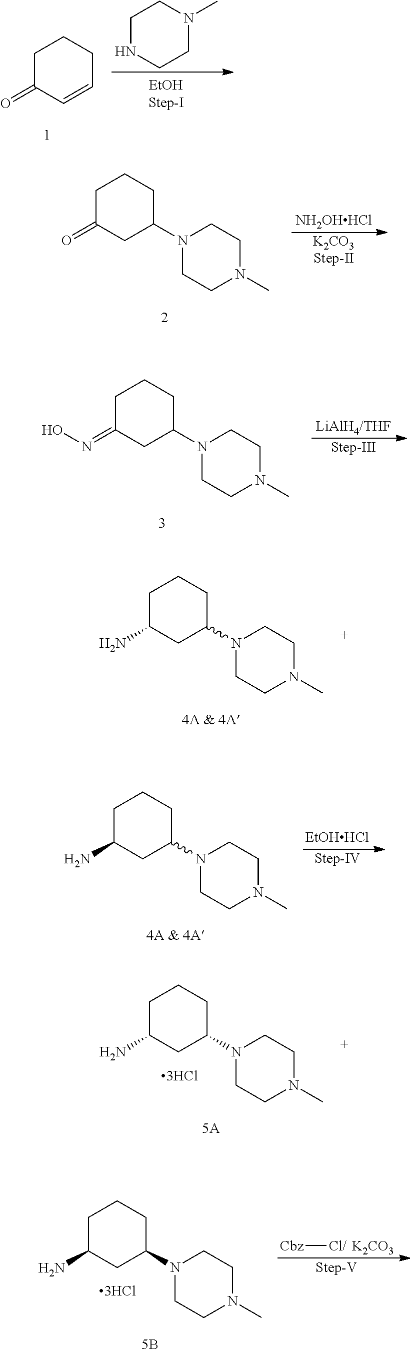

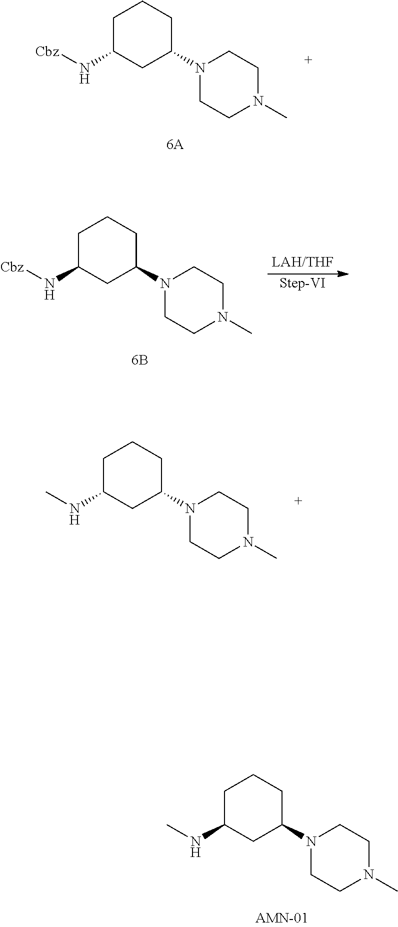

Steps I & II: To the stirred solution of 2-cyclohexene-1-one (100.0 g, 1.0416 mol 1.0 eq.) in ethanol (300 ml) was added drop-wise a solution of N-methylpiperazine (104.16 g, 1.0416 mol, 1.0 eq.) and the reaction mixture was stirred at RT for 5 h. Then the reaction mixture was diluted with 600 ml of ethanol and cool to 0° C. and K₂CO₃ (172.48 g, 1.2499 mol, 1.2 eq.) was added followed by the addition of NH₂OH.HCl (86.7 g, 1.2499 mol, 1.2 eq.) in portions. The stirring was continued at 0° C. for 30 min and then at RT for 16 h. The reaction mixture was filtered through celite bed and washed with ethanol (500 ml), and filtrate was concentrated under reduced pressure. The concentrated mass was stirred with THF (100 ml) and n-hexane (500 ml) for 3 h and then filtered to get desired compound 3 as white solid which was used in the next step without further purification. Yield: 54% (120.0 g, 0.5687 mol).

Step III: To the stirred suspension of LAH (13.15 g, 0.355 mol, 2.5 eq.) in THF (600 ml) was added portion-wise compound 3 (30.0 g, 0.142 mol, 1.0 eq.) at 0° C. After the complete addition, the reaction mixture was warmed to RT and then refluxed for 14 h. The reaction mixture was cooled to 0° C. and quenched with 15% NaOH solution (15 ml). The reaction mixture was filtered and washed with 10% MeOH/DCM (500 ml). The filtrate was concentrated under reduced pressure to afford crude compound 4 as sticky solid. Yield: 67% (19.0 g, 0.964 mol).

Step IV: To the stirred solution of compound 4 (30.0 g, 0.10 mol) in methanol (70 ml) was added drop-wise ethanolic/HCl (7N, 150 ml) at 50° C. and the reaction mixture was stirred at same temperature for 3 h. The reaction mixture was cooled to RT and solid mass was separated by filtration. The solid residue was recrystallized with methanol to get hydrochloride salt of the amine 5.

Yield: 32% (2.8 g, 0.009 mol).

Step V: To the mixture of compound 5 (5.0 g, 0.016 mol, 1.0 eq.) and K₂CO₃ (9.05 g, 65.56 mol, 4.0 eq.) in water (15 ml) was added a solution of Cbz-Cl (2.85 g, 16.39 mol, 1.0 eq.) in toluene (25 ml) at 0° C. and then the reaction mixture was stirred at RT for 16 h. The reaction mixture was diluted with water (15 ml) and layers are separated. Organic layer was dried over sodium sulfate and concentrated to get crude product which was purified by column chromatography (neutral alumina; 1% MeOH/DCM) to yield compound 6. Yield: 56% (3.2 g, 0.009 mol)

Step VI: A solution of compound 8 (3.0 g, 0.009 mol, 1.0 eq.) in THF (10 ml) was added drop-wise to the suspension of LAH (1.4 g, 0.036 mol, 4.0 eq.) in THF (30 ml) at 0° C. After complete addition, the reaction mixture was warmed to RT and then refluxed for 30 min. The reaction mixture was cooled to 0° C. and quenched with 10% NaOH solution (1.4 ml). The reaction mixture was filtered and washed with THF (2×50 ml), filtrate was concentrated under reduced pressure to afford desired amine as colorless sticky liquid. Yield: 89% (1.7 g, 0.008 mol)

CC_AMN-01 was synthesized as a mixture of two cis isomers.

Synthesis of: [Cis-3-(4-Methyl-piperazin-1-yl)-cyclohexyl]-amine (CC_AMN-02)

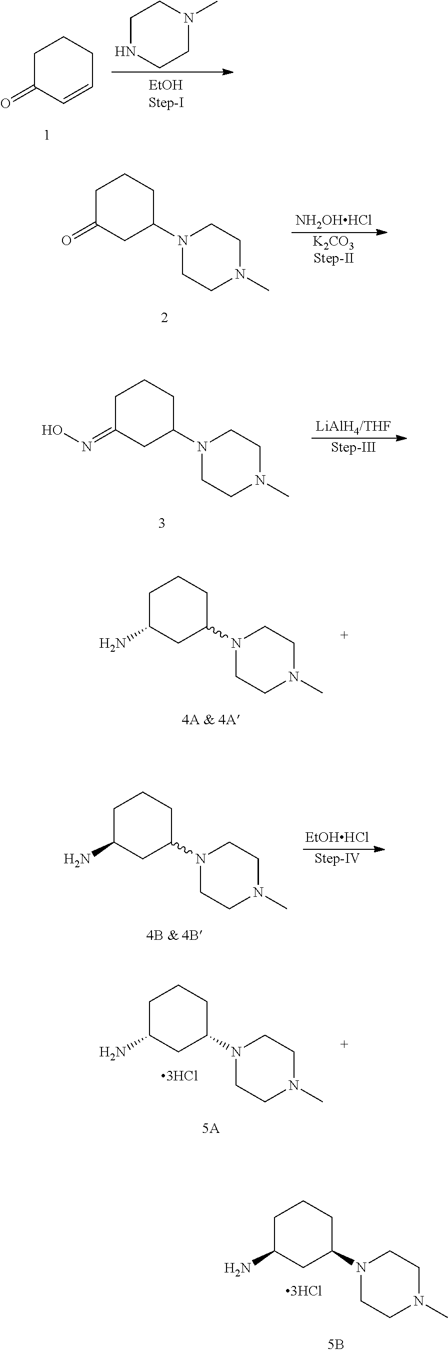

Steps I & II: To the stirred solution of 2-cyclohexene-1-one (100.0 g, 1.0416 mol 1.0 eq.) in ethanol (300 ml) was added drop-wise a solution of N-methylpiperazine (104.16 g, 1.0416 mol, 1.0 eq.) and the reaction mixture was stirred at RT for 5 h. Then the reaction mixture was diluted with 600 ml of ethanol and cool to 0° C. and K₂CO₃ (172.48 g, 1.2499 mol, 1.2 eq.) was added followed by the addition of NH₂OH.HCl (86.7 g, 1.2499 mol, 1.2 eq.) in portions. The stirring was continued at 0° C. for 30 min and then at RT for 16 h. The reaction mixture was filtered through celite bed and washed with ethanol (500 ml), and filtrate was concentrated under reduced pressure. The concentrated mass was stirred with THF (100 ml) and n-hexane (500 ml) for 3 h and then filtered to get desired compound 3 as white solid which was used in the next step without further purification. Yield: 54% (120.0 g, 0.5687 mol).

Step III: To the stirred suspension of LAH (13.15 g, 0.355 mol, 2.5 eq.) in THF (600 ml) was added portion-wise compound 3 (30.0 g, 0.142 mol, 1.0 eq.) at 0° C. After the complete addition, the reaction mixture was warmed to RT and then refluxed for 14 h. The reaction mixture was cooled to 0° C. and quenched with 15% NaOH solution (15 ml). The reaction mixture was filtered and washed with 10% MeOH/DCM (500 ml). The filtrate was concentrated under reduced pressure to afford crude compound 4 as sticky solid. Yield: 67% (19.0 g, 0.964 mol).

Step IV: To the stirred solution of compound 4 (30.0 g, 0.10 mol) in methanol (70 ml) was added drop-wise ethanolic/HCl (7N, 150 ml) at 50° C. and the reaction mixture was stirred at same temperature for 3 h. The reaction mixture was cooled to RT and solid mass was separated by filtration. The solid residue was recrystallized with methanol to get hydrochloride salt of the amine 5. Yield: 9.0% (2.8 g, 0.009 mol)

CC_AMN-02 was synthesized as a mixture of two cis isomers.

Synthesis of: [3-(4-Isopropyl-piperazin-1-yl)-cyclohexyl]-methyl-amine (CC_AMN-05_Mix)

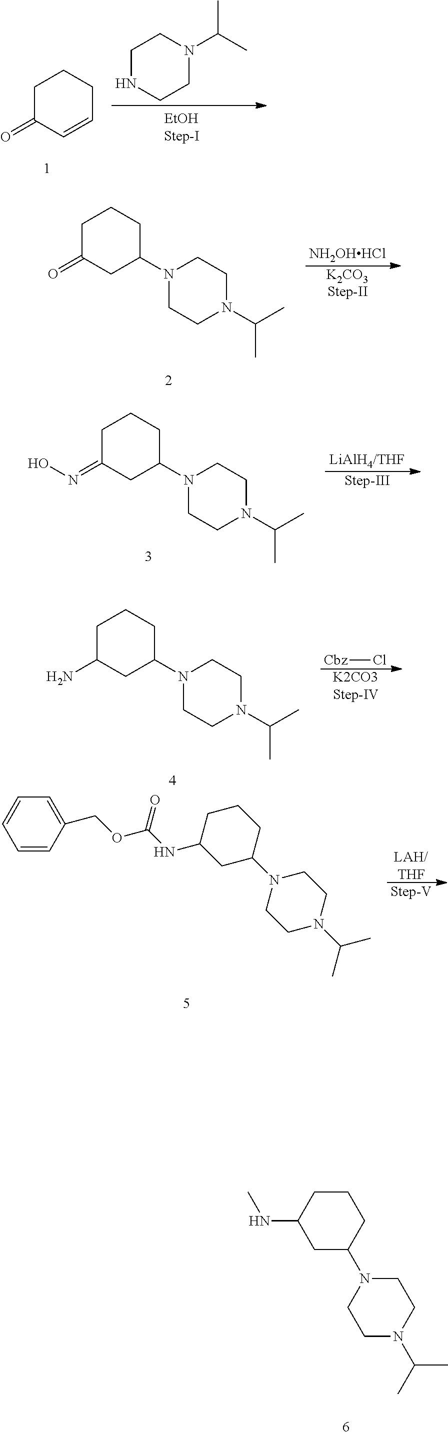

Steps I & II: To the stirred solution of 2-cyclohexene-1-one (15.0 g, 0.15 mol 1.0 eq.) in ethanol (300 ml) was added drop-wise a solution of N-Isopropylpiperazine (16 g, 0.125 mol, 0.8 eq.) and the reaction mixture was stirred at RT for 5 h. Then the reaction mixture was diluted with 200 ml of ethanol and cool to 0° C. and K₂CO₃ (27.52 g, 0.19 mol, 1.2 eq.) was added followed by the addition of NH₂OH.HCl (13.11 g, 0.19 mol, 1.2 eq.) in portions. The stirring was continued at 0° C. for 30 min and then at RT for 16 h. The reaction mixture was filtered through celite bed and washed with ethanol (500 ml), and filtrate was concentrated under reduced pressure. The concentrated mass was stirred with THF (100 ml) and n-hexane (500 ml) for 3 h and then filtered to get desired compound 3 as white solid which was used in the next step without further purification. Yield: 64% (23.0 g, 0.096 mol)

Step III: To the stirred suspension of LAH (7.6 g, 0.20 mol, 2.0 eq.) in THF (300 ml) was added portion-wise compound 3 (25 g, 0.10 mol, 1.0 eq.) at 0° C. After the complete addition, the reaction mixture was warmed to RT and then refluxed for 24 h. The reaction mixture was cooled to 0° C. and quenched with 15% NaOH solution (15 ml). The reaction mixture was filtered and washed with 10% MeOH/DCM (500 ml). The filtrate was concentrated under reduced pressure to afford crude compound 4 as sticky solid. Yield: 67% (19.0 g, 0.964 mol).

Step IV: To the mixture of compound 4 (1.0 g, 0.004 mol, 1.0 eq.) and K₂CO₃ (2.7 g, 0.019 mol, 4.0 eq.) in water (20 ml) was added a solution of Cbz-Cl (0.816 g, 0.004 mol, 1.0 eq.) in toluene (20 ml) at 0° C. and then the reaction mixture was stirred at RT for 16 h. The reaction mixture was diluted with water (15 ml) and layers are separated. Organic layer was dried over sodium sulfate and concentrated to get crude product which was purified by column chromatography (neutral alumina; 1% MeOH/DCM) to yield compound 5. Yield: 50% (0.80 g, 0.002 mol).

Step V: A solution of compound 5 (0.60 g, 0.0016 mol, 1.0 eq.) in THF (10 ml) was added drop-wise to the suspension of LAH (0.254 g, 0.0066 mol, 4.0 eq.) in THF (10 ml) at 0° C. After complete addition, the reaction mixture was warmed to RT and then refluxed for 30 min. The reaction mixture was cooled to 0° C. and quenched with 10% NaOH solution (0.25 ml). The reaction mixture was filtered and washed with THF (2×50 ml), filtrate was concentrated under reduced pressure to afford desired amine as colorless sticky liquid. Yield: 91% (0.35 g, 0.0014 mol)

AMN-05 was synthesized as mixture of 4 isomers (2cis+2 trans).

Synthesis of: Methyl-[3-morpholin-4-yl-cyclohexyl]-amine (CC_AMN-07_Mix)

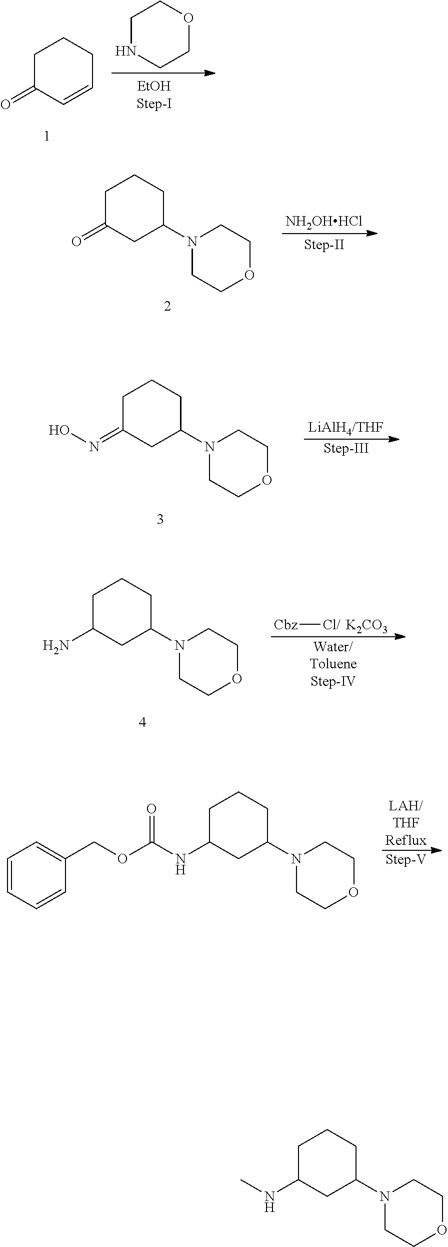

Steps I & II: To the stirred solution of 2-cyclohexene-1-one (5.0 g, 0.052 mol 1.0 eq.) in ethanol (50 ml) was added drop-wise a solution of morpholine (4.5 ml, 0.052 mol, 1.0 eq.) and the reaction mixture was stirred at RT for 5 h. Then the reaction mixture was diluted with 100 ml of ethanol and cool to 0° C. and K₂CO₃ (8.04 g, 0.0058 mol, 1.12 eq.) was added followed by the addition of NH₂OH.HCl (4.02 g, 0.0058 mol, 1.12 eq.) in portions. The stirring was continued at 0° C. for 30 min and then at RT for 16 h. The reaction mixture was filtered through celite bed and washed with ethanol (100 ml), and filtrate was concentrated under reduced pressure. The concentrated mass was stirred with THF (100 ml) and n-hexane (100 ml) for 3 h and then filtered to get desired compound 3 as white solid which was used in the next step without further purification. Yield: 67% (7.0 g, 0.096 mol)

Step III: To the stirred suspension of LAH (2.0 g, 0.050 mol, 2.0 eq.) in THF (50 ml) was added portion-wise compound 3 (5 g, 0.025 mol, 1.0 eq.) at 0° C. After the complete addition, the reaction mixture was warmed to RT and then refluxed for 24 h. The reaction mixture was cooled to 0° C. and quenched with 15% NaOH solution (2.5 ml). The reaction mixture was filtered and washed with 10% MeOH/DCM (50 ml). The filtrate was concentrated under reduced pressure to afford crude compound 4 as sticky material. Yield: 100% (4.7 g, 0.025 mol).

Step IV: To the mixture of compound 4 (5.0 g, 0.027 mol, 1.0 eq.) and K₂CO₃ (4.2 g, 0.081 mol, 3.0 eq.) in water (100 ml) was added a solution of Cbz-Cl (05.6 g, 0.032 mol, 1.2 eq.) in toluene (100 ml) at 0° C. and then the reaction mixture was stirred at RT for 16 h. The reaction mixture was diluted with water (100 ml) and layers are separated. Organic layer was dried over sodium sulfate and concentrated to get crude product which was purified by column chromatography (neutral alumina; 1% MeOH/DCM) to yield compound 5. Yield: 46% (4.0 g, 0.0125 mol).

Step V: A solution of compound 5 (4.0 g, 0.125 mol, 1.0 eq.) in THF (100 ml) was added drop-wise to the suspension of LAH (1.9 g, 0.050 mmol, 4.0 eq.) in THF (10 ml) at 0° C. After complete addition, the reaction mixture was warmed to RT and then refluxed for 30 min. The reaction mixture was cooled to 0° C. and quenched with 10% NaOH solution (2 ml). The reaction mixture was filtered and washed with THF (2×100 ml), filtrate was concentrated under reduced pressure to afford desired amine as colorless sticky liquid. Yield: 92% (2.30 g, 0.00116 mol)

AMN-07 was synthesized as mixture of 4 isomers (2cis+2 trans).

Synthesis of: Tert-butyl methyl((1s,3s)-3-(4-methylpiperazin-1-yl)cyclobutyl)carbamate (CC_AMN-11)

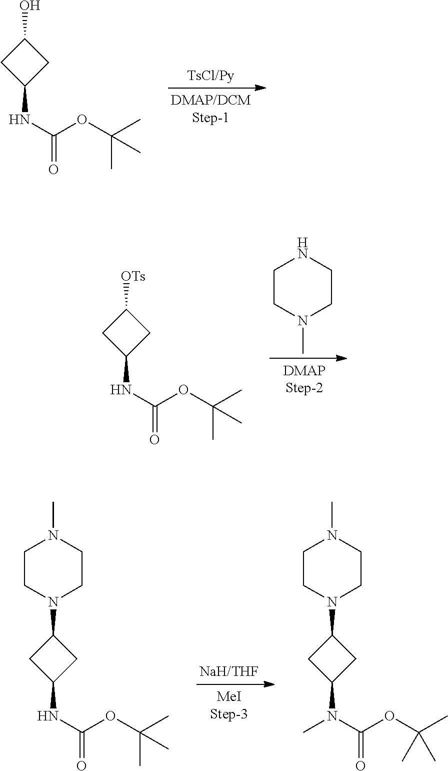

Step 1: To a solution of (Trans)Tert-butyl-3-hydroxycyclobutyl carbamate (1.5 g, 0.008 mol) in DCM (25 ml) are added pyridine (2.48 ml, 0.047 mol), tosyl chloride (1.67 g, 0.0088 mol) and DMAP (0.978 g, 0.008 mol) at 0° C. Then the reaction mixture was stirred at RT for 16 h. The reaction mixture was diluted with diethyl ether (100 ml) and washed with 20% aq. citric acid solution (5×50 ml). Then the organic layer was washed with water (2×50 ml) and brine (50 ml) successively. The organic layer was dried over anh. sodium sulfate and concentrated under reduced pressure. The crude product was purified by column chromatography (silica gel) using ethyl acetate/hexane as eluent. Yield: 62.2% (1.7 g, 0.0049 mol)

Step 2: A mixture of step 1 product (1.7 g, 0.0049 mol), N-methyl piperazine (3.83 g, 0.0383 mol) and DMAP (0.0041 g, 0.0003 mol) was stirred at 100° C. for 2 h. The reaction mixture was cooled to RT and directly was purified by column chromatography (silica gel) using methanol/dichloromethane as eluent to give the pure desired product as white solid. Yield: 74.6% (1 g, 0.0037 mol)

Step 3: To a suspension of sodium hydride (60% in oil, 0.187 g, 0.0046 mol) solution in THF (20 ml) was added slowly a solution of step 2 product (0.7 g, 0.0026 mol) in THF (10 ml) at 0° C. The reaction mixture was stirred at RT for 30 min. Then the reaction mixture was cooled to 0° C. and iodomethane (0.335 ml, 0.0052 mol) was added at the same temperature drop wise. The reaction mixture was stirred at RT for another 2 h. The reaction was quenched and diluted with ice-water (50 ml). The reaction mixture was extracted with 30% isopropanol in dichloromethane (3×50 ml) and washed with water (50 ml) and brine (50 ml) successively. The organic layer was dried over anh. sodium sulfate and concentrated under reduced pressure. The crude product was purified by column chromatography (silica gel) using methanol/dichloromethane as eluent. Yield: 67.9% (0.5 g, 0.0017 mol)

### 3) Library Synthesis

### General:

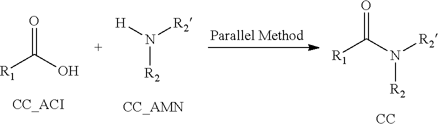

The amines CC_AMN obtained are reacted in parallel according to the Parallel Method with acids CC_ACI to give the amidic products CC. The correlation of products (CC) to the units used (CC_ACI) can be seen from the synthesis matrix.

The crude products of the parallel synthesis are purified by column chromatography. It is possible to demonstrate the identity of the products by analytical HPLC-MS measurements.

### Optional Parallel Method: Amide Formation

HATU (2 eq.) is added to a methylene chloride solution (3 ml/mmol) of the acid unit CC_ACI (1 eq.) at 0° C. and the mixture is stirred for 15 min. In a further round-bottomed flask, a methylene chloride solution (1 ml/mmol) of the Boc-deprotected amine unit CC_AMN (1.5 eq.) is cooled in an ice bath, DIPEA (3 eq.) is added and the mixture is then added to the acid unit at 0° C. The reaction mixture is stirred at room temperature for 16 h and finally diluted with methylene chloride. The organic phase is washed successively with aqueous NH₄Cl solution, NaHCO₃ solution and sat. NaCl solution, dried over sodium sulfate and concentrated under reduced pressure. The crude product is purified via a Biotage parallel purification system. Some compounds are purified manually by column chromatography over neutral aluminium oxide with methanol/methylene chloride as the mobile phase. A few compounds are purified via prep. HPLC.

### Parallel Method:

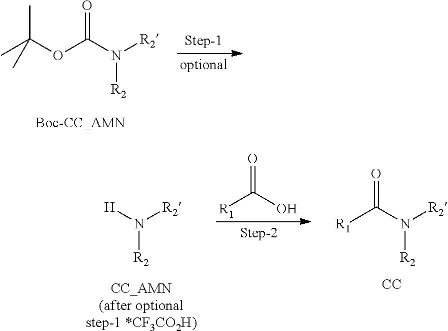

Step 1: Boc-protected amine BB (1 eqv) was treated with 20% TFA in DCM (10 mL/mol) at 0° C. and the resulting reaction mixture was allowed to stir at 25° C. for 4 hrs (monitored by TLC). Solvent was completely evaporated, dried properly to remove traces of TFA and the residue was directly used in library synthesis.

Step 2: To a dichloromethane solution (3 mL/mmol) of acid BBs (1 eqv) was added HATU (2 eqv) at 0° C. and reaction mixture was stirred at the same temperature for another 15 mins. In another R.B flask, Boc-deprotected amine BB (1.5 eqv) in dichloromethane (1 mL/mmol) was cooled in ice bath, treated with DIPEA (3 eqv) and it was added to the reaction mixture at 0° C. Reaction mixture was stirred at RT for 16 hrs and diluted with dichloromethane. Organic layer was successively ished with aqueous ammonium chloride, sodium bicarbonate and brine and finally dried over sodium sulfate. Evaporation of organic layer under reduced pressure gave the crude product, which was purified by prep. HPLC purification using aqueous ammonia method.

### Synthesis Matrix:

| Example no. | Name | Acid (CC_ACI) | Amine (CC_AMN) |
|---|---|---|---|
| CC-01 | 2-[[2-[(4-Methoxy-2,6-dimethyl- | 2-[[2-[(4-Methoxy-2,6-dimethyl- | Methyl-[cis-3-(4-methyl- |
| | phenyl)sulfonyl]-1,2,3,4-tetrahydro- | phenyl)sulfonyl]-1,2,3,4-tetrahydro- | piperazin-1-yl)-cyclohexyl]- |
| | pyrrolo[1,2-a]pyrazin-1-yl]-methoxy]-N- | pyrrolo[1,2-a]pyrazin-1-yl]- | amine (CC_AMN-01) |
| | methyl-N-[cis-3-(4-methyl-piperazin-1-yl)- | methoxy]-acetic acid (CC_ACI-01) | |
| | cyclohexyl]-acetamide | | |
| CC-02 | 4-[2-[(4-Methoxy-2,6-dimethyl- | 4-[2-[(4-Methoxy-2,6-dimethyl- | Methyl-[cis-3-(4-methyl- |
| | phenyl)sulfonyl]-1,2,3,4-tetrahydro- | phenyl)sulfonyl]-1,2,3,4-tetrahydro- | piperazin-1-yl)-cyclohexyl]- |
| | pyrrolo[1,2-a]pyrazin-1-yl]-N-methyl-N-[cis-3- | pyrrolo[1,2-a]pyrazin-1-yl]-butyric | amine (CC_AMN-01) |
| | (4-methyl-piperazin-1-yl)-cyclohexyl]- | acid (CC_ACI-02) | |
| | butyramide | | |
| CC-03 | 2-[[2-[(2,6-Dichloro-3-methyl- | 2-[[2-[(2,6-Dichloro-3-methyl- | Methyl-[cis-3-(4-methyl- |
| | phenyl)sulfonyl]-1,2,3,4-tetrahydro- | phenyl)sulfonyl]-1,2,3,4-tetrahydro- | piperazin-1-yl)-cyclohexyl]- |
| | pyrrolo[1,2-a]pyrazin-1-yl]-methoxy]-N- | pyrrolo[1,2-a]pyrazin-1-yl]- | amine (CC_AMN-01) |
| | methyl-N-[cis-3-(4-methyl-piperazin-1-yl)- | methoxy]-acetic acid (CC_ACI-03) | |
| | cyclohexyl]-acetamide | | |
| CC-04 | 2-[2-[(6-Methoxy-naphthalen-2-yl)sulfonyl]- | 2-[2-[(6-Methoxy-naphthalen-2- | Methyl-[cis-3-(4-methyl- |
| | 1,2,3,4-tetrahydro-pyrrolo[1,2-a]pyrazin-1-yl]- | yl)sulfonyl]-1,2,3,4-tetrahydro- | piperazin-1-yl)-cyclohexyl]- |
| | N-methyl-N-[cis-3-(4-methyl-piperazin-1-yl)- | pyrrolo[1,2-a]pyrazin-1-yl]-acetic | amine (CC_AMN-01) |
| | cyclohexyl]-acetamide | acid (CC_ACI-04) | |
| CC-05 | N-Methyl-N-[cis-3-(4-methyl-piperazin-1-yl)- | 3-[2-[[3- | Methyl-[cis-3-(4-methyl- |
| | cyclohexyl]-3-[2-[[3- | (Trifluoromethyl)phenyl]sulfonyl]- | piperazin-1-yl)-cyclohexyl]- |
| | (trifluoromethyl)phenyl]sulfonyl]-1,2,3,4- | 1,2,3,4-tetrahydro-pyrrolo[1,2- | amine (CC_AMN-01) |
| | tetrahydro-pyrrolo[1,2-a]pyrazin-1-yl]- | a]pyrazin-1-yl]-propionic acid | |
| | propionamide | (CC_ACI-05) | |
| CC-06 | 2-[[2-[(4-Methoxy-2,6-dimethyl- | 2-[[2-[(4-Methoxy-2,6-dimethyl- | [cis-3-(4-Methyl-piperazin-1-yl)- |
| | phenyl)sulfonyl]-1,2,3,4-tetrahydro- | phenyl)sulfonyl]-1,2,3,4-tetrahydro- | cyclohexyl]-amine (CC_AMN-02) |
| | pyrrolo[1,2-a]pyrazin-1-yl]-methoxy]-N-[cis-3- | pyrrolo[1,2-a]pyrazin-1-yl]- | |
| | (4-methyl-piperazin-1-yl)-cyclohexyl]- | methoxy]-acetic acid (CC_ACI-01) | |
| | acetamide | | |
| CC-07 | 4-[2-[(4-Methoxy-2,6-dimethyl- | 4-[2-[(4-Methoxy-2,6-dimethyl- | [cis-3-(4-Methyl-piperazin-1-yl)- |
| | phenyl)sulfonyl]-1,2,3,4-tetrahydro- | phenyl)sulfonyl]-1,2,3,4-tetrahydro- | cyclohexyl]-amine (CC_AMN-02) |
| | pyrrolo[1,2-a]pyrazin-1-yl]-N-[cis-3-(4-methyl- | pyrrolo[1,2-a]pyrazin-1-yl]-butyric | |
| | piperazin-1-yl)-cyclohexyl]-butyramide | acid (CC_ACI-02) | |
| CC-08 | 2-[[2-[(2,6-Dichloro-3-methyl- | 2-[[2-[(2,6-Dichloro-3-methyl- | [cis-3-(4-Methyl-piperazin-1-yl)- |
| | phenyl)sulfonyl]-1,2,3,4-tetrahydro- | phenyl)sulfonyl]-1,2,3,4-tetrahydro- | cyclohexyl]-amine (CC_AMN-02) |
| | pyrrolo[1,2-a]pyrazin-1-yl]-methoxy]-N-[cis-3- | pyrrolo[1,2-a]pyrazin-1-yl]- | |
| | (4-methyl-piperazin-1-yl)-cyclohexyl]- | methoxy]-acetic acid (CC_ACI-03) | |
| | acetamide | | |
| CC-09 | 2-[2-[(6-Methoxy-naphthalen-2-yl)sulfonyl]- | 2-[2-[(6-Methoxy-naphthalen-2- | [cis-3-(4-Methyl-piperazin-1-yl)- |
| | 1,2,3,4-tetrahydro-pyrrolo[1,2-a]pyrazin-1-yl]- | yl)sulfonyl]-1,2,3,4-tetrahydro- | cyclohexyl]-amine (CC_AMN-02) |
| | N-[cis-3-(4-methyl-piperazin-1-yl)- | pyrrolo[1,2-a]pyrazin-1-yl]-acetic | |
| | cyclohexyl]-acetamide | acid (CC_ACI-04) | |
| CC-10 | N-[cis-3-(4-Methyl-piperazin-1-yl)- | 3-[2-[[3- | [cis-3-(4-Methyl-piperazin-1-yl)- |
| | cyclohexyl]-3-[2-[[3- | (Trifluoromethyl)phenyl]sulfonyl]- | cyclohexyl]-amine (CC_AMN-02) |
| | (trifluoromethyl)phenyl]sulfonyl]-1,2,3,4- | 1,2,3,4-tetrahydro-pyrrolo[1,2- | |
| | tetrahydro-pyrrolo[1,2-a]pyrazin-1-yl]- | a]pyrazin-1-yl]-propionic acid | |
| | propionamide | (CC_ACI-05) | |
| CC-11 | 2-[[2-[(4-Methoxy-2,6-dimethyl- | 2-[[2-[(4-Methoxy-2,6-dimethyl- | Methyl-[trans-3-(4-methyl- |
| | phenyl)sulfonyl]-1,2,3,4-tetrahydro- | phenyl)sulfonyl]-1,2,3,4-tetrahydro- | piperazin-1-yl)-cyclohexyl]- |
| | pyrrolo[1,2-a]pyrazin-1-yl]-methoxy]-N- | pyrrolo[1,2-a]pyrazin-1-yl]- | amine (CC_AMN-04) |
| | methyl-N-[trans-3-(4-methyl-piperazin-1-yl)- | methoxy]-acetic acid (CC_ACI-01) | |
| | cyclohexyl]-acetamide | | |
| CC-12 | 4-[2-[(4-Methoxy-2,6-dimethyl- | 4-[2-[(4-Methoxy-2,6-dimethyl- | Methyl-[trans-3-(4-methyl- |
| | phenyl)sulfonyl]-1,2,3,4-tetrahydro- | phenyl)sulfonyl]-1,2,3,4-tetrahydro- | piperazin-1-yl)-cyclohexyl]- |
| | pyrrolo[1,2-a]pyrazin-1-yl]-N-methyl-N-[trans- | pyrrolo[1,2-a]pyrazin-1-yl]-butyric | amine (CC_AMN-04) |
| | 3-(4-methyl-piperazin-1-yl)-cyclohexyl]- | acid (CC_ACI-02) | |
| | butyramide | | |
| CC-13 | 2-[[2-[(2,6-Dichloro-3-methyl- | 2-[[2-[(2,6-Dichloro-3-methyl- | Methyl-[trans-3-(4-methyl- |
| | phenyl)sulfonyl]-1,2,3,4-tetrahydro- | phenyl)sulfonyl]-1,2,3,4-tetrahydro- | piperazin-1-yl)-cyclohexyl]- |
| | pyrrolo[1,2-a]pyrazin-1-yl]-methoxy]-N- | pyrrolo[1,2-a]pyrazin-1-yl]- | amine (CC_AMN-04) |
| | methyl-N-[trans-3-(4-methyl-piperazin-1-yl)- | methoxy]-acetic acid (CC_ACI-03) | |
| | cyclohexyl]-acetamide | | |
| CC-14 | 2-[2-[(6-Methoxy-naphthalen-2-yl)sulfonyl]- | 2-[2-[(6-Methoxy-naphthalen-2- | Methyl-[trans-3-(4-methyl- |
| | 1,2,3,4-tetrahydro-pyrrolo[1,2-a]pyrazin-1-yl]- | yl)sulfonyl]-1,2,3,4-tetrahydro- | piperazin-1-yl)-cyclohexyl]- |
| | N-methyl-N-[trans-3-(4-methyl-piperazin-1- | pyrrolo[1,2-a]pyrazin-1-yl]-acetic | amine (CC_AMN-04) |
| | yl)-cyclohexyl]-acetamide | acid (CC_ACI-04) | |
| CC-15 | N-Methyl-N-[trans-3-(4-methyl-piperazin-1- | 3-[2-[[3- | Methyl-[trans-3-(4-methyl- |
| | yl)-cyclohexyl]-3-[2-[[3- | (Trifluoromethyl)phenyl]sulfonyl]- | piperazin-1-yl)-cyclohexyl]- |
| | (trifluoromethyl)phenyl]sulfonyl]-1,2,3,4- | 1,2,3,4-tetrahydro-pyrrolo[1,2- | amine (CC_AMN-04) |
| | tetrahydro-pyrrolo[1,2-a]pyrazin-1-yl]- | a]pyrazin-1-yl]-propionic acid | |
| | propionamide | (CC_ACI-05) | |
| CC-16 | N-[cis-3-(4-Isopropyl-piperazin-1-yl)- | 2-[[2-[(4-Methoxy-2,6-dimethyl- | [cis-3-(4-Isopropyl-piperazin-1- |
| | cyclohexyl]-2-[[2-[(4-methoxy-2,6-dimethyl- | phenyl)sulfonyl]-1,2,3,4-tetrahydro- | yl)-cyclohexyl]-methyl-amine |
| | phenyl)sulfonyl]-1,2,3,4-tetrahydro- | pyrrolo[1,2-a]pyrazin-1-yl]- | (CC_AMN-05) |
| | pyrrolo[1,2-a]pyrazin-1-yl]-methoxy]-N- | methoxy]-acetic acid (CC_ACI-01) | |
| | methyl-acetamide | | |
| CC-17 | N-[cis-3-(4-Isopropyl-piperazin-1-yl)- | 4-[2-[(4-Methoxy-2,6-dimethyl- | [cis-3-(4-Isopropyl-piperazin-1- |
| | cyclohexyl]-4-[2-[(4-methoxy-2,6-dimethyl- | phenyl)sulfonyl]-1,2,3,4-tetrahydro- | yl)-cyclohexyl]-methyl-amine |
| | phenyl)sulfonyl]-1,2,3,4-tetrahydro- | pyrrolo[1,2-a]pyrazin-1-yl]-butyric | (CC_AMN-05) |
| | pyrrolo[1,2-a]pyrazin-1-yl]-N-methyl- | acid (CC_ACI-02) | |
| | butyramide | | |
| CC-18 | 2-[[2-[(2,6-Dichloro-3-methyl- | 2-[[2-[(2,6-Dichloro-3-methyl- | [cis-3-(4-Isopropyl-piperazin-1- |
| | phenyl)sulfonyl]-1,2,3,4-tetrahydro- | phenyl)sulfonyl]-1,2,3,4-tetrahydro- | yl)-cyclohexyl]-methyl-amine |
| | pyrrolo[1,2-a]pyrazin-1-yl]-methoxy]-N-[cis-3- | pyrrolo[1,2-a]pyrazin-1-yl]- | (CC_AMN-05) |
| | (4-isopropyl-piperazin-1-yl)-cyclohexyl]-N- | methoxy]-acetic acid (CC_ACI-03) | |
| | methyl-acetamide | | |
| CC-19 | N-[cis-3-(4-Isopropyl-piperazin-1-yl)- | 2-[2-[(6-Methoxy-naphthalen-2- | [cis-3-(4-Isopropyl-piperazin-1- |
| | cyclohexyl]-2-[2-[(6-methoxy-naphthalen-2- | yl)sulfonyl]-1,2,3,4-tetrahydro- | yl)-cyclohexyl]-methyl-amine |
| | yl)sulfonyl]-1,2,3,4-tetrahydro-pyrrolo[1,2- | pyrrolo[1,2-a]pyrazin-1-yl]-acetic | (CC_AMN-05) |
| | a]pyrazin-1-yl]-N-methyl-acetamide | acid (CC_ACI-04) | |
| CC-20 | N-[cis-3-(4-Isopropyl-piperazin-1-yl)- | 3-[2-[[3- | [cis-3-(4-Isopropyl-piperazin-1- |
| | cyclohexyl]-N-methyl-3-[2-[[3- | (Trifluoromethyl)phenyl]sulfonyl]- | yl)-cyclohexyl]-methyl-amine |
| | (trifluoromethyl)phenyl]sulfonyl]-1,2,3,4- | 1,2,3,4-tetrahydro-pyrrolo[1,2- | (CC_AMN-05) |
| | tetrahydro-pyrrolo[1,2-a]pyrazin-1-yl]- | a]pyrazin-1-yl]-propionic acid | |
| | propionamide | (CC_ACI-05) | |
| CC-21 | N-[cis-3-(4-Dimethylamino-piperidin-1-yl)- | 2-[[2-[(4-Methoxy-2,6-dimethyl- | Dimethyl-[1-[cis-3- |
| | cyclohexyl]-2-[[2-[(4-methoxy-2,6-dimethyl- | phenyl)sulfonyl]-1,2,3,4-tetrahydro- | methylamino-cyclohexyl]- |
| | phenyl)sulfonyl]-1,2,3,4-tetrahydro- | pyrrolo[1,2-a]pyrazin-1-yl]- | piperidin-4-yl]-amine |
| | pyrrolo[1,2-a]pyrazin-1-yl]-methoxy]-N- | methoxy]-acetic acid (CC_ACI-01) | (CC_AMN-06) |
| | methyl-acetamide | | |
| CC-22 | N-[cis-3-(4-Dimethylamino-piperidin-1-yl)- | 4-[2-[(4-Methoxy-2,6-dimethyl- | Dimethyl-[1-[cis-3- |
| | cyclohexyl]-4-[2-[(4-methoxy-2,6-dimethyl- | phenyl)sulfonyl]-1,2,3,4-tetrahydro- | methylamino-cyclohexyl]- |
| | phenyl)sulfonyl]-1,2,3,4-tetrahydro- | pyrrolo[1,2-a]pyrazin-1-yl]-butyric | piperidin-4-yl]-amine |
| | pyrrolo[1,2-a]pyrazin-1-yl]-N-methyl- | acid (CC_ACI-02) | (CC_AMN-06) |
| | butyramide | | |
| CC-23 | 2-[[2-[(2,6-Dichloro-3-methyl- | 2-[[2-[(2,6-Dichloro-3-methyl- | Dimethyl-[1-[cis-3- |
| | phenyl)sulfonyl]-1,2,3,4-tetrahydro- | phenyl)sulfonyl]-1,2,3,4-tetrahydro- | methylamino-cyclohexyl]- |
| | pyrrolo[1,2-a]pyrazin-1-yl]-methoxy]-N-[cis-3- | pyrrolo[1,2-a]pyrazin-1-yl]- | piperidin-4-yl]-amine |
| | (4-dimethylamino-piperidin-1-yl)-cyclohexyl]- | methoxy]-acetic acid (CC_ACI-03) | (CC_AMN-06) |
| | N-methyl-acetamide | | |
| CC-24 | N-[cis-3-(4-Dimethylamino-piperidin-1-yl)- | 2-[2-[(6-Methoxy-naphthalen-2- | Dimethyl-[1-[cis-3- |
| | cyclohexyl]-2-[2-[(6-methoxy-naphthalen-2- | yl)sulfonyl]-1,2,3,4-tetrahydro- | methylamino-cyclohexyl]- |
| | yl)sulfonyl]-1,2,3,4-tetrahydro-pyrrolo[1,2- | pyrrolo[1,2-a]pyrazin-1-yl]-acetic | piperidin-4-yl]-amine |
| | alpyrazin-1-yl]-N-methyl-acetamide | acid (CC_ACI-04) | (CC_AMN-06) |
| CC-25 | N-[cis-3-(4-Dimethylamino-piperidin-1-yl)- | 3-[2-[[3- | Dimethyl-[1-[cis-3- |
| | cyclohexyl]-N-methyl-3-[2-[[3- | (Trifluoromethyl)phenyl]sulfonyl]- | methylamino-cyclohexyl]- |
| | (trifluoromethyl)phenyl]sulfonyl]-1,2,3,4- | 1,2,3,4-tetrahydro-pyrrolo[1,2- | piperidin-4-yl]-amine |
| | tetrahydro-pyrrolo[1,2-a]pyrazin-1-yl]- | a]pyrazin-1-yl]-propionic acid | (CC_AMN-06) |
| | propionamide | (CC_ACI-05) | |
| CC-26 | 2-[[2-[(4-Methoxy-2,6-dimethyl- | 2-[[2-[(4-Methoxy-2,6-dimethyl- | Methyl-[cis-3-morpholin-4-yl- |
| | phenyl)sulfonyl]-1,2,3,4-tetrahydro- | phenyl)sulfonyl]-1,2,3,4-tetrahydro- | cyclohexyl]-amine (CC_AMN-07) |
| | pyrrolo[1,2-a]pyrazin-1-yl]-methoxy]-N- | pyrrolo[1,2-a]pyrazin-1-yl]- | |
| | methyl-N-[cis-3-morpholin-4-yl-cyclohexyl]- | methoxy]-acetic acid (CC_ACI-01) | |
| | acetamide | | |
| CC-27 | 4-[2-[(4-Methoxy-2,6-dimethyl- | 4-[2-[(4-Methoxy-2,6-dimethyl- | Methyl-[cis-3-morpholin-4-yl- |
| | phenyl)sulfonyl]-1,2,3,4-tetrahydro- | phenyl)sulfonyl]-1,2,3,4-tetrahydro- | cyclohexyl]-amine (CC_AMN-07) |
| | pyrrolo[1,2-a]pyrazin-1-yl]-N-methyl-N-[cis-3- | pyrrolo[1,2-a]pyrazin-1-yl]-butyric | |
| | morpholin-4-yl-cyclohexyl]-butyramide | acid (CC_ACI-02) | |
| CC-28 | 2-[[2-[(2,6-Dichloro-3-methyl- | 2-[[2-[(2,6-Dichloro-3-methyl- | Methyl-[cis-3-morpholin-4-yl- |
| | phenyl)sulfonyl]-1,2,3,4-tetrahydro- | phenyl)sulfonyl]-1,2,3,4-tetrahydro- | cyclohexyl]-amine (CC_AMN-07) |
| | pyrrolo[1,2-a]pyrazin-1-yl]-methoxy]-N- | pyrrolo[1,2-a]pyrazin-1-yl]- | |
| | methyl-N-[cis-3-morpholin-4-yl-cyclohexyl]- | methoxy]-acetic acid (CC_ACI-03) | |
| | acetamide | | |
| CC-29 | 2-[2-[(6-Methoxy-naphthalen-2-yl)sulfonyl]- | 2-[2-[(6-Methoxy-naphthalen-2- | Methyl-[cis-3-morpholin-4-yl- |
| | 1,2,3,4-tetrahydro-pyrrolo[1,2-a]pyrazin-1-yl]- | yl)sulfonyl]-1,2,3,4-tetrahydro- | cyclohexyl]-amine (CC_AMN-07) |
| | N-methyl-N-[cis-3-morpholin-4-yl-cyclohexyl]- | pyrrolo[1,2-a]pyrazin-1-yl]-acetic | |
| | acetamide | acid (CC_ACI-04) | |
| CC-30 | N-Methyl-N-[cis-3-morpholin-4-yl-cyclohexyl]- | 3-[2-[[3- | Methyl-[cis-3-morpholin-4-yl- |
| | 3-[2-[[3-(trifluoromethyl)phenyl]sulfonyl]- | (Trifluoromethyl)phenyl]sulfonyl]- | cyclohexyl]-amine (CC_AMN-07) |
| | 1,2,3,4-tetrahydro-pyrrolo[1,2-a]pyrazin-1-yl]- | 1,2,3,4-tetrahydro-pyrrolo[1,2- | |
| | propionamide | a]pyrazin-1-yl]-propionic acid | |
| | | (CC_ACI-05) | |
| CC-31 | N-[cis-3-(4,4-Difluoro-piperidin-1-yl)- | 2-[[2-[(4-Methoxy-2,6-dimethyl- | [cis-3-(4,4-Difluoro-piperidin-1- |
| | cyclohexyl]-2-[[2-[(4-methoxy-2,6-dimethyl- | phenyl)sulfonyl]-1,2,3,4-tetrahydro- | yl)-cyclohexyl]-methyl-amine |
| | phenyl)sulfonyl]-1,2,3,4-tetrahydro- | pyrrolo[1,2-a]pyrazin-1-yl]- | (CC_AMN-08) |
| | pyrrolo[1,2-a]pyrazin-1-yl]-methoxy]-N- | methoxy]-acetic acid (CC_ACI-01) | |
| | methyl-acetamide | | |
| CC-32 | N-[cis-3-(4,4-Difluoro-piperidin-1-yl)- | 4-[2-[(4-Methoxy-2,6-dimethyl- | [cis-3-(4,4-Difluoro-piperidin-1- |
| | cyclohexyl]-4-[2-[(4-methoxy-2,6-dimethyl- | phenyl)sulfonyl]-1,2,3,4-tetrahydro- | yl)-cyclohexyl]-methyl-amine |
| | phenyl)sulfonyl]-1,2,3,4-tetrahydro- | pyrrolo[1,2-a]pyrazin-1-yl]-butyric | (CC_AMN-08) |
| | pyrrolo[1,2-a]pyrazin-1-yl]-N-methyl- | acid (CC_ACI-02) | |
| | butyramide | | |
| CC-33 | 2-[[2-[(2,6-Dichloro-3-methyl- | 2-[[2-[(2,6-Dichloro-3-methyl- | [cis-3-(4,4-Difluoro-piperidin-1- |
| | phenyl)sulfonyl]-1,2,3,4-tetrahydro- | phenyl)sulfonyl]-1,2,3,4-tetrahydro- | yl)-cyclohexyl]-methyl-amine |
| | pyrrolo[1,2-a]pyrazin-1-yl]-methoxy]-N-[cis-3- | pyrrolo[1,2-a]pyrazin-1-yl]- | (CC_AMN-08) |
| | (4,4-difluoro-piperidin-1-yl)-cyclohexyl]-N- | methoxy]-acetic acid (CC_ACI-03) | |
| | methyl-acetamide | | |
| CC-34 | N-[cis-3-(4,4-Difluoro-piperidin-1-yl)- | 2-[2-[(6-Methoxy-naphthalen-2- | [cis-3-(4,4-Difluoro-piperidin-1- |
| | cyclohexyl]-2-[2-[(6-methoxy-naphthalen-2- | yl)sulfonyl]-1,2,3,4-tetrahydro- | yl)-cyclohexyl]-methyl-amine |
| | yl)sulfonyl]-1,2,3,4-tetrahydro-pyrrolo[1,2- | pyrrolo[1,2-a]pyrazin-1-yl]-acetic | (CC_AMN-08) |
| | a]pyrazin-1-yl]-N-methyl-acetamide | acid (CC_ACI-04) | |
| CC-35 | N-[cis-3-(4,4-Difluoro-piperidin-1-yl)- | 3-[2-[[3- | [cis-3-(4,4-Difluoro-piperidin-1- |
| | cyclohexyl]-N-methyl-3-[2-[[3- | (Trifluoromethyl)phenyl]sulfonyl]- | yl)-cyclohexyl]-methyl-amine |
| | (trifluoromethyl)phenyl]sulfonyl]-1,2,3,4- | 1,2,3,4-tetrahydro-pyrrolo[1,2- | (CC_AMN-08) |
| | tetrahydro-pyrrolo[1,2-a]pyrazin-1-yl]- | a]pyrazin-1-yl]-propionic acid | |
| | propionamide | (CC_ACI-05) | |
| CC-36 | 2-[[2-[(4-Methoxy-2,6-dimethyl- | 2-[[2-[(4-Methoxy-2,6-dimethyl- | Methyl-[cis-3-(1,2,3,4- |
| | phenyl)sulfonyl]-1,2,3,4-tetrahydro- | phenyl)sulfonyl]-1,2,3,4-tetrahydro- | tetrahydro-[2,6]naphthyridin-2- |
| | pyrrolo[1,2-a]pyrazin-1-yl]-methoxy]-N- | pyrrolo[1,2-a]pyrazin-1-yl]- | yl)-cyclohexyl]-amine |
| | methyl-N-[cis-3-(1,2,3,4-tetrahydro- | methoxy]-acetic acid (CC_ACI-01) | (CC_AMN-09) |
| | [2,6]naphthyridin-2-yl)-cyclohexyl]-acetamide | | |
| CC-37 | 4-[2-[(4-Methoxy-2,6-dimethyl- | 4-[2-[(4-Methoxy-2,6-dimethyl- | Methyl-[cis-3-(1,2,3,4- |
| | phenyl)sulfonyl]-1,2,3,4-tetrahydro- | phenyl)sulfonyl]-1,2,3,4-tetrahydro- | tetrahydro-[2,6]naphthyridin-2- |
| | pyrrolo[1,2-a]pyrazin-1-yl]-N-methyl-N-[cis-3- | pyrrolo[1,2-a]pyrazin-1-yl]-butyric | yl)-cyclohexyl]-amine |
| | (1,2,3,4-tetrahydro-[2,6]naphthyridin-2-yl)- | acid (CC_ACI-02) | (CC_AMN-09) |
| | cyclohexyl]-butyramide | | |
| CC-38 | 2-[[2-[(2,6-Dichloro-3-methyl- | 2-[[2-[(2,6-Dichloro-3-methyl- | Methyl-[cis-3-(1,2,3,4- |
| | phenyl)sulfonyl]-1,2,3,4-tetrahydro- | phenyl)sulfonyl]-1,2,3,4-tetrahydro- | tetrahydro-[2,6]naphthyridin-2- |
| | pyrrolo[1,2-a]pyrazin-1-yl]-methoxy]-N- | pyrrolo[1,2-a]pyrazin-1-yl]- | yl)-cyclohexyl]-amine |
| | methyl-N-[cis-3-(1,2,3,4-tetrahydro- | methoxy]-acetic acid (CC_ACI-03) | (CC_AMN-09) |
| | [2,6]naphthyridin-2-yl)-cyclohexyl]-acetamide | | |
| CC-39 | 2-[2-[(6-Methoxy-naphthalen-2-yl)sulfonyl]- | 2-[2-[(6-Methoxy-naphthalen-2- | Methyl-[cis-3-(1,2,3,4- |
| | 1,2,3,4-tetrahydro-pyrrolo[1,2-a]pyrazin-1-yl]- | yl)sulfonyl]-1,2,3,4-tetrahydro- | tetrahydro-[2,6]naphthyridin-2- |
| | N-methyl-N-[cis-3-(1,2,3,4-tetrahydro- | pyrrolo[1,2-a]pyrazin-1-yl]-acetic | yl)-cyclohexyl]-amine |
| | [2,6]naphthyridin-2-yl)-cyclohexyl]-acetamide | acid (CC_ACI-04) | (CC_AMN-09) |
| CC-40 | N-Methyl-N-[cis-3-(1,2,3,4-tetrahydro- | 3-[2-[[3- | Methyl-[cis-3-(1,2,3,4- |
| | [2,6]naphthyridin-2-yl)-cyclohexyl]-3-[2-[[3- | (Trifluoromethyl)phenyl]sulfonyl]- | tetrahydro-[2,6]naphthyridin-2- |
| | (trifluoromethyl)phenyl]sulfonyl]-1,2,3,4- | 1,2,3,4-tetrahydro-pyrrolo[1,2- | yl)-cyclohexyl]-amine |
| | tetrahydro-pyrrolo[1,2-a]pyrazin-1-yl]- | a]pyrazin-1-yl]-propionic acid | (CC_AMN-09) |
| | propionamide | (CC_ACI-05) | |
| CC-50 | N-[3-(4-Isopropyl-piperazin-1-yl)-cyclohexyl]- | 2-[[2-[(4-Methoxy-2,6-dimethyl- | [3-(4-Isopropyl-piperazin-1-yl)- |
| | 2-[[2-[(4-methoxy-2,6-dimethyl- | phenyl)sulfonyl]-1,2,3,4-tetrahydro- | cyclohexyl]-methyl-amine |
| | phenyl)sulfonyl]-1,2,3,4-tetrahydro- | pyrrolo[1,2-a]pyrazin-1-yl]- | (CC_AMN-05_Mix) |
| | pyrrolo[1,2-a]pyrazin-1-yl]-methoxy]-N- | methoxy]-acetic acid (CC_ACI-01) | |
| | methyl-acetamide | | |
| CC-51 | N-[3-(4-Isopropyl-piperazin-1-yl)-cyclohexyl]- | 4-[2-[(4-Methoxy-2,6-dimethyl- | [3-(4-Isopropyl-piperazin-1-yl)- |
| | 4-[2-[(4-methoxy-2,6-dimethyl- | phenyl)sulfonyl]-1,2,3,4-tetrahydro- | cyclohexyl]-methyl-amine |
| | phenyl)sulfonyl]-1,2,3,4-tetrahydro- | pyrrolo[1,2-a]pyrazin-1-yl]-butyric | (CC_AMN-05_Mix) |
| | pyrrolo[1,2-a]pyrazin-1-yl]-N-methyl- | acid (CC_ACI-02) | |
| | butyramide | | |
| CC-52 | N-[3-(4-Isopropyl-piperazin-1-yl)-cyclohexyl]- | 2-[2-[(6-Methoxy-naphthalen-2- | [3-(4-Isopropyl-piperazin-1-yl)- |
| | 2-[2-[(6-methoxy-naphthalen-2-yl)sulfonyl]- | yl)sulfonyl]-1,2,3,4-tetrahydro- | cyclohexyl]-methyl-amine |
| | 1,2,3,4-tetrahydro-pyrrolo[1,2-a]pyrazin-1-yl]- | pyrrolo[1,2-a]pyrazin-1-yl]-acetic | (CC_AMN-05_Mix) |
| | N-methyl-acetamide | acid (CC_ACI-04) | |
| CC-53 | N-[3-(4-Isopropyl-piperazin-1-yl)-cyclohexyl]- | 3-[2-[[3- | [3-(4-Isopropyl-piperazin-1-yl)- |
| | N-methyl-3-[2-[[3- | (Trifluoromethyl)phenyl]sulfonyl]- | cyclohexyl]-methyl-amine |
| | (trifluoromethyl)phenyl]sulfonyl]-1,2,3,4- | 1,2,3,4-tetrahydro-pyrrolo[1,2- | (CC_AMN-05_Mix) |
| | tetrahydro-pyrrolo[1,2-a]pyrazin-1-yl]- | a]pyrazin-1-yl]-propionic acid | |
| | propionamide | (CC_ACI-05) | |
| CC-54 | 2-[[2-[(4-Methoxy-2,6-dimethyl- | 2-[[2-[(4-Methoxy-2,6-dimethyl- | Methyl-[3-morpholin-4-yl- |
| | phenyl)sulfonyl]-1,2,3,4-tetrahydro- | phenyl)sulfonyl]-1,2,3,4-tetrahydro- | cyclohexyl]-amine (CC_AMN- |
| | pyrrolo[1,2-a]pyrazin-1-yl]-methoxy]-N- | pyrrolo[1,2-a]pyrazin-1-yl]- | 07_Mix) |
| | methyl-N-(3-morpholin-4-yl-cyclohexyl)- | methoxy]-acetic acid (CC_ACI-01) | |
| | acetamide | | |
| CC-55 | 4-[2-[(4-Methoxy-2,6-dimethyl- | 4-[2-[(4-Methoxy-2,6-dimethyl- | Methyl-[3-morpholin-4-yl- |
| | phenyl)sulfonyl]-1,2,3,4-tetrahydro- | phenyl)sulfonyl]-1,2,3,4-tetrahydro- | cyclohexyl]-amine (CC_AMN- |
| | pyrrolo[1,2-a]pyrazin-1-yl]-N-methyl-N-(3- | pyrrolo[1,2-a]pyrazin-1-yl]-butyric | 07_Mix) |
| | morpholin-4-yl-cyclohexyl)-butyramide | acid (CC_ACI-02) | |
| CC-56 | 2-[2-[(6-Methoxy-naphthalen-2-yl)sulfonyl]- | 2-[2-[(6-Methoxy-naphthalen-2- | Methyl-[3-morpholin-4-yl- |
| | 1,2,3,4-tetrahydro-pyrrolo[1,2-a]pyrazin-1-yl]- | yl)suifonyl]-1,2,3,4-tetrahydro- | cyclohexyl]-amine (CC_AMN- |
| | N-methyl-N-(3-morpholin-4-yl-cyclohexyl)- | pyrrolo[1,2-a]pyrazin-1-yl]-acetic | 07_Mix) |
| | acetamide | acid (CC_ACI-04) | |
| CC-57 | N-Methyl-N-(3-morpholin-4-yl-cyclohexyl)-3- | 3-[2-[[3- | Methyl-[3-morpholin-4-yl- |
| | [2-[[3-(trifluoromethyl)phenyl]sulfonyl]- | (Trifluoromethyl)phenyl]sulfonyl]- | cyclohexyl]-amine (CC_AMN- |
| | 1,2,3,4-tetrahydro-pyrrolo[1,2-a]pyrazin-1-yl]- | 1,2,3,4-tetrahydro-pyrrolo[1,2- | 07_Mix) |
| | propionamide | a]pyrazin-1-yl]-propionic acid | |
| | | (CC_ACI-05) | |
| CC-58 | 2-[[2-[(4-Methoxy-2,6-dimethyl- | 2-[[2-[(4-Methoxy-2,6-dimethyl- | Tert-butyl methyl((1s,3s)-3-(4- |
| | phenyl)sulfonyl]-1,2,3,4-tetrahydro- | phenyl)sulfonyl]-1,2,3,4-tetrahydro- | methylpiperazin-1- |
| | pyrrolo[1,2-a]pyrazin-1-yl]-methoxy]-N- | pyrrolo[1,2-a]pyrazin-1-yl]- | yl)cyclobutyl)carbamate |
| | methyl-N-[3-(4-methyl-piperazin-1-yl)- | methoxy]-acetic acid (CC_ACI-01) | (CC_AMN-11) |
| | cyclobutyl]-acetamide | | |
| CC-59 | 4-[2-[(4-Methoxy-2,6-dimethyl- | 4-[2-[(4-Methoxy-2,6-dimethyl- | Tert-butyl methyl((1s,3s)-3-(4- |
| | phenyl)sulfonyl]-1,2,3,4-tetrahydro- | phenyl)sulfonyl]-1,2,3,4-tetrahydro- | methylpiperazin-1- |
| | pyrrolo[1,2-a]pyrazin-1-yl]-N-methyl-N-[3-(4- | pyrrolo[1,2-a]pyrazin-1-yl]-butyric | yl)cyclobutyl)carbamate |
| | methyl-piperazin-1-yl)-cyclobutyl]-butyramide | acid (CC_ACI-02) | (CC_AMN-11) |
| CC-60 | 2-[2-[(6-Methoxy-naphthalen-2-yl)sulfonyl]- | 2-[2-[(6-Methoxy-naphthalen-2- | Tert-butyl methyl((1s,3s)-3-(4- |
| | 1,2,3,4-tetrahydro-pyrrolo[1,2-a]pyrazin-1-yl]- | yl)sulfonyl]-1,2,3,4-tetrahydro- | methylpiperazin-1- |
| | N-methyl-N-[3-(4-methyl-piperazin-1-yl)- | pyrrolo[1,2-a]pyrazin-1-yl]-acetic | yl)cyclobutyl)carbamate |
| | cyclobutyl]-acetamide | acid (CC_ACI-04) | (CC_AMN-11) |
| CC-61 | N-Methyl-N-[3-(4-methyl-piperazin-1-yl)- | 3-[2-[[3- | Tert-butyl methyl((1s,3s)-3-(4- |
| | cyclobutyl]-3-[2-[[3- | (Trifluoromethyl)phenyl]sulfonyl]- | methylpiperazin-1- |
| | (trifluoromethyl)phenyl]sulfonyl]-1,2,3,4- | 1,2,3,4-tetrahydro-pyrrolo[1,2- | yl)cyclobutyl)carbamate |
| | tetrahydro-pyrrolo[1,2-a]pyrazin-1-yl]- | a]pyrazin-1-yl]-propionic acid | (CC_AMN-11) |
| | propionamide | (CC_ACI-05) | |

It is readily apparent to persons skilled in the art that in those cases that the amine building blocks used as a starting material are a mixture of different stereoisomers, the resulting products will also be a mixture of the corresponding stereoisomers.

The respective pure stereoisomer can be can be obtained either by purifying the final compound using well known methods for the isolation of stereoisomers such as column chromatography, if needed on chiral stationary phases or other suitable methods like crystallization. Or by purifying the respective amine building block, utilizing the previously mentioned methods, to obtain the desired stereoisomerically pure building block, which can be utilized to prepare the desired pure final compound.

### Analytical Data

| Example no. | Found M + H |
|---|---|
| CC-01 | Yes |
| CC-02 | Yes |
| CC-04 | Yes |
| CC-05 | Yes |
| CC-06 | Yes |
| CC-07 | Yes |
| CC-09 | Yes |
| CC-10 | Yes |
| CC-50 | Yes |
| CC-51 | Yes |
| CC-52 | Yes |
| CC-53 | Yes |
| CC-54 | Yes |
| CC-55 | Yes |
| CC-56 | Yes |
| CC-57 | Yes |
| CC-58 | Yes |
| CC-59 | Yes |
| CC-60 | Yes |
| CC-61 | Yes |

### Pharmacological Data

The pharmacological data were determined as described above. The following data are given in the following tables by way of example:

| | % Inhibition (rat B1R) | % Inhibition (human B1R) |
|---|---|---|
| Example no. | at 10 μM | at 10 μM |
| SC-01 | 103 | 100 |
| SC-02 | 105 | 100 |
| SC-03 | 61 | 95 |
| SC-05 | 100 | 101 |
| SC-06 | 110 | 97 |

| | % Inhibition (rat B1R) | % Inhibition (human B1R) |
|---|---|---|
| Example no. | at 10 μM | at 10 μM |
| CC-01 | 105 | 100 |
| CC-02 | 104 | 87 |
| CC-04 | 103 | 100 |
| CC-05 | 91 | 43 |
| CC-06 | 100 | 96 |
| CC-07 | 101 | 86 |
| CC-09 | | 100 |
| CC-10 | 36 | 29 |
| CC-50 | | 99 |
| CC-51 | | 99 |
| CC-52 | | 100 |
| CC-53 | | 87 |
| CC-54 | | 98 |
| CC-55 | | 88 |
| CC-56 | | 99 |
| CC-57 | | 39 |
| CC-58 | 88 | 38 |
| CC-59 | 97 | 80 |
| CC-60 | 106 | 99 |
| CC-61 | 14 | 12 |

The foregoing description and examples have been set forth merely to illustrate the invention and are not intended to be limiting. Since modifications of the described embodiments incorporating the spirit and substance of the invention may occur to persons skilled in the art, the invention should be constued broadly to include all variations within the scope of the appended claims and equivalents thereof.

1. A compound corresponding to formula (I): 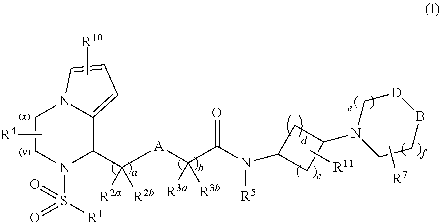
wherein
a represents 0, 1 or 2;
b represents 1 or 2;
A represents C(R^{6a})(R^{6b}), O, or a single bond;
R¹ represents an aryl or heteroaryl group;
R^{2a}, R^{2b}, R^{3a}, R^{3b}, R^{6a} and R^{6b} are each independently selected from the group consisting of H, OH and O—C₁₋₆ alkyl;
R⁴ represents 0 to 4 substituents each independently selected from the group consisting of C₁₋₆ alkyl and C₃₋₈ cycloalkyl; or
R⁴ represents an anellated aryl or heteroaryl ring bonded to the carbon atoms denoted in formula (I) by the letters (x) and (y);
R⁵ represents H; C₁₋₆ alkyl; C₃₋₈ cycloalkyl; or C₃₋₈ cycloalkyl bonded by a C₁₋₆ alkylene group;
R⁷ represents 0 to 4 substituents each independently selected from the group consisting of F; Cl; OH; ═O; C₁₋₆ alkyl; O—C₁₋₆ alkyl; C₃₋₈ cycloalkyl; aryl; heteroaryl; C₃₋₈ cycloalkyl, and aryl or heteroaryl bonded by a C₁₋₆ alkylene group;
c represents 1, 2 or 3;
d represents 1, 2 or 3;
with the proviso that c+d is less than or equal to 4;
e and f each independently represent 0 or 1;
with the proviso that e+f is not 0;
B represents NR⁸, O, CH—N(R^{9a})(R^{9b}), or CF₂;
wherein
R⁸ represents H, C₁₋₆ alkyl or C₃₋₈ cycloalkyl,
R^{9a} and R^{9b} are each independently selected from the group consisting of H, C₁₋₄ alkyl and C₃₋₆ cycloalkyl, or
R^{9a} and R^{9b} together with the N-atom to which they are bonded to form a 4-, 5- or 6-membered heterocycle;
R¹⁰ represents 0 to 2 substituents each independently selected from the group consisting of CH₃, CF₃, F and Cl;
R¹¹ represents 0 to 4 substituents each independently selected from the group consisting of F; Cl; OH; ═O; C₁₋₆ alkyl; O—C₁₋₆ alkyl; C₃₋₈ cycloalkyl; aryl; heteroaryl; C₃₋₈ cycloalkyl, and aryl or heteroaryl bonded by a C₁₋₆ alkylene group;
D represents CH₂;
or
D together with B forms an anellated, 5- or 6-membered heteroaryl or aryl group;
wherein the aforementioned C₁₋₃, C₁₋₄ and C₁₋₆ alkyl, C₁₋₃ and C₁₋₆ alkylene, C₃₋₆-cycloalkyl, C₃₋₈-cycloalkyl, aryl, heteroaryl and heterocyclyl groups may each be unsubstituted or mono- or polysubstituted with identical or different substituents, and the aforementioned C₁₋₃, C₁₋₄ and C₁₋₆ alkyl, C₁₋₃ and C₁₋₆ alkylene groups may each be branched or unbranched;
or a physiologically acceptable salt thereof.

2. A compound according to claim 1, wherein said compound is in the form of an isolated diastereomer or an isolated enantiomer.

3. A compound according to claim 1, wherein said compound is in the form of a mixture of diastereomers or enantiomers in any mixing ratio.

4. A compound according to claim 1, wherein said mixture is a racemic mixture.

5. A compound according to claim 1, wherein
A represents O, and each of R^{2a}, R^{2b}, R^{3a} and R^{3b} represents H;
or
A represents C(R^{6a})(R^{6b}) or a single bond, and R^{2a}, R^{2b}, R^{3a}, R^{3b}, R^{6a} and R^{6b} each independently represent H, F, CF₃, OH, CH₃, O—CH₃ or O—CF₃, with the provisio that not more than two of R^{2a}, R^{2b}, R^{3a}, R^{3b}, R^{6a} and R^{6b} can represent a group other than H at the same time.

6. A compound according to claim 1, wherein:
a represents 0; A represents a single bond, and b represents 1;
or
a and b each represent 1, and A represents a single bond or CR^{6a}R^{6b};
or
a and b each represent 1, and A represents O.

7. A compound according to claim 1, wherein R¹ represents phenyl or naphthyl, each unsubstituted or monosubstituted or identically or differently polysubstituted with substituents selected from the group consisting of —O—C₁₋₃ alkyl, C₁₋₆ alkyl, F, Cl, Br, CF₃ and OCF₃.

8. A compound according to claim 1, corresponding to formula (Ia): 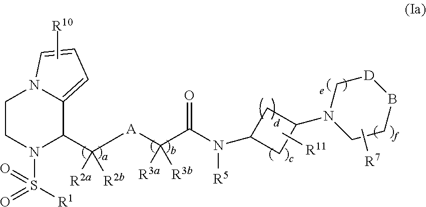
wherein A, B, D, R¹, R^{2a}, R^{2b}, R^{3a}, R^{3b}, R⁵, R⁷, R¹⁰, R¹¹, a, b, c, d, e and f have the respective meanings given in claim 1.

9. A compound according to claim 1, wherein R⁵ represents H; C₁₋₆ alkyl; C₃₋₈ cycloalkyl; or C₃₋₈ cycloalkyl bonded via a C₁₋₃ alkylene group.

10. A compound according to claim 1, wherein:
c represents 1, and d represents 3; or
c represents 3, and d represents 1; or
c and d each represent 2; or
c represents 1, and d represents 2; or
c represents 2, and d represents 1.

11. A compound according to claim 1, wherein R¹¹ represents H.

12. A compound according to claim 1, wherein:
B represents NR⁸, wherein R⁸ is selected from the group consisting of H, C₁₋₄ alkyl and or C₃₋₆ cycloalkyl, and D represents CH₂; or
B represents O, and D represents CH₂; or
B represents CH—N(R^{9a})(R^{9b}), wherein R^{9a} and R^{9b} are each independently selected from the group consisting of H, C₁₋₃ alkyl and C₃₋₄ cycloalkyl, or R^{9a} and R^{9b} together with the N-atom to which they are bonded to form a 4- or 5-membered heterocycle and D represents CH₂; or
B and D together form an anellated pyridinyl moiety.

13. A compound according to claim 1, wherein in formula (I) the substructure represented by formula (II): 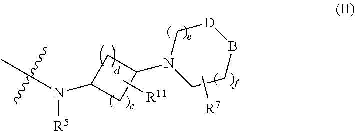
is selected from the group consisting of: 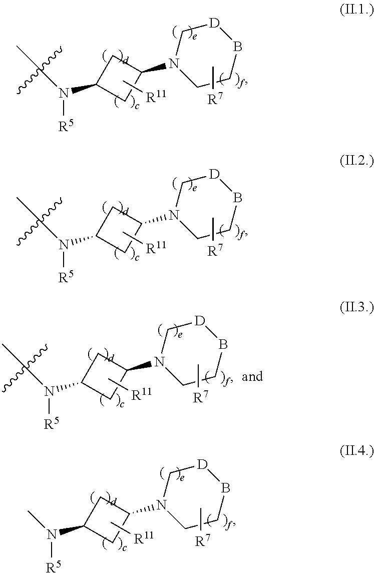
wherein R⁵, R⁷, R¹¹, c, d, e, f, B and D have the respective meanings given in claim 1.

14. A compound according to claim 1, wherein in formula (I) the substructure represented by formula (IIa): 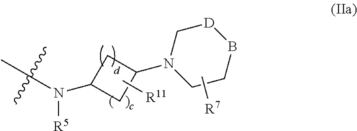
is selected from the group consisting of:

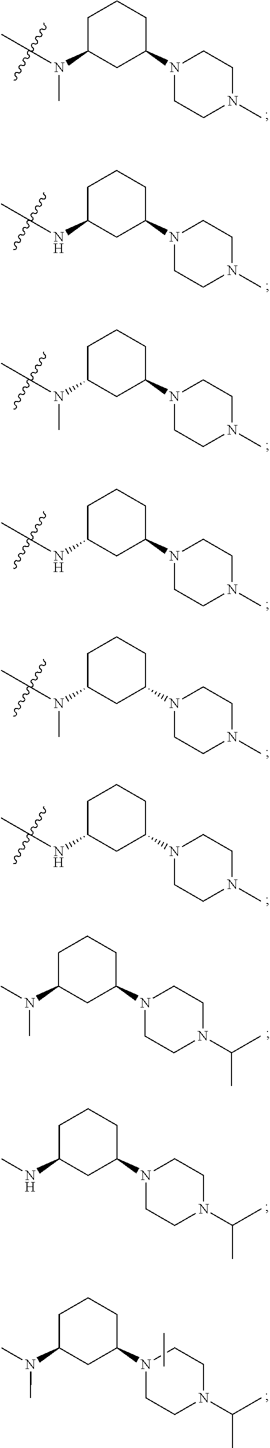

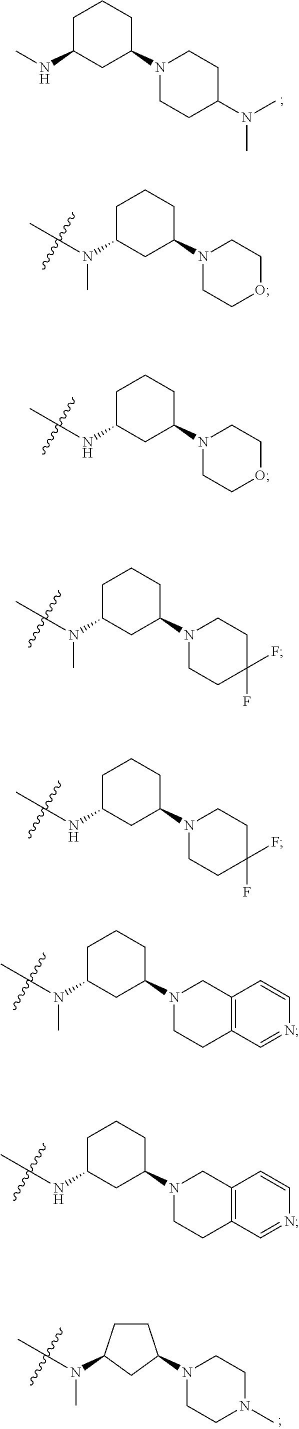

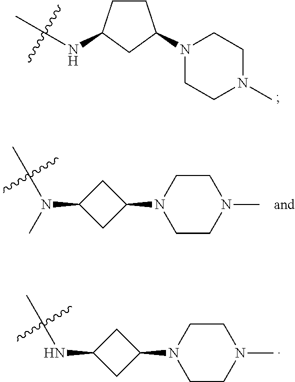

15. A compound according to claim 1, wherein in formula (I) the substructure represented by formula (AC II): 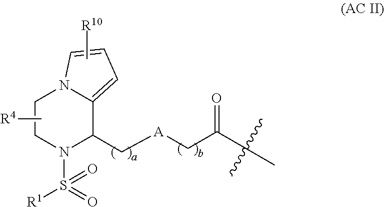
is selected from the group consisting of: 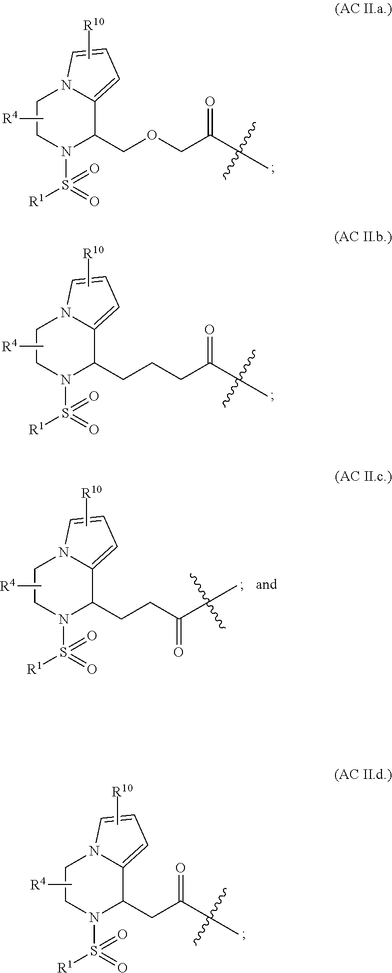
wherein R¹, R⁴ and R¹⁰ have the respective meanings given in claim 1.

16. A compound according to claim 1, wherein R¹ is selected from the group consisting of:
2,6-dimethyl-4-methoxy-phenyl;
2-6-dichloro-3-methylphenyl;
3-trifluoromethyl-phenyl;
2-chloro-6-methyl-phenyl;
4-chloro-2,5-dimethylphenyl, and
6-methoxy-naphthyl.

17. A compound according to claim 1, selected from the group consisting of:
2-[[2-[(4-methoxy-2,6-dimethyl-phenyl)sulfonyl]-1,2,3,4-tetrahydro-pyrrolo[1,2-a]pyrazin-1-yl]-methoxy]-N-methyl-N-[(1S,3R)-3-(4-methyl-piperazin-1-yl)-cyclohexyl]-acetamide,
2-[[2-[(2-chloro-6-methyl-phenyl)sulfonyl]-1,2,3,4-tetrahydro-pyrrolo[1,2-a]pyrazin-1-yl]-methoxy]-N-methyl-N-[(1S,3R)-3-(4-methyl-piperazin-1-yl)-cyclohexyl]-acetamide,
3-[2-[(4-chloro-2,5-dimethyl-phenyl)sulfonyl]-1,2,3,4-tetrahydro-pyrrolo[1,2-a]pyrazin-1-yl]-N-methyl-N-[(1S,3R)-3-(4-methyl-piperazin-1-yl)-cyclohexyl]-propionamide,
2-[[2-[(4-methoxy-2,6-dimethyl-phenyl)sulfonyl]-1,2,3,4-tetrahydro-pyrrolo[1,2-a]pyrazin-1-yl]-methoxy]-N-methyl-N-[4-(4-methyl-piperazin-1-yl)-cyclohexyl]-acetamide,
2-[[2-[(4-methoxy-2,6-dimethyl-phenyl)sulfonyl]-6-methyl-1,2,3,4-tetrahydro-pyrrolo[1,2-a]pyrazin-1-yl]-methoxy]-N-methyl-N-[(1S,3R)-3-(4-methyl-piperazin-1-yl)-cyclohexyl]-acetamide,
2-[[2-[(4-methoxy-2,6-dimethyl-phenyl)sulfonyl]-1,2,3,4-tetrahydro-pyrrolo[1,2-a]pyrazin-1-yl]-methoxy]-N-methyl-N-[cis-3-(4-methyl-piperazin-1-yl)-cyclohexyl]-acetamide,
4-[2-[(4-methoxy-2,6-dimethyl-phenyl)sulfonyl]-1,2,3,4-tetrahydro-pyrrolo[1,2-a]pyrazin-1-yl]-N-methyl-N-[cis-3-(4-methyl-piperazin-1-yl)-cyclohexyl]-butyramide,
2-[[2-[(2,6-dichloro-3-methyl-phenyl)sulfonyl]-1,2,3,4-tetrahydro-pyrrolo[1,2-a]pyrazin-1-yl]-methoxy]-N-methyl-N-[cis-3-(4-methyl-piperazin-1-yl)-cyclohexyl]-acetamide,
2-[2-[(6-methoxy-naphthalen-2-yl)sulfonyl]-1,2,3,4-tetrahydro-pyrrolo[1,2-a]pyrazin-1-yl]-N-methyl-N-[cis-3-(4-methyl-piperazin-1-yl)-cyclohexyl]-acetamide,
N-methyl-N-[cis-3-(4-methyl-piperazin-1-yl)-cyclohexyl]-3-[2-[[3-(trifluoromethyl)phenyl]sulfonyl]-1,2,3,4-tetrahydro-pyrrolo[1,2-a]pyrazin-1-yl]-propionamide,
2-[[2-[(4-methoxy-2,6-dimethyl-phenyl)sulfonyl]-1,2,3,4-tetrahydro-pyrrolo[1,2-a]pyrazin-1-yl]-methoxy]-N-[cis-3-(4-methyl-piperazin-1-yl)-cyclohexyl]-acetamide;
4-[2-[(4-methoxy-2,6-dimethyl-phenyl)sulfonyl]-1,2,3,4-tetrahydro-pyrrolo[1,2-a]pyrazin-1-yl]-N-[cis-3-(4-methyl-piperazin-1-yl)-cyclohexyl]-butyramide;
2-[[2-[(2,6-dichloro-3-methyl-phenyl)sulfonyl]-1,2,3,4-tetrahydro-pyrrolo[1,2-a]pyrazin-1-yl]-methoxy]-N-[cis-3-(4-methyl-piperazin-1-yl)-cyclohexyl]-acetamide;
2-[2-[(6-methoxy-naphthalen-2-yl)sulfonyl]-1,2,3,4-tetrahydro-pyrrolo[1,2-a]pyrazin-1-yl]-N-[cis-3-(4-methyl-piperazin-1-yl)-cyclohexyl]-acetamide;
N-[cis-3-(4-methyl-piperazin-1-yl)-cyclohexyl]-3-[2-[[3-(trifluoromethyl)phenyl]sulfonyl]-1,2,3,4-tetrahydro-pyrrolo[1,2-a]pyrazin-1-yl]-propionamide;
2-[[2-[(4-methoxy-2,6-dimethyl-phenyl)sulfonyl]-1,2,3,4-tetrahydro-pyrrolo[1,2-a]pyrazin-1-yl]-methoxy]-N-methyl-N-[trans-3-(4-methyl-piperazin-1-yl)-cyclohexyl]-acetamide;
4-[2-[(4-methoxy-2,6-dimethyl-phenyl)sulfonyl]-1,2,3,4-tetrahydro-pyrrolo[1,2-a]pyrazin-1-yl]-N-methyl-N-[trans-3-(4-methyl-piperazin-1-yl)-cyclohexyl]-butyramide-2-[[2-[(2,6-dichloro-3-methyl-phenyl)sulfonyl]-1,2,3,4-tetrahydro-pyrrolo[1,2-a]pyrazin-1-yl]-methoxy]-N-methyl-N-[trans-3-(4-methyl-piperazin-1-yl)-cyclohexyl]-acetamide;
2-[2-[(6-methoxy-naphthalen-2-yl)sulfonyl]-1,2,3,4-tetrahydro-pyrrolo[1,2-a]pyrazin-1-yl]-N-methyl-N-[trans-3-(4-methyl-piperazin-1-yl)-cyclohexyl]-acetamide;
N-methyl-N-[trans-3-(4-methyl-piperazin-1-yl)-cyclohexyl]-3-[2-[[3-(trifluoromethyl)phenyl]sulfonyl]-1,2,3,4-tetrahydro-pyrrolo[1,2-a]pyrazin-1-yl]-propionamide;
N-[cis-3-(4-isopropyl-piperazin-1-yl)-cyclohexyl]-2-[[2-[(4-methoxy-2,6-dimethyl-phenyl)sulfonyl]-1,2,3,4-tetrahydro-pyrrolo[1,2-a]pyrazin-1-yl]-methoxy]-N-methyl-acetamide;
N-[cis-3-(4-isopropyl-piperazin-1-yl)-cyclohexyl]-4-[2-[(4-methoxy-2,6-dimethyl-phenyl)sulfonyl]-1,2,3,4-tetrahydro-pyrrolo[1,2-a]pyrazin-1-yl]-N-methyl-butyramide
2-[[2-[(2,6-dichloro-3-methyl-phenyl)sulfonyl]-1,2,3,4-tetrahydro-pyrrolo[1,2-a]pyrazin-1-yl]-methoxy]-N-[cis-3-(4-isopropyl-piperazin-1-yl)-cyclohexyl]-N-methyl-acetamide;
N-[cis-3-(4-isopropyl-piperazin-1-yl)-cyclohexyl]-2-[2-[(6-methoxy-naphthalen-2-yl)sulfonyl]-1,2,3,4-tetrahydro-pyrrolo[1,2-a]pyrazin-1-yl]-N-methyl-acetamide;
N-[cis-3-(4-isopropyl-piperazin-1-yl)-cyclohexyl]-N-methyl-3-[2-[[3-(trifluoromethyl)phenyl]sulfonyl]-1,2,3,4-tetrahydro-pyrrolo[1,2-a]pyrazin-1-yl]-propionamide;
N-[cis-3-(4-dimethylamino-piperidin-1-yl)-cyclohexyl]-2-[[2-[(4-methoxy-2,6-dimethylphenyl)sulfonyl]-1,2,3,4-tetrahydro-pyrrolo[1,2-a]pyrazin-1-yl]-methoxy]-N-methyl-acetamide;
N-[cis-3-(4-dimethylamino-piperidin-1-yl)-cyclohexyl]-4-[2-[(4-methoxy-2,6-dimethylphenyl)sulfonyl]-1,2,3,4-tetrahydro-pyrrolo[1,2-a]pyrazin-1-yl]-N-methyl-butyramide
2-[[2-[(2,6-dichloro-3-methyl-phenyl)sulfonyl]-1,2,3,4-tetrahydro-pyrrolo[1,2-a]pyrazin-1-yl]-methoxy]-N-[cis-3-(4-dimethylamino-piperidin-1-yl)-cyclohexyl]-N-methyl-acetamide;
N-[cis-3-(4-dimethylamino-piperidin-1-yl)-cyclohexyl]-2-[2-[(6-methoxy-naphthalen-2-yl)sulfonyl]-1,2,3,4-tetrahydro-pyrrolo[1,2-a]pyrazin-1-yl]-N-methyl-acetamide;
N-[cis-3-(4-dimethylamino-piperidin-1-yl)-cyclohexyl]-N-methyl-3-[2-[[3-(trifluoromethyl)phenyl]sulfonyl]-1,2,3,4-tetrahydro-pyrrolo[1,2-a]pyrazin-1-yl]-propionamide;
2-[[2-[(4-methoxy-2,6-dimethyl-phenyl)sulfonyl]-1,2,3,4-tetrahydro-pyrrolo[1,2-a]pyrazin-1-yl]-methoxy]-N-methyl-N-[cis-3-morpholin-4-yl-cyclohexyl]-acetamide;
4-[2-[(4-methoxy-2,6-dimethyl-phenyl)sulfonyl]-1,2,3,4-tetrahydro-pyrrolo[1,2-a]pyrazin-1-yl]-N-methyl-N-[cis-3-morpholin-4-yl-cyclohexyl]-butyramide;
2-[[2-[(2,6-dichloro-3-methyl-phenyl)sulfonyl]-1,2,3,4-tetrahydro-pyrrolo[1,2-a]pyrazin-1-yl]-methoxy]-N-methyl-N-[cis-3-morpholin-4-yl-cyclohexyl]-acetamide;
2-[2-[(6-methoxy-naphthalen-2-yl)sulfonyl]-1,2,3,4-tetrahydro-pyrrolo[1,2-a]pyrazin-1-yl]-N-methyl-N-[cis-3-morpholin-4-yl-cyclohexyl]-acetamide;
N-methyl-N-[cis-3-morpholin-4-yl-cyclohexyl]-3-[2-[[3-(trifluoromethyl)phenyl]sulfonyl]-1,2,3,4-tetrahydro-pyrrolo[1,2-a]pyrazin-1-yl]-propionamide;
N-[cis-3-(4,4-difluoro-piperidin-1-yl)-cyclohexyl]-2-[[2-[(4-methoxy-2,6-dimethyl-phenyl)sulfonyl]-1,2,3,4-tetrahydro-pyrrolo[1,2-a]pyrazin-1-yl]-methoxy]-N-methyl-acetamide;
N-[cis-3-(4,4-difluoro-piperidin-1-yl)-cyclohexyl]-4-[2-[(4-methoxy-2,6-dimethyl-phenyl)sulfonyl]-1,2,3,4-tetrahydro-pyrrolo[1,2-a]pyrazin-1-yl]-N-methyl-butyramide;
2-[[2-[(2,6-dichloro-3-methyl-phenyl)sulfonyl]-1,2,3,4-tetrahydro-pyrrolo[1,2-a]pyrazin-1-yl]-methoxy]-N-[cis-3-(4,4-difluoro-piperidin-1-yl)-cyclohexyl]-N-methyl-acetamide;
N-[cis-3-(4,4-difluoro-piperidin-1-yl)-cyclohexyl]-2-[2-[(6-methoxy-naphthalen-2-yl)sulfonyl]-1,2,3,4-tetrahydro-pyrrolo[1,2-a]pyrazin-1-yl]-N-methyl-acetamide;
N-[cis-3-(4,4-difluoro-piperidin-1-yl)-cyclohexyl]-N-methyl-3-[2-[[3-(trifluoromethyl)phenyl]sulfonyl]-1,2,3,4-tetrahydro-pyrrolo[1,2-a]pyrazin-1-yl]-propionamide;
2-[[2-[(4-methoxy-2,6-dimethyl-phenyl)sulfonyl]-1,2,3,4-tetrahydro-pyrrolo[1,2-a]pyrazin-1-yl]-methoxy]-N-methyl-N-[cis-3-(1,2,3,4-tetrahydro-[2,6]naphthyridin-2-yl)-cyclohexyl]-acetamide;
4-[2-[(4-methoxy-2,6-dimethyl-phenyl)sulfonyl]-1,2,3,4-tetrahydro-pyrrolo[1,2-a]pyrazin-1-yl]-N-methyl-N-[cis-3-(1,2,3,4-tetrahydro-[2,6]naphthyridin-2-yl)-cyclohexyl]-butyramide;
2-[[2-[(2,6-dichloro-3-methyl-phenyl)sulfonyl]-1,2,3,4-tetrahydro-pyrrolo[1,2-a]pyrazin-1-yl]-methoxy]-N-methyl-N-[cis-3-(1,2,3,4-tetrahydro-[2,6]naphthyridin-2-yl)-cyclohexyl]-acetamide;
2-[2-[(6-methoxy-naphthalen-2-yl)sulfonyl]-1,2,3,4-tetrahydro-pyrrolo[1,2-a]pyrazin-1-yl]-N-methyl-N-[cis-3-(1,2,3,4-tetrahydro-[2,6]naphthyridin-2-yl)-cyclohexyl]-acetamide; and
N-methyl-N-[cis-3-(1,2,3,4-tetrahydro-[2,6]naphthyridin-2-yl)-cyclohexyl]-3-[2-[[3-(trifluoromethyl)phenyl]sulfonyl]-1,2,3,4-tetrahydro-pyrrolo[1,2-a]pyrazin-1-yl]-propionamide;
or a physiologically compatible salt thereof.

18. A compound according to claim 17, wherein said compound is a hydrochloride salt.

19. A pharmaceutical composition comprising a compound according to claim 1 and at least one pharmaceutically acceptable excipient.

20. A method of treating a condition selected from the group consisting of pain; migraine; diabetes; diseases of the respiratory tract; inflammatory bowel diseases; neurological diseases; inflammations of the skin; rheumatic diseases; septic shock; reperfusion syndrome, and obesity, or of inhibiting angiogenesis, in a subject in need thereof, said method comprising administering to said subject a pharmaceutically effective amount of a compound according to claim 1.

21. A method according to claim 20, wherein said condition is pain selected from the group consisting of acute pain, visceral pain, neuropathic pain, chronic pain, and inflammatory pain.
